(19) United States
(12) Patent Application Publication
(10) Pub. No.: US 2005/0277630 A1
(43) Pub. Date: Dec. 15, 2005

(54) Antibacterial agents

(76) Inventors: Louis S. Chupak, Old Saybrook, CT (US); Wenhua Jiao, Groton, CT (US); Vera Prasad Venkata Nagendra Josyula, Ann Arbor, MI (US); Takushi Kaneko, Guilfor, CT (US)

Correspondence Address: Heidi M. Berven;Warner-Lambert Company LLC, 2800 Plymouth Road, Ann Arbor, MI, 48105, US

(21) Appl. No.: 10/772,860

(22) Filed: Feb. 6, 2004

Related U.S. Application Data
(60) Provisional application No. 60/445,909, filed on Feb. 7, 2003.

(51) Int. Cl. A61K 31/551; C07D 491/14

(52) U.S. Cl. 514/220; 540/560

(57) ABSTRACT

Compounds of formula I and methods for their preparation are disclosed. Further disclosed are methods of making biologically active compounds of formula I as well as pharmaceutically acceptable compositions comprising compounds of formula I. Compounds of formula I as disclosed herein can be used in a variety of applications including use as antibacterial agents.

This Regular Application claims benefit of U.S. Provisional Application No. 60/445,909, filed on Feb. 7, 2003.

### FIELD OF THE INVENTION

The invention relates to compounds bearing an oxazolidinone core structure which exhibit antibacterial activity, methods for their preparation, as well as pharmaceutically acceptable compositions comprising such compounds.

### BACKGROUND OF THE INVENTION

The oxazolidinones form a novel class of antibacterial agents with potent activity against a number of human and veterinary pathogens, including gram-positive aerobic bacteria such as multiply-resistant staphylococci and streptococci, anaerobic organisms such as bacteroides and clostridia species, and acid-fast organisms such as Mycobacterium tuberculosis and Mycobacterium. However, oxazolidinones generally do not demonstrate useful activity levels against aerobic gram-negative organisms. As a result, the use of oxazolidinones is limited to infections due to gram-positive bacteria. Accordingly, there is a need for oxazolidinones that have broader antibacterial activity, including activity against gram-negative as well as gram positive organisms.

### SUMMARY OF THE INVENTION

These and other needs are met by the present invention, which is directed to a compound of formula I:

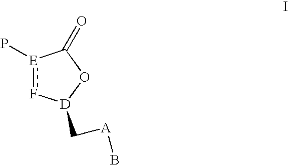

or a pharmaceutically acceptable salt thereof wherein:
A is O,
   NH, or
   S;
B is
   C(═O)R₁,
   C(═S)R₁,
   heterocylco,
   heteroaryl,
   C(═O)-heterocyclo, or
   C(═O)-heteteroaryl;
D is N, E is C, F is CH, and “—” is a bond, or D is CH, E is N, F is CH₂, and “—” is absent;
P is 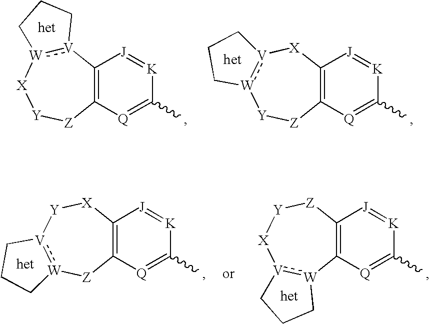
   wherein “ [symbol] ” indicates the point of attachment; and 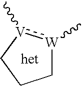
   is 5-membered heterocyclo or heteroaryl, wherein “ [symbol] ” indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;

V and W independently are CH or N when “ [symbol] ” is absent; or are C when “ [symbol] ” is a bond;

X, Y, Z independently are O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂,

provided that at least one of X, Y, or Z is NR₅,
N(C═O)R₅,
N(C═O)OR₅,
NSO₂R₅,
NSO₂NR₅,
O,
S,
SO, or
SO₂;

J, K, Q independently are CR₂ or N, with the proviso that when any one of J, K, or Q is N, then the other two are CR₂;

R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;

R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;

R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;

wherein n is 0, 1, 2, or 3;

R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;

wherein n is as defined above.

What is also provided is a compound of formula II 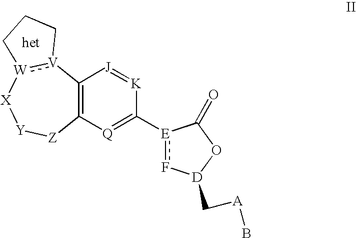
or a pharmaceutically acceptable salt thereof wherein:

A is O,
NH, or
S;

B is
C(═O)R₁,
C(═S)R₁,
heterocylco,
heteroaryl,
C(═O)-heterocyclo, or
C(═O)-heteteroaryl;

D is N, E is C, F is CH, and “—” is a bond, or D is CH, E is N, F is CH₂, and “—” is absent; 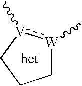
is 5-membered heterocyclo or heteroaryl, wherein “ [symbol] ” indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;

V and W independently are CH or N when “—” absent; or are C when “—” a bond;

X, Y, Z independently are O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂,

provided that at least one of X, Y, or Z is NR₅,
N(C═O)R₅,
N(C═O)OR₅,
NSO₂R₅,
NSO₂NR₅,
O,
S,
SO, or
SO₂;

J, K, Q independently are CR₂ or N, with the proviso that when any one of J, K, or Q is N, then the other two are CR₂;

R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;

R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;

R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;

wherein n is 0, 1, 2, or 3;

R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;

wherein n is as defined above.

What is also provided is a compound of formula III 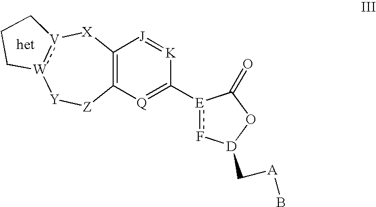
or a pharmaceutically acceptable salt thereof wherein:

A is O,
NH, or
S;

B is
C(═O)R₁,
C(═S)R₁,
heterocylco,
heteroaryl,
C(═O)-heterocyclo, or
C(═O)-heteteroaryl;

D is N, E is C, F is CH, and “—” is a bond, or D is CH, E is N, F is CH₂, and “—” is absent; 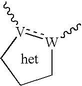
is 5-membered heterocyclo or heteroaryl, wherein “ [symbol] ” indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;

V and W independently are CH or N when “—” is absent; or are C when “—” is a bond;

X, Y, Z independently are O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂,

provided that at least one of X, Y, or Z is NR₅,
N(C═O)R₅,
N(C═O)OR₅,
NSO₂R₅,
NSO₂NR₅,
O,
S,
SO, or
SO₂;

J, K, Q independently are CR₂ or N, with the proviso that when any one of J, K, or Q is N, then the other two are CR₂;

R₁ is H,
(C₁-C₈)alkyl,
(C₃-C₆)cycloalkyl,
O—(C₁-C₄)alkyl,
O—(C₃-C₆)cycloalkyl,
S—(C₁-C₄)alkyl,
S—(C₃-C₆)cycloalkyl,
NH₂,
NH(C₁-C₄)alkyl,
N((C₁-C₄)alkyl)₂, or
NH—(C₃-C₆)cycloalkyl;

R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄)alkyl,
S—(C₃-C₆)cycloalkyl,
NH₂,
NH(C₁-C₄)alkyl,
N((C₁-C₄)alkyl)₂, or
NHC₃-C₆)cycloalkyl;

R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄)alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;

wherein n is 0, 1, 2, or 3;

R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;

wherein n is as defined above.

What is also provided is a compound of formula IV 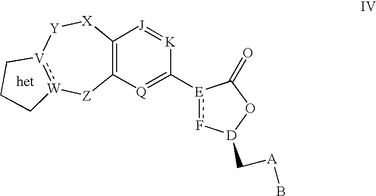
or a pharmaceutically acceptable salt thereof wherein:

A is O,
NH, or
S;

B is
C(═O)R₁,
C(═S)R₁,
heterocylco,
heteroaryl,
C(═O)-heterocyclo, or
C(═O)-heteteroaryl;

D is N, E is C, F is CH, and ”—” is a bond, or D is CH, E is N, F is CH₂, and “—” is absent; 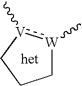
is 5-membered heterocyclo or heteroaryl, wherein “ [symbol] ” indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;

V and W independently are CH or N when “—” is absent; or are C when “—” is a bond;

X, Y, Z independently are O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂,

provided that at least one of X, Y, or Z is NR₅,
N(C═O)R₅,
N(C═O)OR₅,
NSO₂R₅,
NSO₂NR₅,
O,
S,
SO, or
SO₂;

J, K, Q independently are CR₂ or N, with the proviso that when any one of J, K, or Q is N, then the other two are CR₂;

R₁ is H,
(C₁-C₈)alkyl,
(C₃-C₆)cycloalkyl,
O—(C₁-C₄)alkyl,
O—(C₃-C₆)cycloalkyl,
S—(C₁-C₄)alkyl,
S—C₃-C₆)cycloalkyl,
NH₂,
NH(C₁-C₄)alkyl,
N((C₁-C₄)alkyl)₂, or
NH—(C₃-C₆)cycloalkyl;

R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;

R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄)alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;

wherein n is 0, 1, 2, or 3;

R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;

wherein n is as defined above.

What is also provided is a compound of formula V 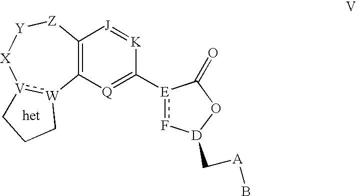
or a pharmaceutically acceptable salt thereof wherein:

A is O,
NH, or
S;

B is
C(═O)R₁,
C(═S)R₁,
heterocylco,
heteroaryl,
C(═O)-heterocyclo, or
C(═O)-heteteroaryl;

D is N, E is C, F is CH, and “—” is a bond, or D is CH, E is N, F is CH₂, and “—” is absent; 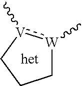
is 5-membered heterocyclo or heteroaryl, wherein “ [symbol] ” indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;

V and W independently are CH or N when “—” is absent; or are C when “—” is a bond;

X, Y, Z independently are O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂,

provided that at least one of X, Y, or Z is NR₅,
N(C═O)R₅,
N(C═O)OR₅,
NSO₂R₅,
NSO₂NR₅,
O,
S,
SO, or
SO₂;

J, K, Q independently are CR₂ or N, with the proviso that when any one of J, K, or Q is N, then the other two are CR₂;

R₁ is H,
(C₁-C₈)alkyl,
(C₃-C₆)cycloalkyl,
OC₁-C₄)alkyl,
O—(C₃-C₆)cycloalkyl,
SC₁-C₄)alkyl,
SC₃-C₆)cycloalkyl,
NH₂,
NH(C₁-C₄)alkyl,
N((C₁-C₄)alkyl)₂, or
NH—(C₃-C₆)cycloalkyl;

R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄)alkyl,
S—(C₃-C₆)cycloalkyl,
NH₂,
NH(C₁-C₄)alkyl,
N((C₁-C₄)alkyl)₂, or
NH—(C₃-C₆)cycloalkyl;

R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄)alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;

wherein n is 0, 1, 2, or 3;

R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;

wherein n is as defined above.

What is also provided is a compound which is

(S)—N-[3-(4,5-Dihydro-2H-6-oxa-1,2-diaza-benzo[e]azulen-8-yl)-2-oxo-oxazolidin-5-ylmethyl]-acetamide;

(S)—N-[3-(2-Methyl-4,5-dihydro-2H-6-oxa-1,2-diaza-benzo[e]azulen-8-yl)-2-oxo-oxazolidin-5-ylmethyl]-acetamide;

(S)—N-[3-(1-Methyl-4,5-dihydro-1H-6-oxa-1,2-diaza-benzo[e]azulen-8-yl)-2-oxo-oxazolidin-5-ylmethyl]-acetamide;

(S)—N-[3-(2-Ethyl-4,5-dihydro-2H-6-oxa-1,2-diaza-benzo[e]azulen-8-yl)-2-oxo-oxazolidin-5-ylmethyl]-acetamide;

(S)—N-[3-(1-Ethyl-4,5-dihydro-1H-6-oxa-1,2-diaza-benzo[e]azulen-8-yl)-2-oxo-oxazolidin-5-ylmethyl]-acetamide;

(S)—N-[3-(2-Benzyl-4,5-dihydro-2H-6-oxa-1,2-diaza-benzo[e]azulen-8-yl)-2-oxo-oxazolidin-5-ylmethyl]-acetamide;

(S)—N-[3-(1-Benzyl-4,5-dihydro-1H-6-oxa-1,2-diaza-benzo[e]azulen-8-yl)-2-oxo-oxazolidin-5-ylmethyl]-acetamide;

(S)—N-[2-Oxo-3-(2-phenethyl-4,5-dihydro-2H-6-oxa-1,2-diaza-benzo[e]azulen-8-yl)-oxazolidin-5-ylmethyl]-acetamide;

(S)—N-[2-Oxo-3-(1-phenethyl-4,5-dihydro-1H-6-oxa-1,2-diaza-benzo[e]azulen-8-yl)-oxazolidin-5-ylmethyl]-acetamide;

(S)—N-[2-Oxo-3-(3-phenyl-4,5-dihydro-2H-6-oxa-1,2-diaza-benzo[e]azulen-8-yl)-oxazolidin-5-ylmethyl]-acetamide;

(S)—N-[3-(2,6-Dihydro-4H-5-oxa-1,2-diaza-benzo[e]azulen-8-yl)-2-oxo-oxazolidin-5-ylmethyl]-acetamide;

(S)—N-[3-(5,6-Dihydro-2H-4-oxa-1,2-diaza-benzo[e]azulen-8-yl)-2-oxo-oxazolidin-5-ylmethyl]-acetamide;

(S)—N-[2-Oxo-3-(2,4,5,6-tetrahydro-1,2,6-triaza-benzo[e]azulen-8-yl)-oxazolidin-5-ylmethyl]-acetamide;

(S)—N-[2-Oxo-3-(2,4,5,6-tetrahydro-1,2,5-triaza-benzo[e]azulen-8-yl)-oxazolidin-5-ylmethyl]-acetamide;

(S)—N-[2-Oxo-3-(2,4,5,6-tetrahydro-1,2,4-triaza-benzo[e]azulen-8-yl)-oxazolidin-5-ylmethyl]-acetamide;

(S)—N-[3-(4,5-Dihydro-2H-6-thia-1,2-diaza-benzo[e]azulen-8-yl)-2-oxo-oxazolidin-5-ylmethyl]-acetamide;

(S)—N-[3-(2,6-Dihydro-4H-5-thia-1,2-diaza-benzo[e]azulen-8-yl)-2-oxo-oxazolidin-5-ylmethyl]-acetamide;

(S)—N-[3-(5,6-Dihydro-2H-4-thia-1,2-diaza-benzo[e]azulen-8-yl)-2-oxo-oxazolidin-5-ylmethyl]-acetamide;

(S)—N-[3-(6,6-Dioxo-2,4,5,6-tetrahydro-616-thia-1,2-diaza-benzo[e]azulen-8-yl)-2-oxo-oxazolidin-5-ylmethyl]-acetamide;

(S)—N-[3-(5,5-Dioxo-2,4,5,6-tetrahydro-516-thia-1,2-diaza-benzo[e]azulen-8-yl)-2-oxo-oxazolidin-5-ylmethyl]-acetamide;

(S)—N-[3-(4,4-Dioxo-2,4,5,6-tetrahydro-416-thia-1,2-diaza-benzo[e]azulen-8-yl)-2-oxo-oxazolidin-5-ylmethyl]-acetamide;

(S)—N-[3-(4,5-Dihydro-1,6-dioxa-2-aza-benzo[e]azulen-8-yl)-2-oxo-oxazolidin-5-ylmethyl]-acetamide;

(S)—N-[3-(4H,6H-1,5-Dioxa-2-aza-benzo[e]azulen-8-yl)-2-oxo-oxazolidin-5-ylmethyl]-acetamide;

(S)—N-[3-(5,6-Dihydro-1,4-dioxa-2-aza-benzo[e]azulen-8-yl)-2-oxo-oxazolidin-5-ylmethyl]-acetamide;

(S)—N-[3-(5,6-Dihydro-4H-1-oxa-2,6-diaza-benzo[e]azulen-8-yl)-2-oxo-oxazolidin-5-ylmethyl]-acetamide;

(S)—N-[3-(5,6-Dihydro-4H-1-oxa-2,5-diaza-benzo[e]azulen-8-yl)-2-oxo-oxazolidin-5-ylmethyl]-acetamide;

(S)—N-[3-(5,6-Dihydro-4H-1-oxa-2,4-diaza-benzo[e]azulen-8-yl)-2-oxo-oxazolidin-5-ylmethyl]-acetamide;

(S)—N-[3-(4,5-Dihydro-1-oxa-6-thia-2-aza-benzo[e]azulen-8-yl)-2-oxo-oxazolidin-5-ylmethyl]-acetamide;

(S)—N-[3-(4H,6H-1-Oxa-5-thia-2-aza-benzo[e]azulen-8-yl)-2-oxo-oxazolidin-5-ylmethyl]-acetamide;

(S)—N-[3-(5,6-Dihydro-1-oxa-4-thia-2-aza-benzo[e]azulen-8-yl)-2-oxo-oxazolidin-5-ylmethyl]-acetamide;

(S)—N-[3-(4,5-Dihydro-1-oxa-6-thia-2-aza-benzo[e]azulen-8-yl)-2-oxo-oxazolidin-5-ylmethyl]-acetamide;

(S)—N-[3-(5,5-Dioxo-5,6-dihydro-4H-1-oxa-516-thia-2-aza-benzo[e]azulen-8-yl)-2-oxo-oxazolidin-5-ylmethyl]-acetamide; or

(S)—N-[3-(5,6-Dihydro-1-oxa-4-thia-2-aza-benzo[e]azulen-8-yl)-2-oxo-oxazolidin-5-ylmethyl]-acetamide.

What is also provided is a pharmaceutical formulation comprising a compound of one of formulas I-V admixed with a pharmaceutically acceptable diluent, carrier, or excipient.

What is also provided is a method of treating a bacterial infection in a mammal, comprising administering to a mammal in need thereof an effective amount of a compound of one of formulas I-V.

### DETAILED DESCRIPTION OF THE INVENTION

Reference will now be made in detail to presently preferred compositions or embodiments and methods of the invention, which constitute the best modes of practicing the invention presently known to the inventors.

The term “alkyl” as used herein refers to a straight or branched hydrocarbon of from 1 to 11 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents selected from lower alkoxy, lower thioalkoxy, halogen, nitro, cyano, oxo, thio, —OH, —SH, —F, —CF₃, —OCF₃, —NO₂, —CO₂H, —CO₂C₁-C₆ alkyl, —NH₂, —NHC₁-C₆ alkyl, 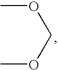
—CONR⁸R⁹, or —N(C₁-C₆alkyl)₂. Preferred alkyl groups have from 1 to 6 carbon atoms (C₁-C₆ alkyl).

The terms “(C₁-C₈)alkyl”, “(C₁-C₆)alkyl”, and “(C₁-C₄)alkyl” as used herein refer to subsets of alkyl which mean a straight or branched hydrocarbon radical having from 1 to 8, 1 to 6, or 1 to 4 carbon atoms respectively, and include, for example, methyl, ethyl, n-propyl, isopropyl, n-butyl, sec-butyl, isobutyl, tert-butyl, n-pentyl, n-hexyl, n-heptyl, and n-octyl and the like.

The term “(C₃-C₆)cycloalkyl” means a hydrocarbon ring containing from 3 to 6 carbon atoms, for example, cyclopropyl, cyclobutyl, cyclopentyl, or cyclohexyl. Where possible, the cycloalkyl group may contain double bonds, for example, 3-cyclohexen-1-yl. The cycloalkyl ring may be unsubstituted or substituted by one or more substituents selected from the following list, or as otherwise specifically indicated: alkyl, alkoxy, thioalkoxy, hydroxy, thiol, nitro, halogen, amino, alkyl and dialkylamino, formyl, carboxyl, CN, —NH—CO—R, —CO—NHR, —CO₂R, —COR, wherein R is defined as above, aryl, heteroaryl, wherein alkyl, aryl, and heteroaryl are as defined herein, or as indicated above for alkyl, alkenyl, and alkynyl substitutents. Examples of substituted cycloalkyl groups include fluorocyclopropyl, 2-iodocyclobutyl, 2,3-dimethylcyclopentyl, 2,2-dimethoxycyclohexyl, and 3-phenylcyclopentyl.

The term “halo” includes chlorine, fluorine, bromine, and iodine.

The term “aryl” means a cyclic or polycyclic aromatic ring having from 5 to 12 carbon atoms, and being unsubstituted or substituted with one or more of the substituent groups recited above for alkyl groups including, halogen, nitro, cyano —OH, —SH, —F, —CF₃, —OCF₃, —N₂, 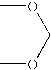
—CO₂H, —CO₂C₁-C₆ alkyl, —NH₂, —NHC₁-C₆ alkyl, —CONR^{a}R^{b}, wherein R^{a} and R^{b} are H or (C₁-C₆)alkyl or (C₃-C₆)cycloalkyl, SO₂alkyl, —SO₂NH₂, or —N(C₁-C₆alkyl)₂. Examples include, but are not limited to phenyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, thienyl, naphthyl, 4-thionaphthyl, tetralinyl, anthracinyl, phenanthrenyl, benzonaphthenyl, fluorenyl, 2-acetamidofluoren-9-yl, and 4′-bromobiphenyl.

The term “heteroaryl” means an aromatic cyclic or polycyclic ring system having from 1 to 4 heteroatoms selected from N, O, and S. Typical heteroaryl groups include 2- or 3-thienyl, 2- or 3-furanyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridinyl, 3-, 4-, or 5-pyridazinyl, 2-pyrazinyl, 2-, 4-, or 5-pyrimidinyl, 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. The heteroaryl groups may be unsubstituted or substituted by 1 to 3 substituents selected from those described above for alkyl, alkenyl, and alkynyl, for example, cyanothienyl and formylpyrrolyl. Preferred aromatic fused heterocyclic rings of from 8 to 10 atoms include but are not limited to 2-, 3-, 4-, 5-, 6-, 7-, or 8-quinolinyl, 1-, 3-, 4-, 5-, 6-, 7-, or 8-isoquinolinyl-, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. Heteroaryl also includes 2- and 3-aminomethylfuran, 2- and 3-aminomethylthiophene and the like.

The term “heterocyclic” means monocyclic, fused, bridged, or spiro bicyclic heterocyclic ring systems. Monocyclic heterocyclic rings contain from about 3 to 12 ring atoms, with from 1 to 5 heteroatoms selected from N, O, and S, and preferably from 3 to 7 member atoms, in the ring. Bicyclic heterocyclics contain from about 5 to about 17 ring atoms, preferably from 5 to 12 ring atoms. Bicyclic heterocyclic rings may be fused, spiro, or bridged ring systems. Examples of heterocyclic groups include cyclic ethers (oxiranes) such as ethyleneoxide, tetrahydrofuran, dioxane, and substituted cyclic ethers, wherein the substituents are those described above for the alkyl and cycloalkyl groups. Typical substituted cyclic ethers include propyleneoxide, phenyloxirane (styrene oxide), cis-2-butene-oxide (2,3-dimethyloxirane), 3-chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiophen-4-yl and substituted groups such as aminomethyl thiophene. Other commonly employed heterocycles include dihydro-oxathiol-4-yl, dihydro-1H-isoindole, tetrahydro-oxazolyl, tetrahydro-oxadiazolyl, tetrahydrodioxazolyl, tetrahydrooxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene.

In the context of the present invention, the terms “5-membered heterocyclo” and “5-membered heteroaryl” refer to 5-membered heterocyclo- and heteroaryl groups that fall within the scope of the definitions provided above, or more particularly are summarized in Table 1.

TABLE 1

| | | | | |
|---|---|---|---|---|
| 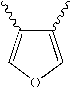 | 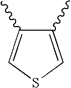 | 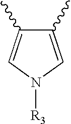 | 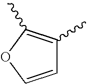 | 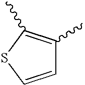 |

| | | | | |
|---|---|---|---|---|
| 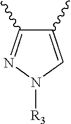 | 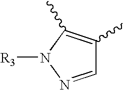 | 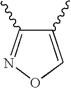 | 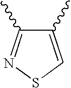 | 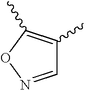 |

| | | | |
|---|---|---|---|
| 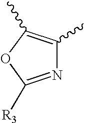 | 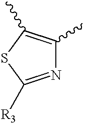 | 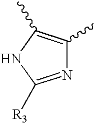 | 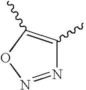 |
| 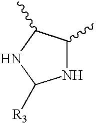 | 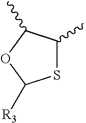 | 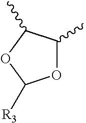 | 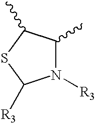 |

| | | |
|---|---|---|
| 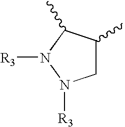 | 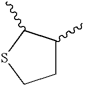 | 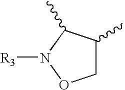 |
| 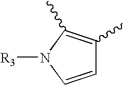 | 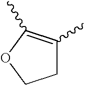 | 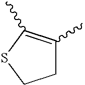 |
| 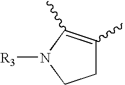 | 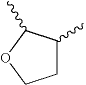 | 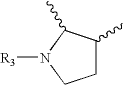 |

| | | | |
|---|---|---|---|
| 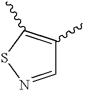 | 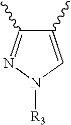 | 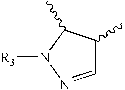 | 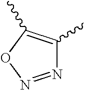 |
| 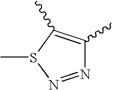 | 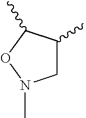 | 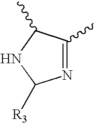 | 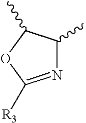 |
| 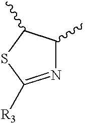 | 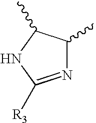 | 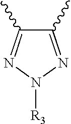 | |

A bond represented by a line such as “—” is meant to represent that the bond may be absent or present, provided that the resultant compound is stable and of satisfactory valency.

The term “patient” means all mammals, including humans. Other examples of patients include cows, dogs, cats, goats, sheep, pigs, and rabbits.

A “therapeutically effective amount” is an amount of a compound of the present invention that, when administered to a patient, elicits the desired therapeutic effect; i.e., inhibits bacterial infection.

It will be appreciated by those skilled in the art that compounds of the invention having one or more chiral centers may exist in and be isolated in optically active and racemic forms. Some compounds may exhibit polymorphism. It is to be understood that the present invention encompasses any racemic, optically-active, polymorphic, geometric, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity or cytotoxicity using the standard tests described herein, or using other similar tests which are well known in the art.

A “prodrug” is an inactive derivative of a drug molecule that requires a chemical or an enzymatic biotransformation in order to release the active parent drug in the body.

Specific and preferred values for compounds of Formula I are listed below for radicals, substituents, and ranges are for illustration purposes only, and they do not exclude other defined values or other values within defined ranges for the radicals and substituents.

Thus, turning now to formula I, a specific value for 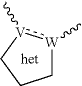
is any value disclosed in Table 1.

A specific value for A is NH, as designated in formula IA.

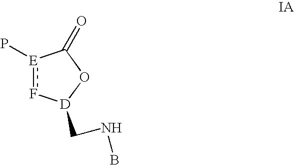

A specific value for B is acetyl as designated in formula IB.

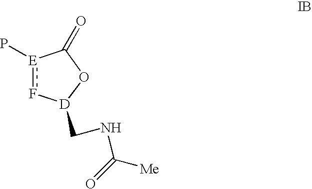

Specific values for D, E, and F, are CH, N, and CH₂, respectively, as designated in formula IC.

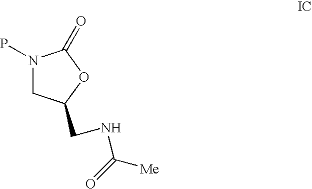

A secific value for P is 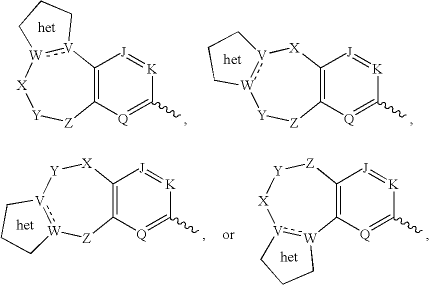
wherein J, K, and Q and V, W, X, Y, and Z have any of the manings described herein.

A more specific value for P is 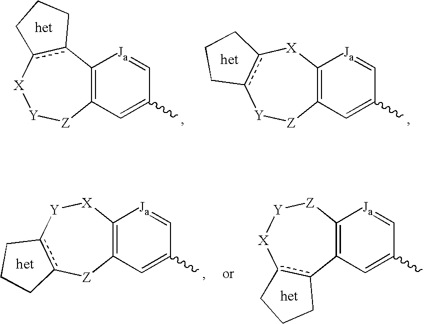
wherein Jₐ is N or CR₁₀, wherein R₁₀ is H or F, “ [symbol] ” indicates the point of attachment, and wherein

one or two of X, Y, or Z is NR₅, N(C═O)R₅, N(C═O)OR₅, NSO₂R₅, NSO₂NR₅, O, S, SO, or SO₂.

Turning now to a compound of formula II, a specific value for 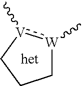
is as defined for 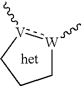
in compounds of formula I.

A specific value for A is NH, as designated in formula IIA.

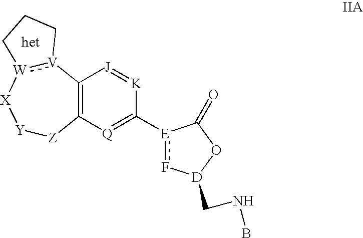

A specific value for B is acetyl, as designated in formula IIB.

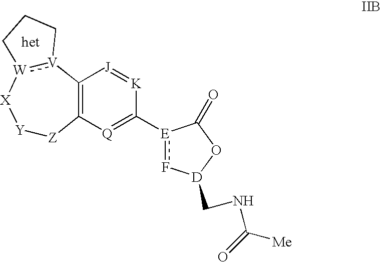

Specific values for D, E, and F, are CH, N, and CH₂, respectively, as designated in formula IIC.

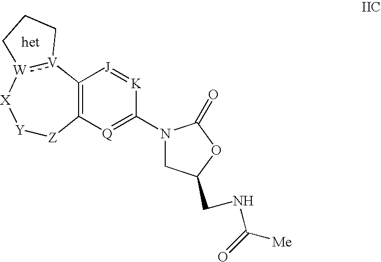

As designated in formula IID, a specific value for J is Ja, wherein Jₐ is N or CR₁₀, wherein R₁₀ is H or F. Specific values for K and Q are CH, and CH, respectively

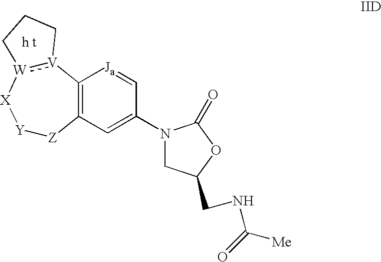

Specific values for X, Y, and Z, are as designated in formula IIE, wherein only one or two of X, Y, or Z is NR₅, N(C═O)R₅, N(C═O)OR₅, NSO₂R₅, NSO₂NR₅, O, S, SO, or SO₂.

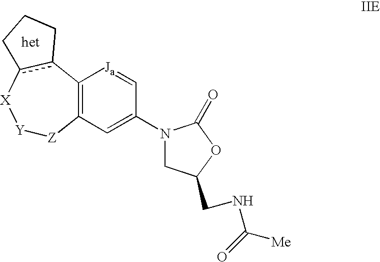

Specific values for Y and Z are as designated in formula IIF, wherein Xₐ is NR₅, N(C═O)R₅, N(C═O)OR₅, NSO₂R₅, NSO₂NR₅, O, S, SO, or SO₂.

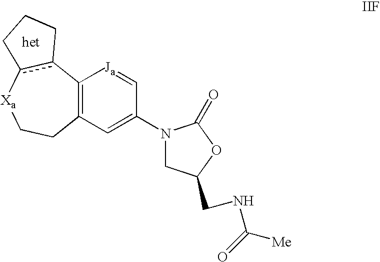

Specific values for X and Z are as designated in formula IIG, wherein Yₐ is NR₅, N(C═O)R₅, N(C═O)OR₅, NSO₂R₅, NSO₂NR₅, O, S, SO, or SO₂.

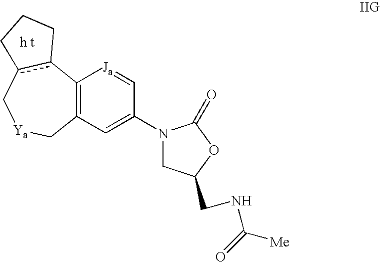

Specific values for X and Y are as designated in formula IIH, wherein Zₐ is NR₅, N(C═O)R₅, N(C═O)OR₅, NSO₂R₅, NSO₂NR₅, O, S, SO, or SO₂.

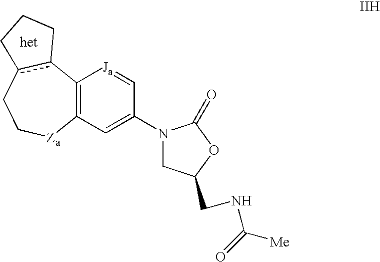

Turning now to compounds of formula III, a specific value for 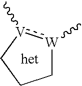
is as defined for 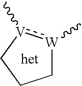
in formula I.

A specific value for A is NH as designated in formula IIIA.

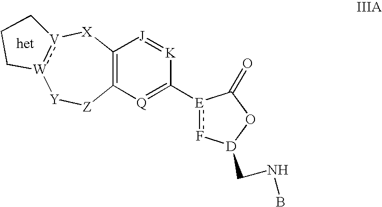

A specific value for B is acetyl as designated in formula IIIB.

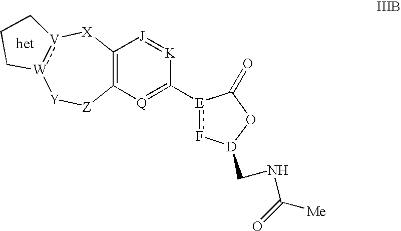

Specific values for D, E, and F, are CH, N, and CH₂, respectively, as designated in formula IIIC.

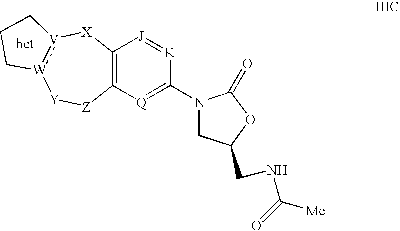

As designated in formula IIID, a specific value for J is Ja, wherein Ja is N or CR₁₀, wherein R₁₀ is H or F. Specific values for K and Q are CH, and CH, respectively.

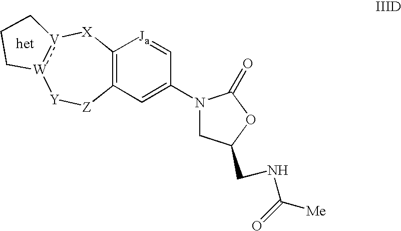

Specific values for X, Y, and Z, are as designated in formula IIE, wherein only one or two of X, Y, or Z is NR₅, N(C═O)R₅, N(C═O)OR₅, NSO₂R₅, NSO₂NR₅, O, S, SO, or SO₂.

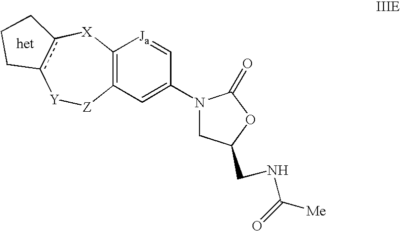

Specific values for Y and Z are as designated in formula IIIF, wherein Xₐ is NR₅, N(C═O)R₅, N(C═O)OR₅, NSO₂R₅, NSO₂NR₅, O, S, SO, or SO₂.

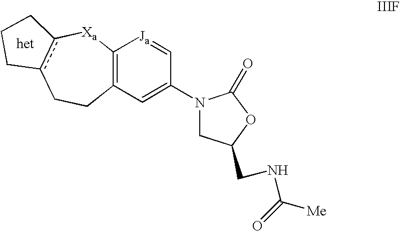

Specific values for X and Z are as designated in formula IIIG, wherein Yₐ is NR₅, N(C═O)R₅, N(C═O)OR₅, NSO₂R₅, NSO₂NR₅, O, S, SO, or SO₂.

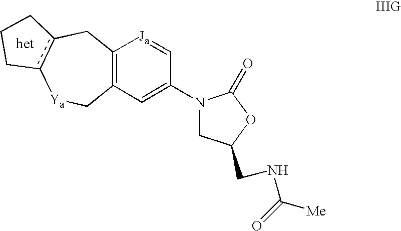

Specific values for X and Y are as designated in formula IIIH, wherein Zₐ is NR₅, N(C═O)R₅, N(C═O)OR₅, NSO₂R₅, NSO₂NR₅, O, S, SO, or SO₂.

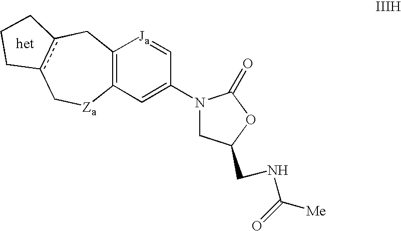

Turning now to a compound of formula IV, a specific value for 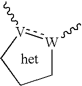
is as defined for 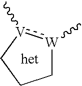
in formula I.

A specific value for A is NH as designated in formula IVA.

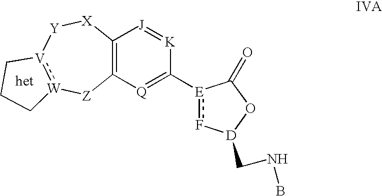

A specific value for B is acetyl as designated in formula IVB.

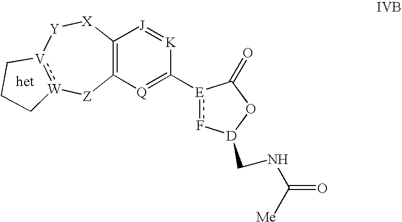

Specific values for D, E, and F, are CH, N, and CH₂, respectively, as designated in formula IVC.

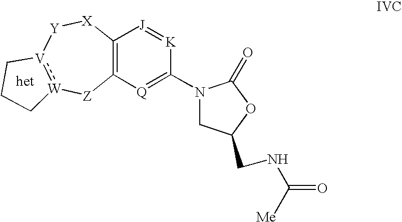

As designated in formula IVD, a specific value for J is Ja, wherein Jₐ is N or CR₁₀, wherein R₁₀ is H or F. Specific values for K and Q are CH, and CH, respectively.

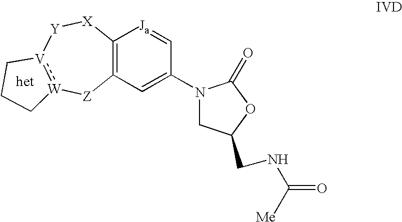

Specific values for X, Y, and Z, are as designated in formula IVE, wherein only one or two of X, Y, or Z is NR₅, N(C═O)R₅, N(C═O)OR₅, NSO₂R₅, NSO₂NR₅, O, S, SO, or SO₂.

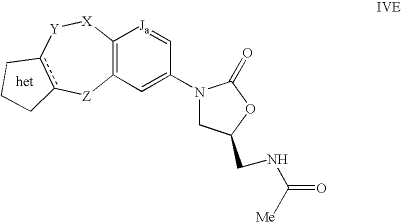

wherein R₈ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl, wherein n is 0, 1, 2, or 3; or taken together R₈ and R₉ are bonded to the same C and form C═O.

Specific values for Y and Z are as designated in formula IVF, wherein Xₐ is NR₅, N(C═O)R₅, N(C═O)OR₅, NSO₂R₅, NSO₂NR₅, O, S, SO, or SO₂.

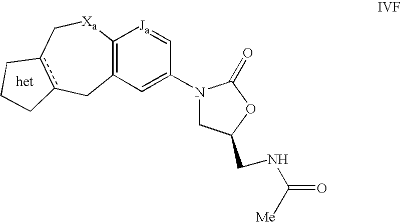

Specific values for X and Z are as designated in formula IVG, wherein Yₐ is NR₅, N(C═O)R₅, N(C═O)OR₅, NSO₂R₅, NSO₂NR₅, O, S, SO, or SO₂.

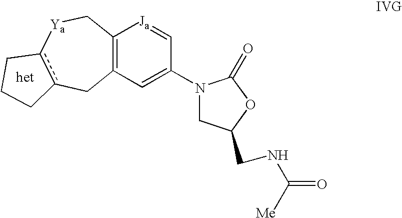

Specific values for X and Y are as designated in formula IVH, wherein Zₐ is NR₅, N(C═O)R₅, N(C═O)OR₅, NSO₂R₅, NSO₂NR₅, O, S, SO, or SO₂.

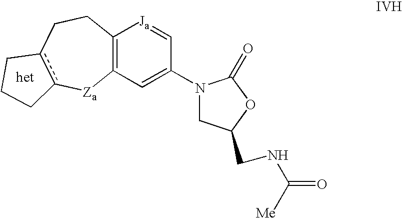

Turning now to a compound of formula V, a specific value 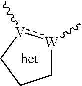
for is as defined for 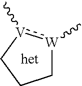
in formula I.

A specific value for A is NH as designated in formula VA.

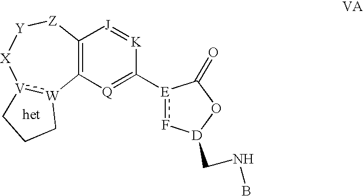

A specific value for B is acetyl as designated in formula VB.

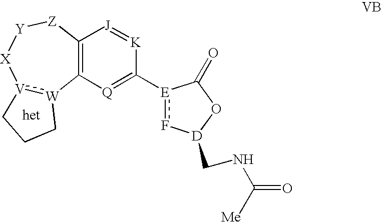

Specific values for D, E, and F, are CH, N, and CH₂, respectively, as designated in formula VC.

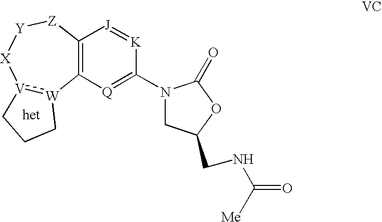

As designated in formula IVD, a specific value for J is Ja, wherein Jₐ is N or CR₁₀, wherein R₁₀ is H or F. Specific values for K and Q are CH, and CH, respectively.

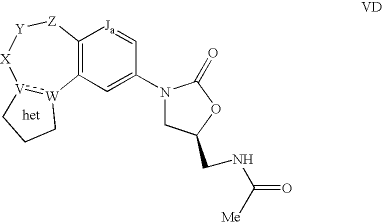

Specific values for X, Y, and Z, are as designated in formula IIE, wherein only one or two of X, Y, or Z is NR₅, N(C═O)R₅, N(C═O)OR₅, NSO₂R₅, NSO₂NR₅, O, S, SO, or SO₂.

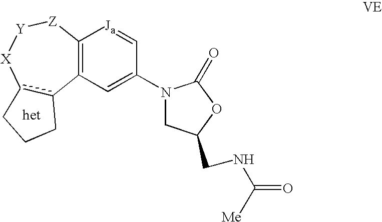

Specific values for Y and Z are as designated in formula VF, wherein Xₐ is NR₅, N(C═O)R₅, N(C═O)OR₅, NSO₂R₅, NSO₂NR₅, O, S, SO, or SO₂.

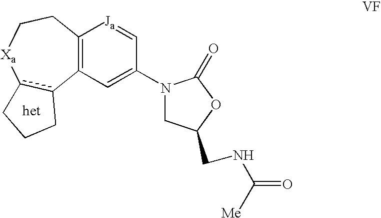

Specific values for X and Z are as designated in formula VG, wherein Yₐ is NR₅, N(C═O)R₅, N(C═O)OR₅, NSO₂R₅, NSO₂NR₅, O, S, SO, or SO₂.

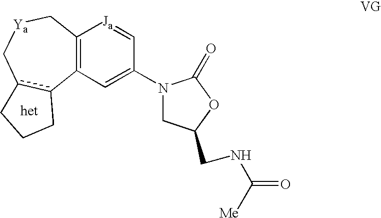

Specific values for X and Y are as designated in formula VH, wherein Zₐ is NR₅, N(C═O)R₅, N(C═O)OR₅, NSO₂R₅, NSO₂NR₅, O, S, SO, or SO₂.

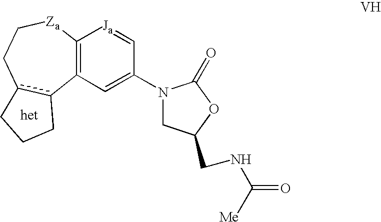

### Preparation of Invention Compounds

As is readily apparent from this disclosure, compounds of the present invention are characterized by a fused tricyclic subunit, covalently attached to a oxazolidinyl subunit. As depicted retrosynthetically in Scheme I, the invention compounds can be prepared from the corresponding bicyclo oxazolidinone intermediate via annelation procedures known to the skilled artisan. One useful platform for elaborating the third ring of the tricyclic subunit recognizable to the skilled artisan is thus the corresponding bicyclic ketone (e.g., V, W, X, Y, or Z is C═O). Many other platforms are available, depending on functional groups present in the cycloheptyl portion of the bicyclo subunit. The bicyclo oxazolidinone intermediates are prepared via covalent attachment of the bicyclo subunit under alkylation (X is NHR, wherein R is a protecting group) or coupling (X is halo, triflate, or another group known to the skilled artisan, that is susceptible to coupling) conditions, to the oxazolidinone core. Methods for the preparation of the requisite bicyclo and oxazolidinone subunits are also readily available to the skilled artisan.

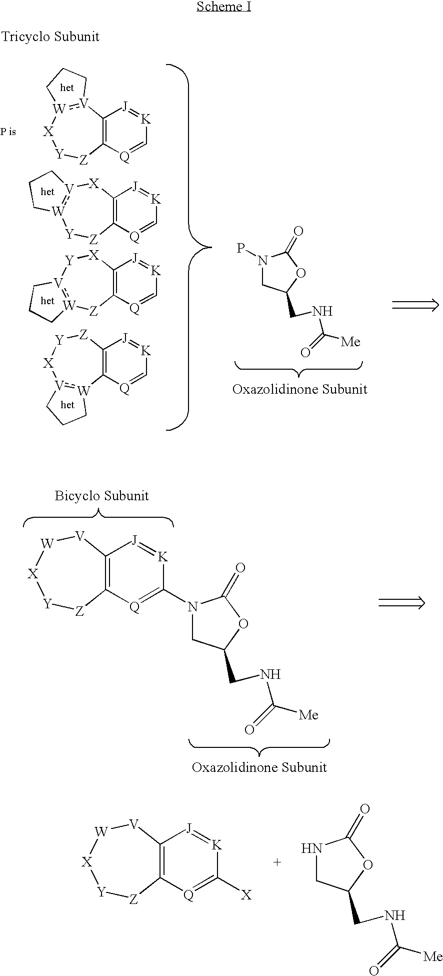

Alternatively, as depicted in Scheme II, the elaborated tricyclo subunit wherein X is halo, triflate, or another group known to the skilled artisan that is susceptible to coupling conditions, may be directly appended to the oxazolidinone core.

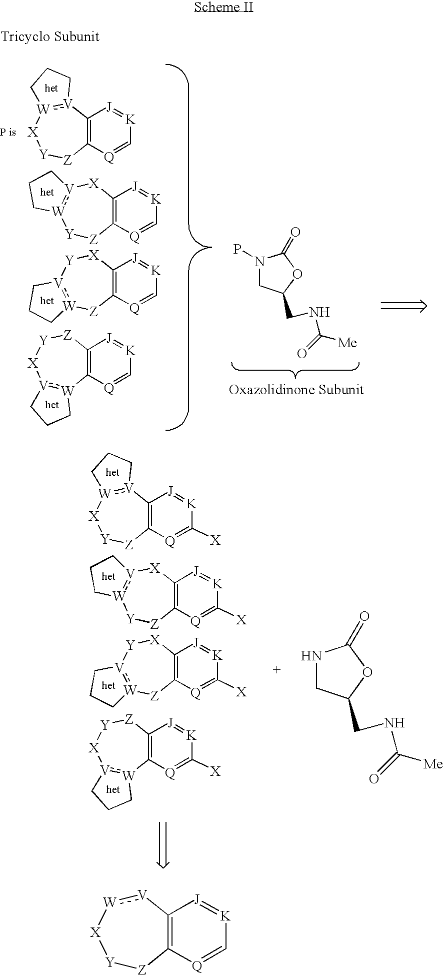

Again alternatively, as depicted in Scheme III, the oxazolidinyl subunit can be elaborated from the corresponding acetamides III-1 or III-2 via treatment with the epoxide or halo acetate, as shown.

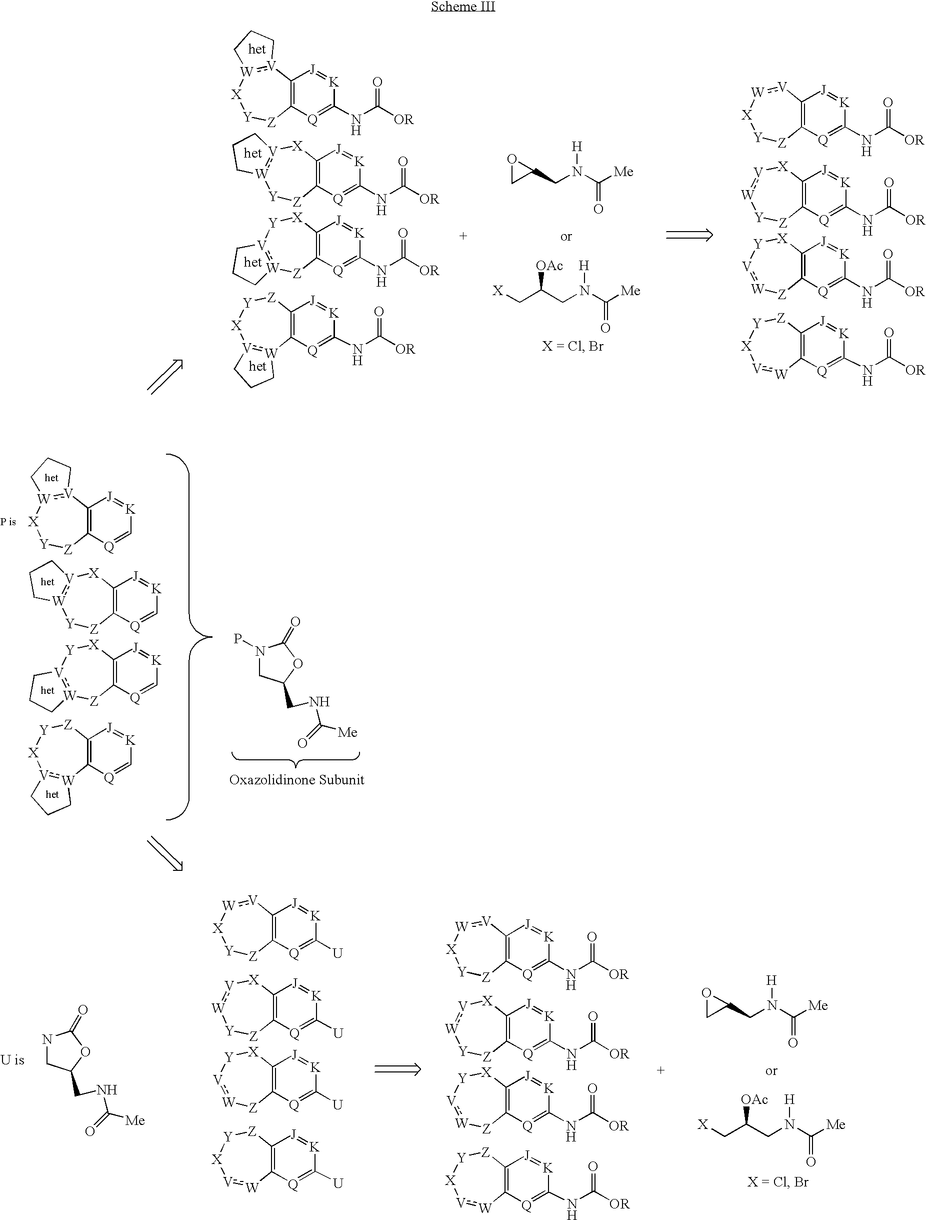

Reflecting the synthetic strategies summarized in Schemes I, II, and III, the following section describing the preparation of the invention compounds has three sections. The first section summarizes the preparation of common intermediates (for instance, the oxazolidinone core). The second section summarizes the preparation and attachment of bicyclo subunits to the oxazolidinyl core to provide the bicyclo oxazolidinone intermediates. The third section summarizes the elaboration of the tricyclo subunit using either the bicyclo subunit or bicyclo oxazolidinone intermediate as a platform.

1. Preparation of Common Intermediates

The following compounds which were used in the synthesis of the compounds of the invention were prepared as follows.

### (R)-5-Hydroxymethyl-oxazolidin-2-one

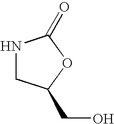

The title compound was prepared according to the procedure described by K. Danielmeier and E. Steckhan in Tetrahedron Assymetry 1995, 6(5), 1181-1190.

### N-(2,4-Dimethoxy-benzyl)-N-(2-oxo-oxazolidin-5-ylmethyl)-acetamide

The title compound was prepared as described in Tetrahedron Letters, 2001, 42, 3681.

### (S)—N-Oxiranylmethyl-acetamide

To a solution of (S)—N-acetyl-3-bromo-2-acetoxypropylamine (5 g, 0.021 mmol) in acetonitrile (20 mL) and methanol (20 mL) was added potassium carbonate (0.021 mmol) portion-wise. The reaction mixture was stirred at 0° C. for 1 hour and then warmed to room temperature slowly and stirred overnight. To it 50 mL of ethyl acetate was added and the precipitate was removed by filtration. Organic solvents were removed and the residue was dissolved in 60 mL of ethyl acetate and remaining precipitate was filtered and organic solution was concentration under reduced pressure to yield 1.6 g (90% yield) to obtain the title compound.

2. Preparation of Heterobicyclo-Containing Oxazolidinone Intermediates

Approaches to the preparation of the bicyclo-containing intermediates are depicted generally in the following schemes. Thus, in Scheme 1, nitration of bicyclo cycloheptanone 1A-1 (step I) provides nitro compound 1A-2, which is subsequently reduced to the amine 1A-3 (step II). Protection of the amine moiety in 1A-3 (step III), followed by treatment with (R)-gycidol butyrate provides oxazolidinone 1A-5 (step IV). Mesylation of the alcohol moiety in 1A-5 (step V), followed by treatment with sodium azide, provides azide 1A-7 (step VI). Hydrogenation (step VII) and acetylation (step VII) provides the target compound 1A-9.

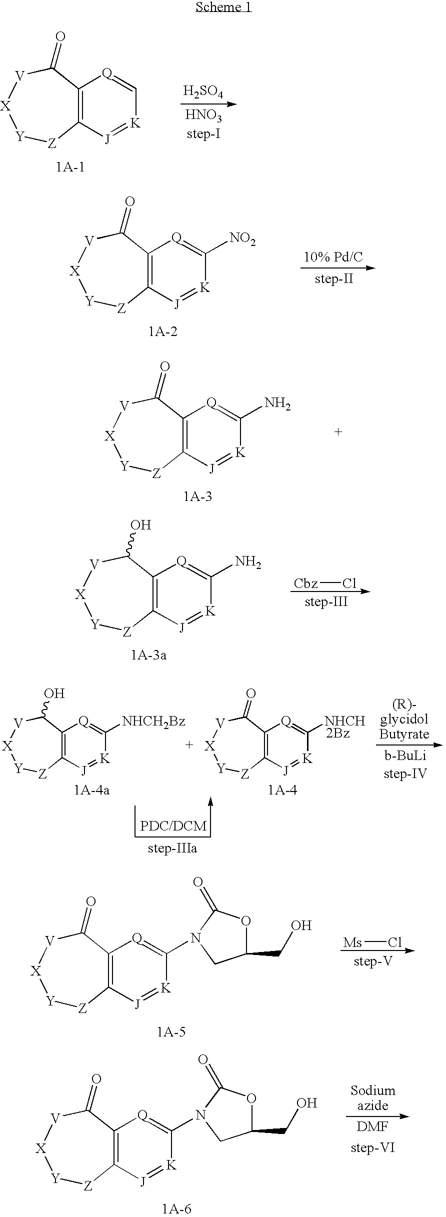

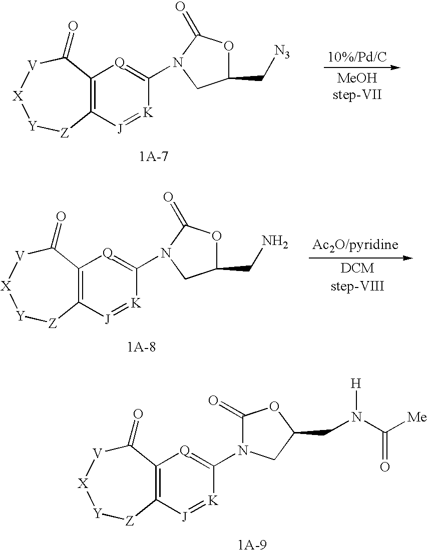

Scheme 1B provides a variant of the Scheme 1A approach wherein the keto moiety is “walked” around the ring. Nitration of ketone 1B-1 (step I) provides nitro compound 1B-2, which is reduced to the corresponding amine 1B-3 (step II) under conditions known to the skilled artisan. Protection of the amine moiety (step III), followed by attachment of the oxazolidinone core using reagents known to the skilled artisan provides 1B-5. Elaboration of the acetamide sidechain of the oxazolidinone subunit in 1B-5 commences with formation of the mesylate or an equivalent (step VI), followed by displacmement with azide, reduction (step VII) and acetylation (step VI) to provide the target compound 1B-9.

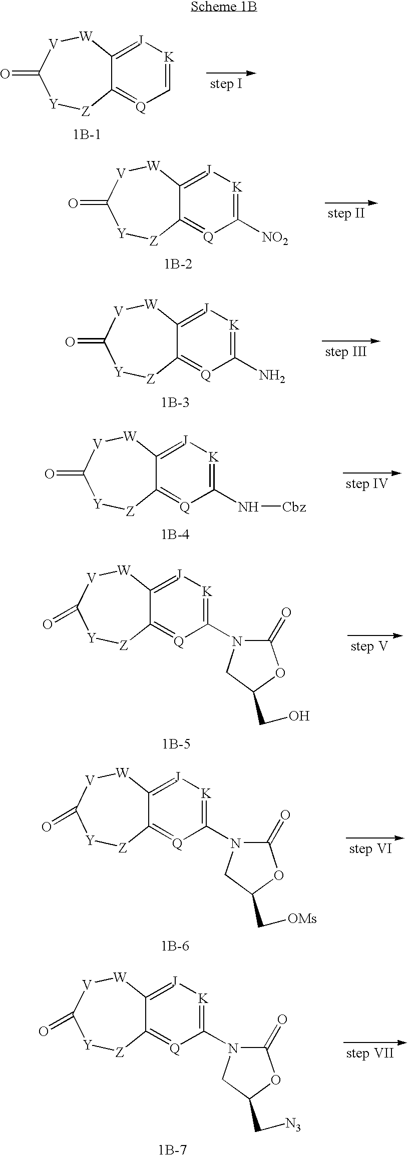

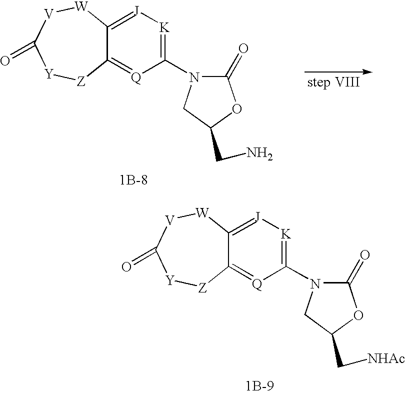

Scheme 1C provides another variant of the Scheme 1A approach wherein keto moiety is “walked” around the ring. Thus, the keto moiety in compound 1C 1 is converted to the exo methylene compound 1C-2 (step I). Epoxidation and ring enlargement of 1C-2 affords ketone 1C-3. Coupling of compound 1C-2 to the oxazolidinone subunit (step III) provides 1C4. Elaboration of the acetamide sidechain of the oxazolidinone subunit is as provided in Scheme 1B.

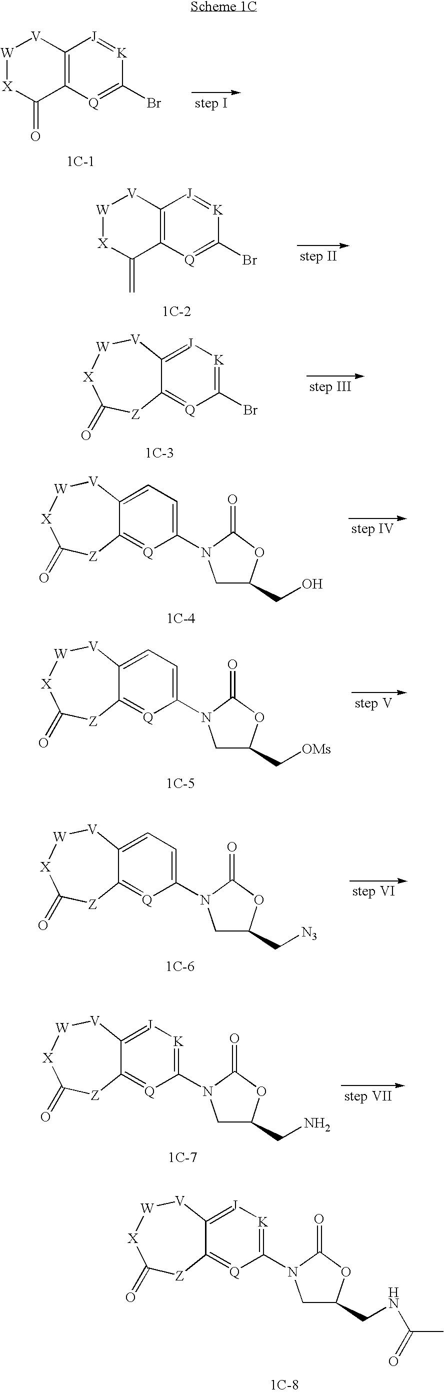

Scheme 1D provides a variant of the Scheme 1C approach. Thus, deprotection and bromination of 1D-1 (step I) provides compound 1D-2. Steps II and III are similar to steps II and III in Scheme 1C. Coupling (step IVB) and deprotection (step V) provide the target compound 1D-6.

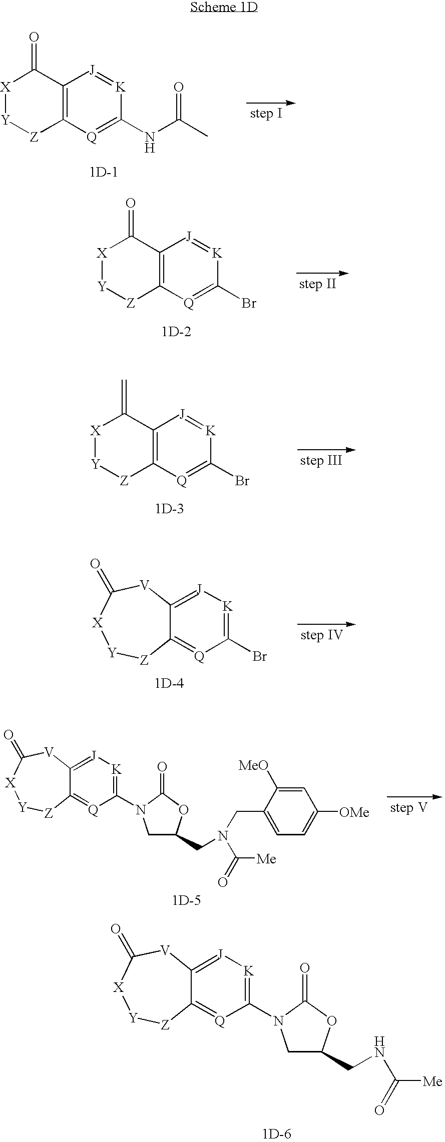

Scheme 2 provides alternative approaches to the attachment of the oxazolidinone subunit of the invention compounds to the fused bicyclo ketone subunit. The method commences with bromination of 2-1 to provide 2-2 (step I), followed by reduction of the ketone moiety (step II) to provide alocohol 2-3. The alcohol moiety in 2-3 is removed by techniques known to the skilled artisan (step III), for instance, via conversion to a leaving group such as a mesylate or tosylate, followed by reduction using a trialkyl tin hydride, to provide bromide 2-4. A variety of coupling procedures may be used to couple bromide 2-4 to the requisite N-protected acetamide 2-4a (step IV) to provide the protected core 2-5. Deprotection and oxidation provides the target compound.

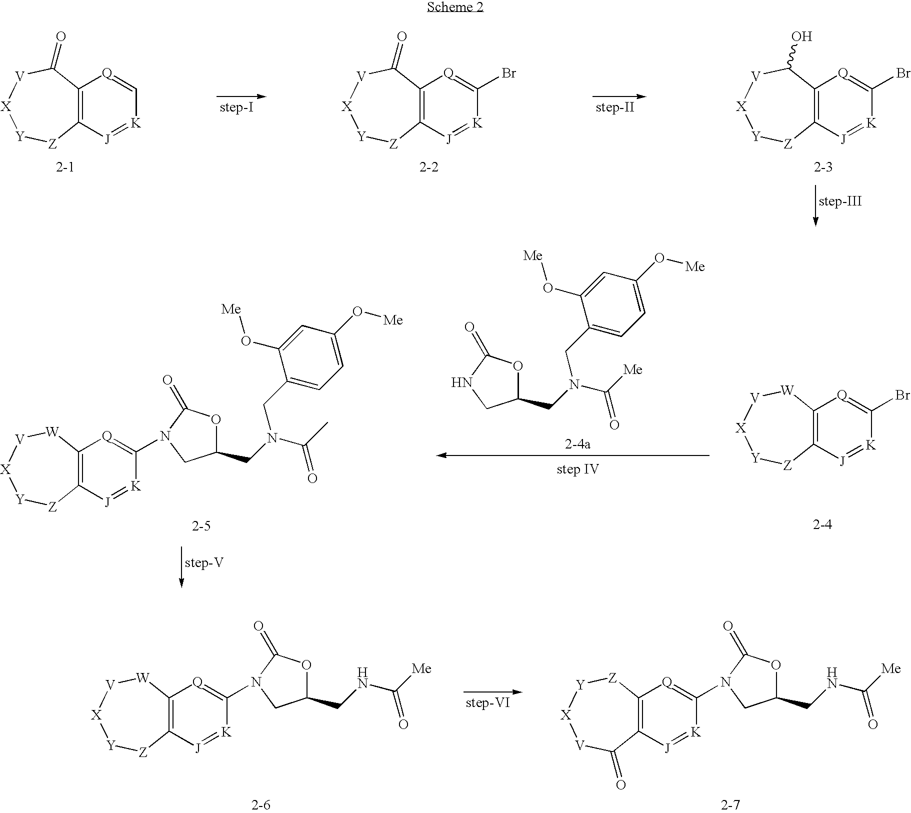

Scheme 3 provides an approach to ozepin, azepin, and thiepin-type systems. Thus, ethyl 4-bromo-butanoate is coupled with the aryl bromide 3-1, wherein X is OH, SH, NH₂, or NH (C₁-C₆)alkyl (step 1) to provide 4-2. Saponification of 4-2, followed by cyclization and elaboration of the oxazolidinone subunit provides the target compound e3-6.

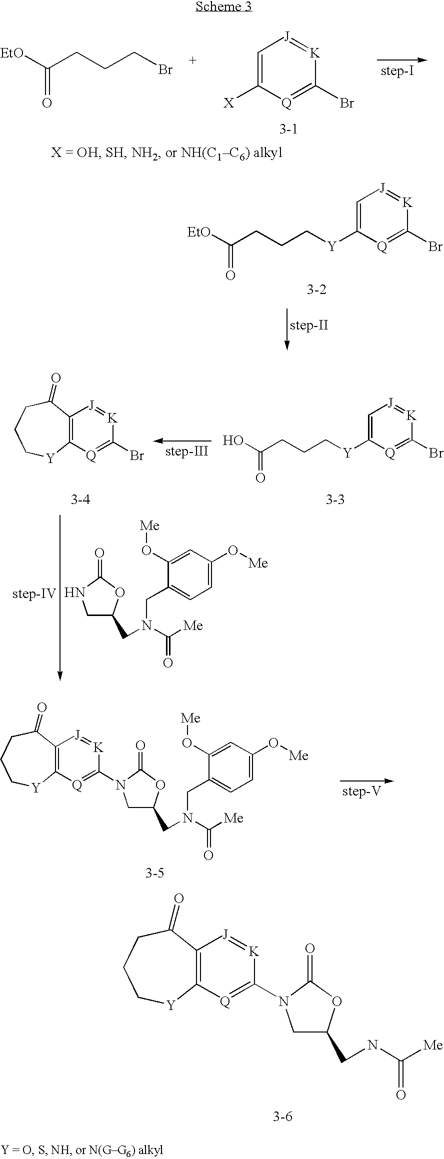

Scheme 4 provides a route for the preparation of sulfone-containing bicyclo oxazolidinone cores from the corresponding thioethers (e.g., compound 3-6 wherein Y is S). Thus, oxidation of the thioether moiety in 3-6 (step 1), followed by deprotection (step 2), provides sulfone 4-2. 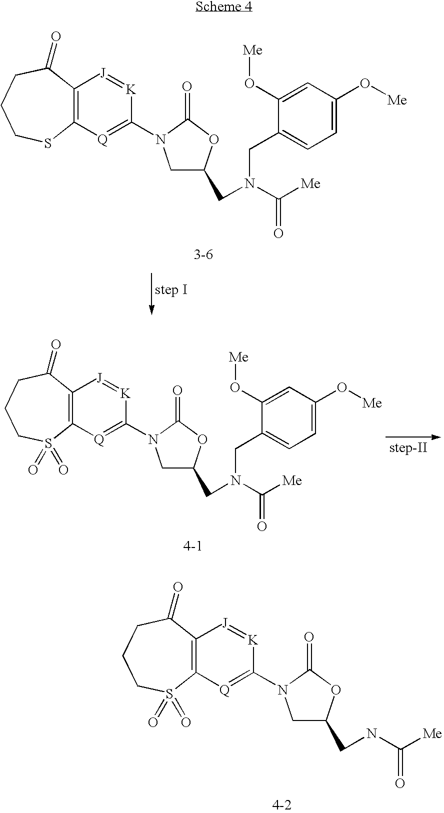
3. Preparation of Fused Bicyclo-Containing Oxazolidinones

The preparation of invention compounds from heterobicyclo intermediates is described below. Schemes 5A-J depict the preparation of an invention compound incorporating a fused diazinyl ring. Treatment of compound 1A-9 (Scheme 2B) with DMF acetal in Scheme 5A provides enamine 5A-1. Enamine 5A-1 can be treated with hydrazine or an alkyl substituted Hydrazine to provide diazines 5A-2 and 5A-3, which can be separated using conventional techniques such as silica gel chromatography.

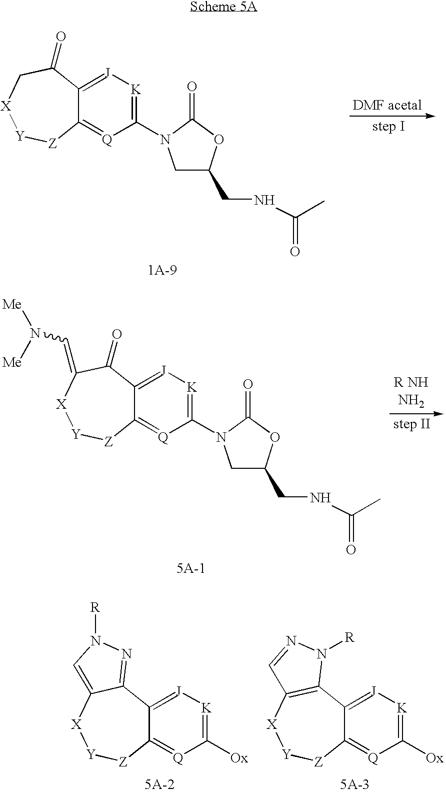

Scheme 5B provides an alternative strategy for the preparation of substituted fused diazines. Thus compound 1A-9 is treated with an acid chloride or anhydride to provide the β-diketo compound 5B-1 (step I). As in Scheme 5A, treatment of compound 5B-1 with hydrazine or an alkyl-substituted hydrazine (step II) provides diazines 5B-2 and 5B-3, which can be separated using conventional techniques such as silica gel chromatography. Alternatively, compound 1A-9 can be treated directly with hydrazine or an alkyl substituted hydrazine (step III) to provide the cycloheptylidene hydrazine derivative 5B4. Treatment of compound 5B-4 with base and an ester (step IV) provides the fused diazinyl target compound 5B-5.

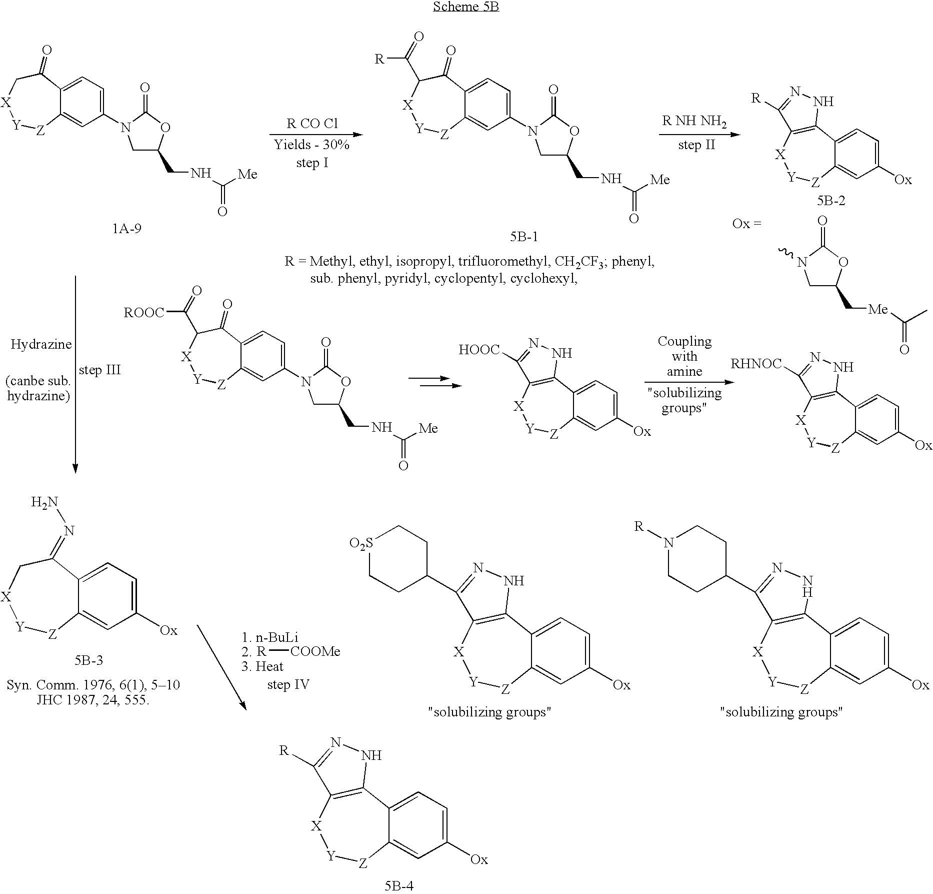

Scheme 5C provides an alternative approach to the synthesis of fused substituted diazinyl systems, that focuses on the preparation of invention compounds with enhanced solubilities. Thus compound 5C-1, which is readily prepared according to methods available to the skilled artisan, is converted to the diazinyl system 5C-2 (step I) as provided in Schemes 5A and 5B. The acid moiety in compound 5C-2 provides a platform for appending various solubilizing groups on the invention compound skeleton, such as depicted in compounds 5C-3, 5C4, and 5C-5.

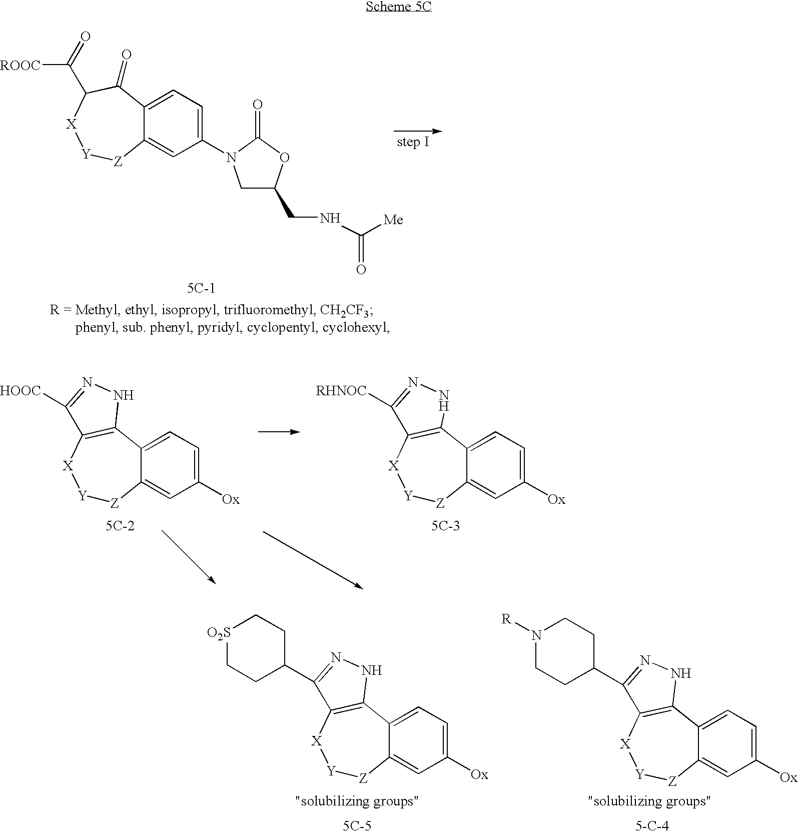

Scheme 5D summarizes an alternative strategy for the preparation of substituted diazinyl systemts. Thus, alkylation of 1A-9 using base and diethyloxalate, followed by treatment with hydrazine or substituted hydrazine provides the hydroxymethyl-substituted diazine 5D-1. Compound 5D-1 can be converted to the substituted amine 5D-2 via conversion of the alcohol moiety to a leaving group such as a tosylate, mesylate, or halide, followed by displacement with an alkyl amine. Alternatively, 1-carbon homolgues of 5D-2 suchs as 5D-5 can be constructed via the cyano compound 5D-4.

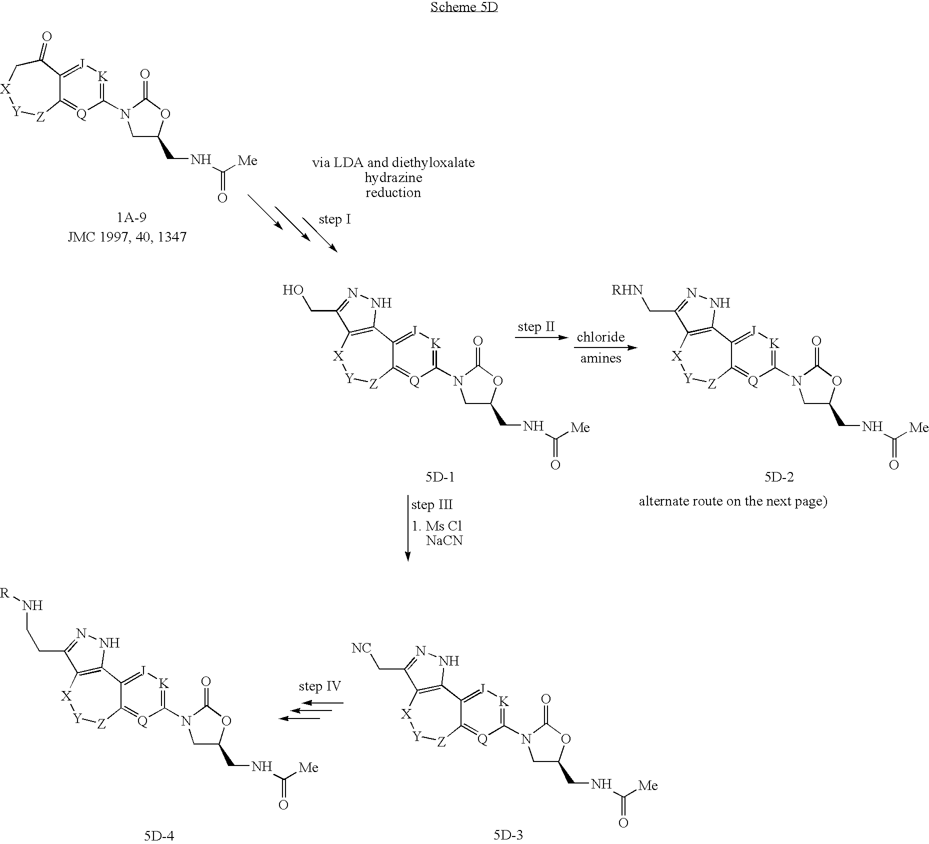

Scheme 5 E summarizes another strategy for the preparation of substituted diazinyl containing invention compounds. Thus compound 1A-9 is treated with dimethylcarbonate or nitilo acetic acid methyl ester in the presence of base to afford the β-ketoester 5E-1. Treatment of β-ketoester 5E-1 with hydrazine or a substituted hydrazine provides the diazinyl system 5E-2. Compound 5E-2 can be used as an intermediate in the preparation of other compounds, such as various ethers (via alkylations; see, e.g., 5E-3), or other systems via coupling procedures (see, e.g., 5E-4). Alternatively, compound 1A-9 can be converted to the β-ketoester 5E-1 and alkylated in situ to provide 5E-S. Compound 5E-S can be treated with hydrazine or a substituted hydrazine to give pyrazolone analogue 5E-6. Alternatively, 1A-9 can be converted to 5E-7 via esterification of the corresponding carboxylic acid (see Schemes 5B and 5C for the synthesis of the acid), converted to the diazine as provided above to give 5E-8, reduced to the hydroxymethyl compound 5E-9, and alkylated or coupled as provided for 5E-3 or 5E-4 to give 5E-10.

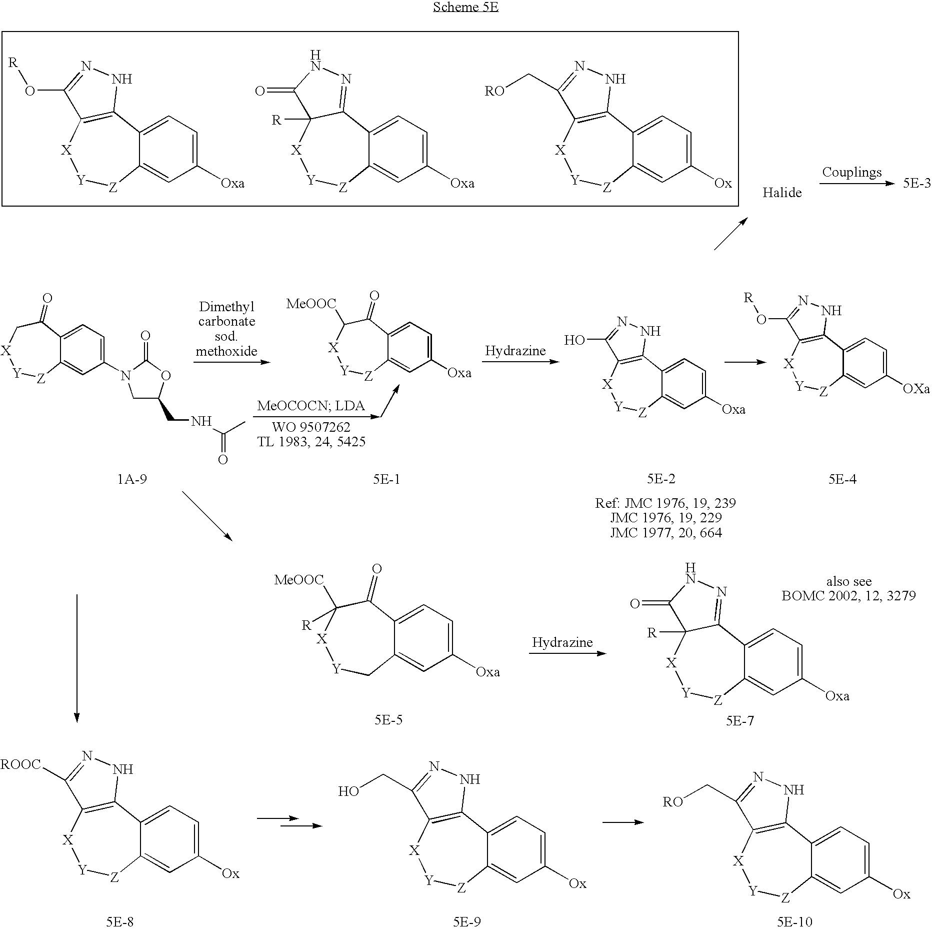

Scheme 5F highlights the synthesis of aminated diazinyl systems. Thus, compound 1A-9 is treated with carbon disulfide, and amine (such as piperizine, although the other, and methyl iodide in the presence of base to provide intermediate 5F-1. Compound 5F-1 is converted to diazinyl system 5F-2 via a series of reactions, including treatment with hydrazine or a substituted hydrazine; deprotection; acylation, followed by a carbon-nitogernt bond forming reaction such as sulfonylation, alkylation; or the like.

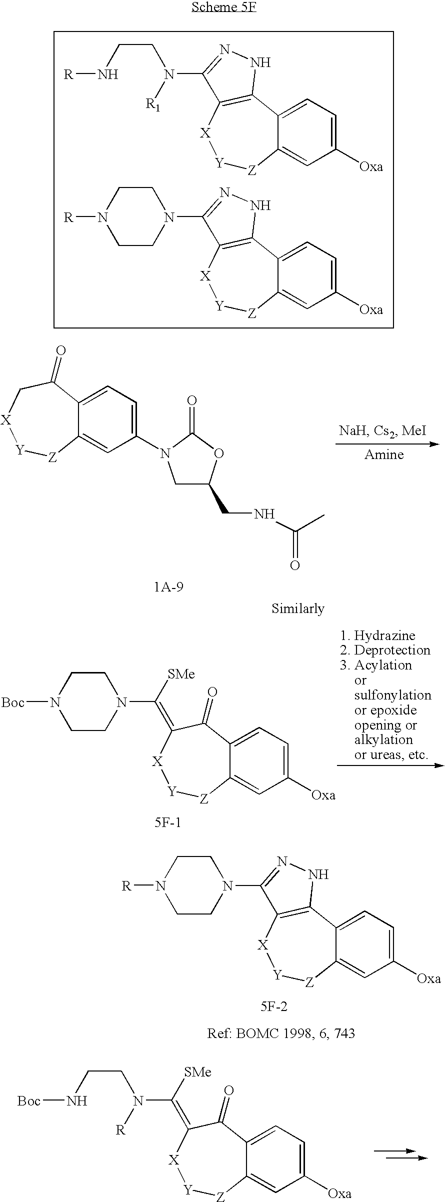

Scheme 5G provides an alternative approach to the synthesis of substituted diazinyl systems. Thus, compound 1A-9 is converted to the β-keto amide via treatment with a protected α, β, or γ-amino acid in the presence of carbonyl di-imidazole or the like to provide 5G-1. Treatment of 5G-1 with hydrazone or a substituted hydrazone as provided in earlier schemes gives rise to the target compound 5G-2, which may be derivatized further as provided in earlier schemes.

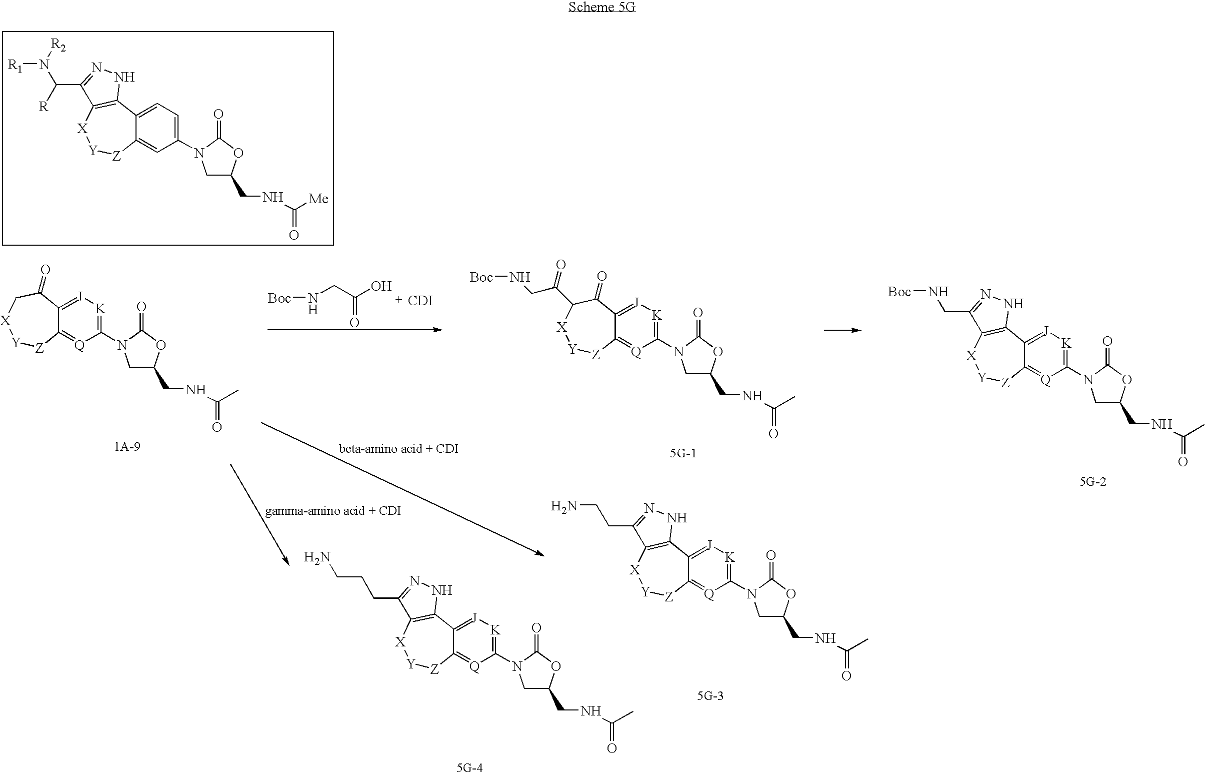

Scheme 5H provides another approach to the synthesis of substituted diazinyl systems. Thus, compound 1A-9 is converted to β-keto ester 5H-1 using methoxy acetic acid methyl ester. The diazinyl system 5H-2 is prepared as provided earlier using hydrazine or a substituted hydrazine. Conversion of 5H-2 to aldehyde 5H-3, followed by reductive amination, provides the target compound 5H-4. Alternatively, 5H-2 can be converted to the hydroxymethyl compound 5H-5, which may be alkylated or homolgated as indicated to give 5H-6 and 5H-8, respectively.

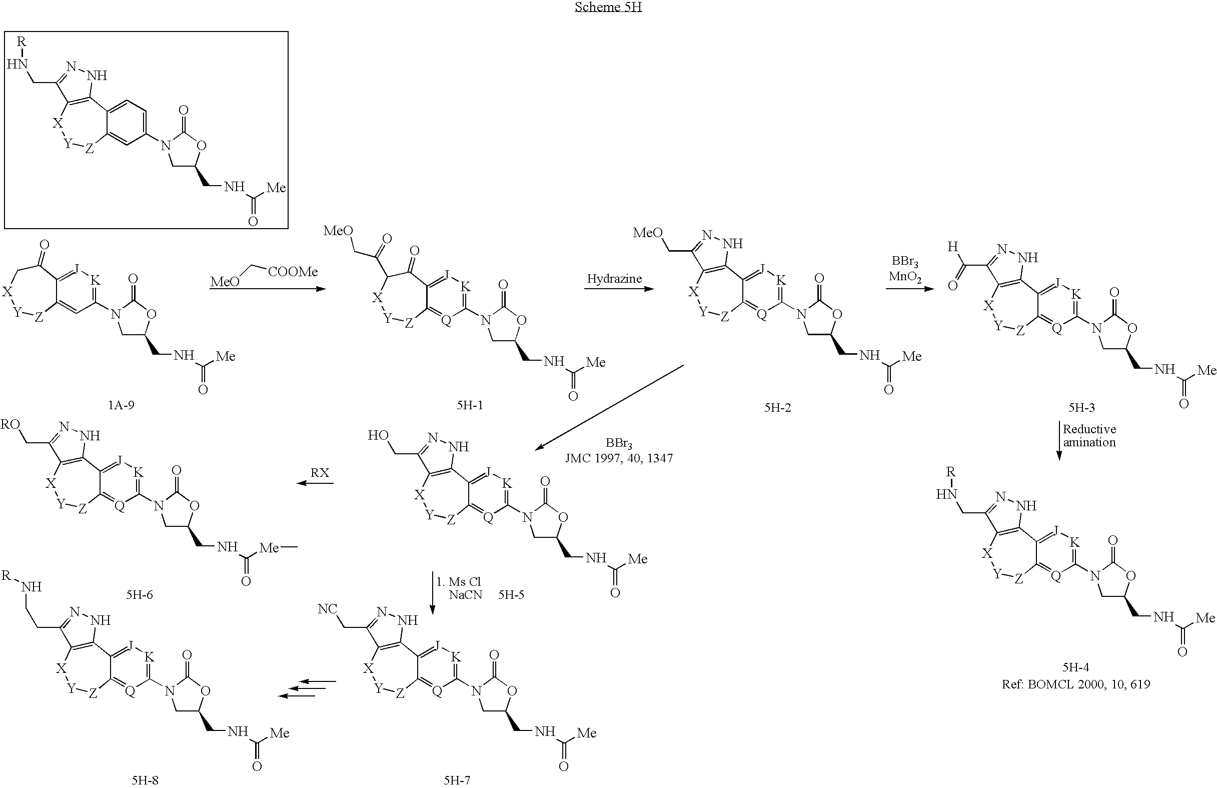

Scheme 5J provides an approach to other substituted diazinyl systems. Thus, compound 1A-9 is converted to the exo olefin 5J-1I via procedures well known to the skilled artisan. Epoxidation of 5J-1 provides 5J-2. Oxidative ring opening of the epoxide and treatment with hydrazine or a substituted hydrazine provides the target compound 5J-4.

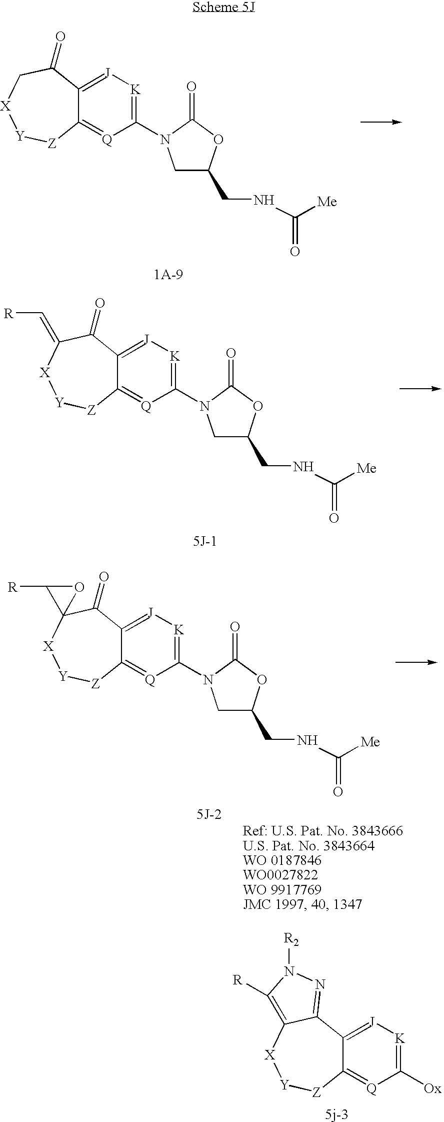

Scheme 6 provides an approach to diazines and isoxazoles via an α-cyano intermediate. Thus, compound 1A-9 undergoes bromination and subsequent cyanation to provide compound 6-1. Treatment of cyano compound 6-1 with hydrazine or hydroxylamine, or substituted variants thereof gives riste to diazine 6-2 or isoxazole 6-3.

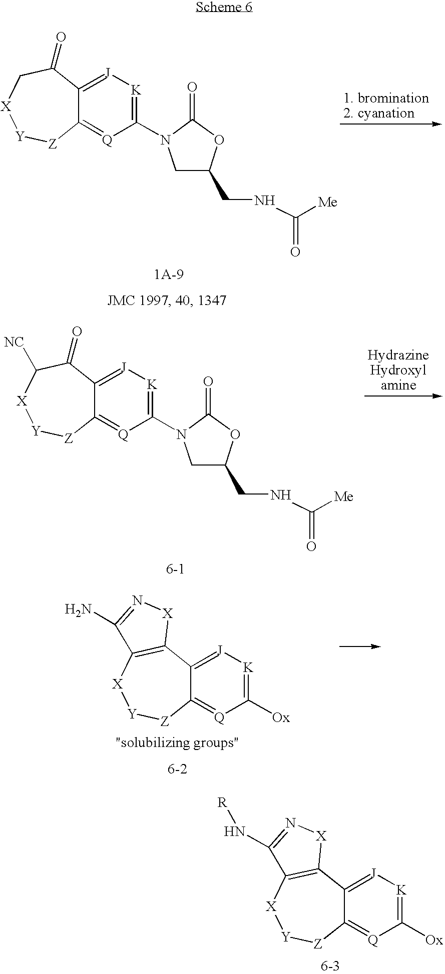

Scheme 7 provides an approach to pyrrole-containing systems, as well as furna-containing systems. The exo olefin 7-1 can be prepared as indicated in Scheme 5J. Conversion of 7-1 to a dicarbonyl compound 7-4, followed by base-mediated cyclization treatment, provides furan 7-5. Similarly, formation of the imine of 7-1, followed by cyclization, gives the corresponding pyrrole 7-6.

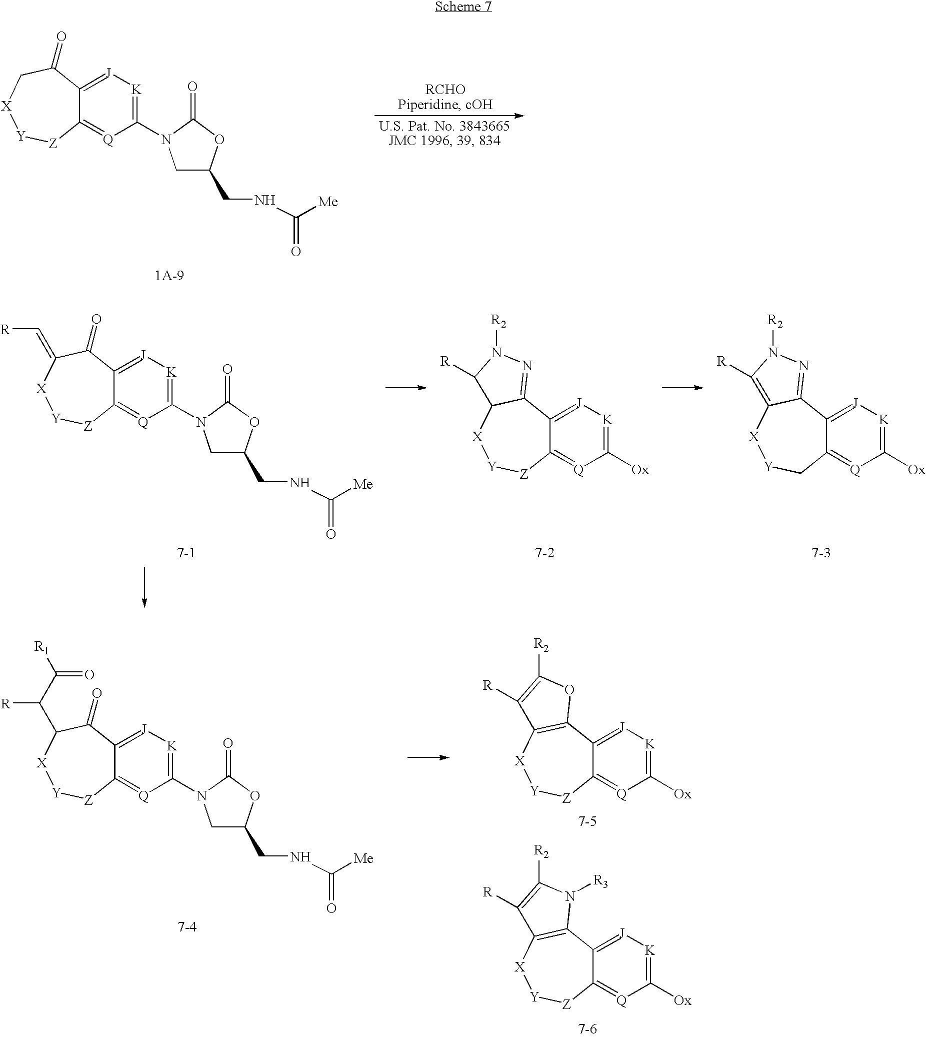

Scheme 8 provides approaches to thiazole- oxazole-, and imidazole-containing systems. Thus, bromination of compound 8-11 provides α-bromoketone 8-1. Treatment of 8-1 with a thiamide or thioacetic acid affords the requisite thiazole 8-2. Alternatively, treatment of 8-1 with a urea or an amine in the presence of hydroxylamine provides the corresponding imidazoles 8-3 and 8-4. The corresponding oxazole 8-5 can also be prepared via this general strategy,

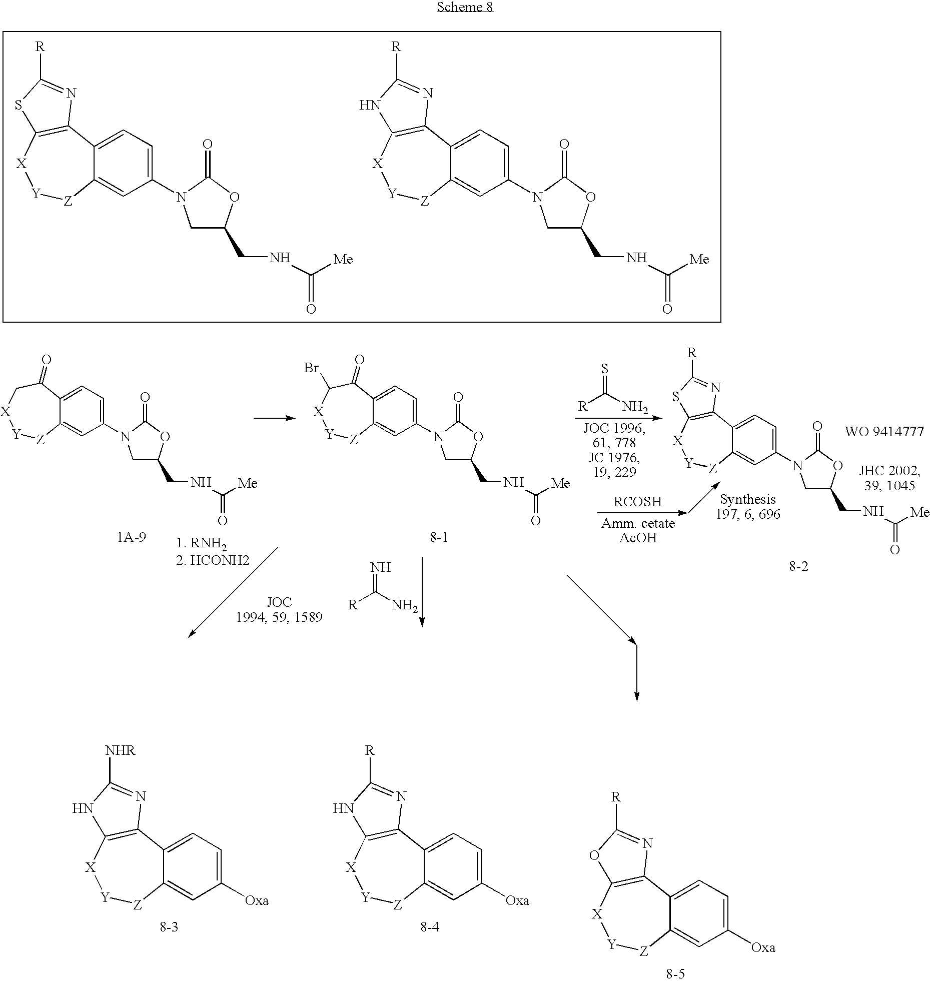

Scheme 9 summarzies an approach to isoxazole-containing systems. Thus, compound 1A-9 is treated with hydydroxylamine to provide the oxime 9-1. Treatment of 9-1 with base in the presence of an ester, followed by heating, provides the target isoxazole 9-2. 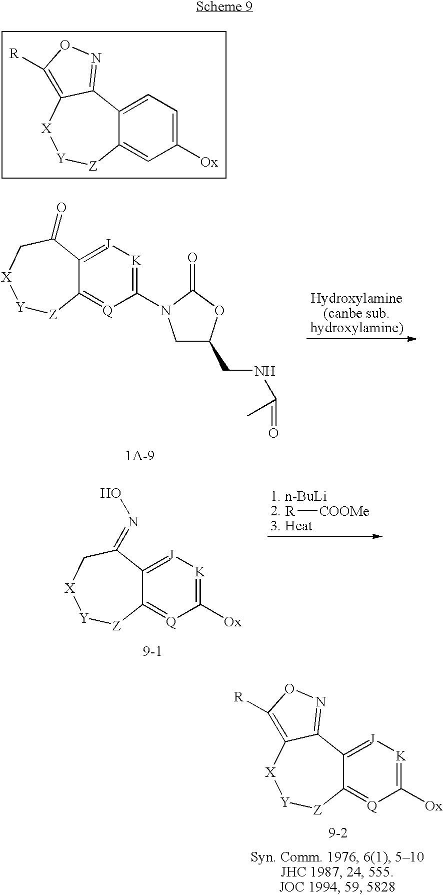
Pharmaceutical Formulations Center

The present invention also provides pharmaceutical compositions which comprise a bioactive invention compound or a salt such as a pharmaceutically acceptable salt thereof and optionally a pharmaceutically acceptable carrier. The compositions include those in a form adapted for oral, topical or parenteral use and can be used for the treatment of bacterial infection in mammals including humans.

The invention compounds, which are antibiotic compounds (also referred to herein as antimicrobial compounds) can be formulated for administration in any convenient way for use in human or veterinary medicine, by analogy with other bioactive agents which are antibiotics. Such methods are known in the art and are not described in detail herein.

The composition can be formulated for administration by any route known in the art, such as subdermal, by-inhalation, oral, topical or parenteral. The compositions may be in any form known in the art, including but not limited to tablets, capsules, powders, granules, lozenges, creams or liquid preparations, such as oral or sterile parenteral solutions or suspensions.

The topical formulations of the present invention can be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present, for example, from about 1% up to about 98% of the formulation. For example, they may form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods will known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% by weight, e.g., from about 10-60% by weight, of the active material, depending on the method of administration. Where the compositions comprise dosage units, each unit will contain, for example, from about 50-500 mg of the active ingredient. The dosage as employed for adult human treatment will range, for example, from about 100 to 3000 mg per day, for instance 1500 mg per day depending on the route and frequency of administration. Such a dosage corresponds to about 1.5 to 50 mg/kg per day. Suitably the dosage is, for example, from about 5 to 20 mg/kg per day.

The invention compounds disclosed herein can be used in a variety of pharmaceutical applications. In one embodiment, the compounds may be used as antimicrobial agents for the treatment of infectious disorders that are caused by microbial agents, such as bacteria.

In one embodiment, compositions, for treating or preventing infectious disorders are provided, comprising an oxazolidone compound as disclosed herein in combination with a pharmaceutically acceptable carrier.

In another embodiment, there is provided a dosage amount of an invention compound as disclosed herein in an effective amount for the treatment, prevention or alleviation of a disorder, such as an infectious disorder.

The invention compounds can be screened for activity against different microbial agents and appropriate dosages may be determined using methods available in the art.

The compounds may be used to treat a subject to treat, prevent, or reduce the severity of an infection. Subjects include animals, plants, blood products, cultures and surfaces such as those of medical or research equipment, such as glass, needles and tubing.

Antiinfective Activity Center

In one embodiment, methods of treating or preventing an infectious disorder in a subject, such as a human or other animal subject, are provided, by administering an effective amount of an invention compound as disclosed herein to the subject. In one embodiment, the compound is administered in a pharmaceutically acceptable form optionally in a pharmaceutically acceptable carrier. As used herein, an “infectious disorder” is any disorder characterized by the presence of a microbial infection, such as bacterial infections. Such infectious disorders include, for example central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. The compounds and compositions comprising the compounds can be administered by routes such as topically, locally or systemically. Systemic application includes any method of introducing the compound into the tissues of the body, e.g., intrathecal, epidural, intramuscular, transdermal, intravenous, intraperitoneal, subcutaneous, sublingual, rectal, and oral administration. The specific dosage of antimicrobial to be administered, as well as the duration of treatment, may be adjusted as needed.

The compounds of the invention may be used for the treatment or prevention of infectious disorders caused by a variety of bacterial organisms. Examples include Gram positive and Gram negative aerobic and anaerobic bacteria, including Staphylococci, for example S. aureus; Enterococci, for example E. faecalis; Streptococci, for example S. pneumoniae; Haemophilus, for example H. influenza; Moraxella, for example M. catarrhalis; and Escherichia, for example E. coli. Other examples include Mycobacteria, for example M. tuberculosis; intercellular microbes, for example Chlamydia and Rickettsiae; and Mycoplasma, for example M. pneumoniae.

The ability of a compound of the invention to inhibit bacterial growth, to demonstrate in vivo activity, and to enhance pharmacokinetics are demonstrated using pharmacological models that are well known to the art, for example, using models such as the tests described below.

Test A—Antibacterial Assays

The compounds of the present invention were tested against an assortment of Gram-negative and Gram-positive organisms using standard microtitration techniques (Cohen et. al., Antimicrob., 1985; 28: 766; Heifetz, et. al., Antimicrob., 1974; 6: 124). The results of the evaluation are shown in Tables 2A and B.

TABLE 2A

| | Gram Negative Bacteria | | |
|---|---|---|---|
| | MIC (μg/mL) | | |
| | | M. | |
| Structure/ | H. influenzae | catarrhalis | E. coli |
| Example No. | HI3542 | BC3534 | Tol C |
| 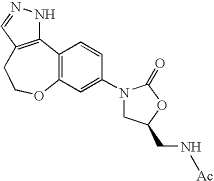 | 8 | 16 | >64 |
| 37 | 8 | 4 | >64 |
| 38 | 4 | no data | >64 |
| 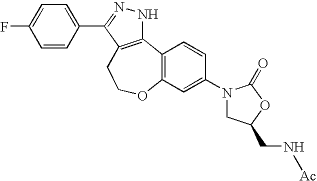 | | | |
| 22 | 4 | >64 | >64 |
| 39 | >64 | >64 | >64 |
| 40 | >64 | >64 | >64 |
| 41 | >64 | >64 | >64 |
| 42 | >64 | >64 | >64 |

TABLE 2B

| | Gram Positive Bacteria | | |
|---|---|---|---|
| | MIC (μg/mL) | | |
| Compound Structure or | E. faecalis | S. aureus | S pyogenes |
| Example No. | MGH-2 | UC-76 | C203 |
| 37 | 2 | 2 | 0.5 |
| 38 | 1 | 1 | 0.5 |
| 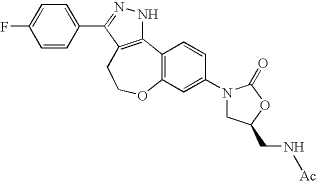 | 1 | 1 | 0.5 |
| 22 | 4 | 4 | 1 |
| 39 | 32 | >64 | 32 |
| 40 | >64 | >64 | 32 |
| 41 | >64 | >64 | 32 |
| 42 | 32 | 32 | 32 |

The compounds of the present invention were tested against E. coli transcription and translation (TnT) assay. The TnT assay is a cell free system that utilizes an E. coli S30 fraction and a “premix” to transcribe and translate the firefly luciferase gene from an exogenously supplied plasmid DNA. The amount of luciferase produced is measured by observing the luminescence produced after addition of a luciferase assay reagent. The TnT assay reagents, including the luciferase reporter plasmid pBESTluc, were purchased from Promega Corporation. The protocol was based upon the manufacturer's instructions (Promega Technical Bulletin number 92 “E. coli S30 Extract System for Circular DNA”). Luciferase assay reagent (LucLite Plus) was purchased from Packard Biosciences.

The assay was conducted in white, flat-bottomed, polystyrene 96-well plates. Each well contained S30, premix, amino acids, compound and DNA in a total volume of 35 microliters. The reactions were allowed to incubate at room temperature for 20 minutes, then quenched with 35 microliters of LucLite Plus. The plate was then sealed with an aluminum foil lid and allowed to mix on a plate shaker for five minutes. The plate was then uncovered and read on the LJL Analyst using the standard luminescence protocol. The assay can also be read with a Perkin-Elmer Microbeta Trilux using a 1450-105 96 well plate cassette utilizing a protocol with a 10 second counting time, no background correction, and upper PMT usage. The results of the evaluation are shown in Table 2C.

TABLE 2C

| Compound Structure or | E. coli TnT Assay |
|---|---|
| Example No | MIC (μ/mL) |
| 37 | 3.1 |
| 38 | 2.5 |
| 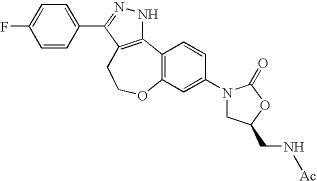 | 2.6 |
| 39 | 35 |

The following examples are provided to illustrate but not limit the claimed invention.

### General Procedures

The following general procedures were employed in preparing the invention compounds and are referenced as accordingly in the Example Section.

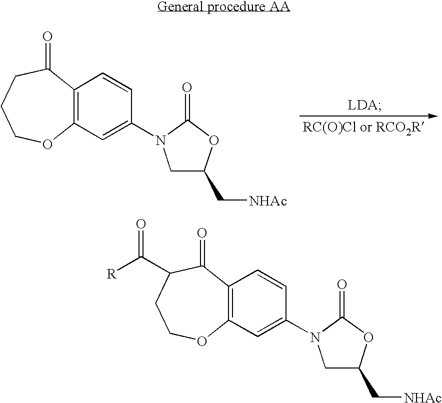

1,3-Diketone formation #1: The starting ketone was dissolved in dry THF under nitrogen atmosphere, and cooled to −78° C. in acetone/dry ice bath. Lithium diisopropylamine (LDA, 2M, 2.0-2.4 equiv.) was added and the resulting mixture stirred at −78° C. for approximately 20 minutes. The corresponding acid chloride or ester (neat, 1.0-1.5 equiv.) was added and the mixture was allowed to stir at −78° C. for 15 to 20 minutes, followed by stirring at 0° C. The mixture was then allowed to warm to room temperature overnight. The reaction was quenched with saturated NH₄Cl or 0.5 N HCl, followed by EtOAc or dichloromethane extraction. The organic phase was washed with brine, dried over MgSO₄, filtered and concentrated. The isolated residue was subjected to silica gel flash chromatography to afford the desired compound unless otherwise noted.

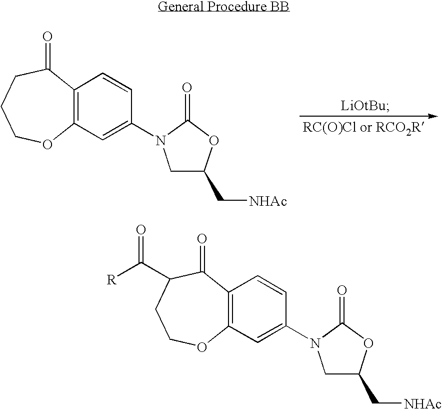

1,3-Diketone formation #2: To the starting ketone dissolved in THF was added lithium t-butoxide (1 M in hexanes, 2.1-3.1 equiv.) followed by addition of the corresponding acid chloride or ester (1.1-1.2 equiv.). The resulting mixture was heated at reflux overnight. Either HCl (0.5 N) or saturated NH₄Cl was then added, followed by EtOAc or dichloromethane extraction. The organic phase was washed with brine, dried over magnesium sulfate, filtered, and concentrated. The resulting residue was subjected to flash silica gel chromatography to afford the desired product unless otherwise noted.

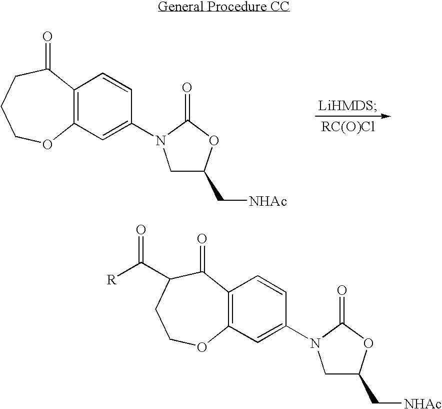

1,3-Diketone formation #3: The starting ketone was dissolved in THF, cooled to 0° C., and lithium bis(trimethylsilyl)amide (LiHMDS, 1 M in THF, 2.0-3.15 equiv.) was added dropwise via syringe. The reaction mixture was then stirred approximately 30 minutes, after which the corresponding acid chloride (1.0-1.2 equiv.) was added either as a solid or was added dropwise as a solution in THF. The resulting mixture was stirred at 0° C. and then allowed to warm slowly to room temperature overnight. Either HCl (0.5 N) or saturated NH₄Cl was then added, followed by EtOAc or dichloromethane extraction. The organic phase was washed with brine, dried over magnesium sulfate, filtered, and concentrated. The resulting residue was subjected to flash silica gel chromatography to afford the desired product unless otherwise noted.

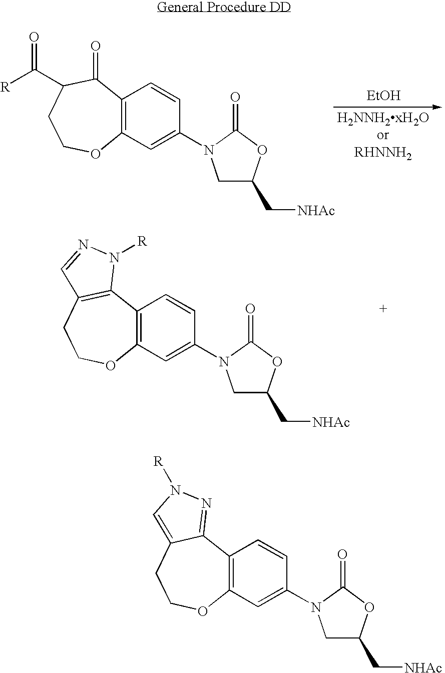

Pyrazole formation: The starting 1,3-diketone was placed in EtOH and to this was added hydrazine hydrate (2.5-5.0 equiv.) or an appropriately substituted hydrazine (2.5-4.0 equiv.). If a slurry resulted, the reaction flask was sometimes heated with warm water (approximately 50-60° C.) until all solids dissolved. The slurry or solution was then stirred at room temperature for 24-72 hours. The solvent was then removed in vacuo and the resulting residue was subjected to flash silica gel chromatography to afford the desired product unless otherwise noted.

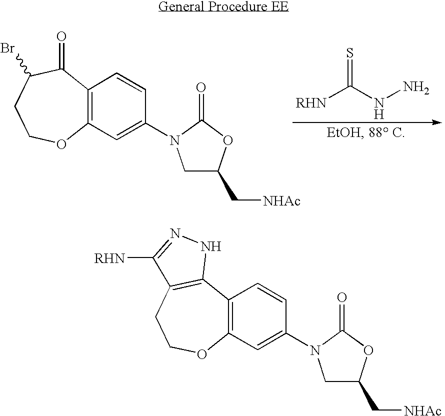

Bromoketone (1 mmol), the appropriate thiosemicarbazide RHNCSNHNH₂ (1 mmol), and 10 mL of absolute EtOH were heated to 88° C. Upon completion of the reaction, the solution was cooled to room temperature, treated with 4 mL of saturated. NaHCO₃, and concentrated in vacuo. The aqueous layer was extracted with several portions of dichloromethane or dichloromethane/MeOH. The combined organic layers were dried over Na₂SO₄, filtered, concentrated in vacuo, and then purified by silica gel chromatography.

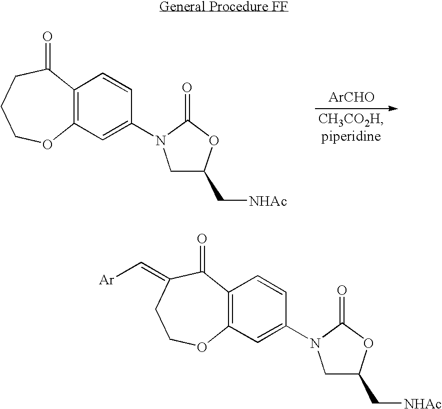

α,β-unsaturated ketone formation: To the ketone (1 equiv.) was added the aromatic aldehyde (4 equiv.). Acetic acid and piperidine were then added. The reaction was heated to 80-100° C. for 4-12 hours. The reaction was cooled to room temperature and diluted with dichloromethane. The organic layer was washed with water, potassium carbonate solution, dilute hydrochloric acid, and then brine. The organic layer was dried over MgSO₄, filtered, concentrated in vacuo and then purified by silica gel chromatography.

### EXAMPLE 1

### N-[3-(2-Methyl-4,5-dihydro-3H-6-oxa-1,3-diaza-benzo[e]azulen-8-yl)-2-oxo-oxazolidin-5-ylmethyl]-acetamide

### N-[3-(2-Trifluoromethyl-4,5-dihydro-3H-6-oxa-1,3-diaza-benzo[e]azulen-8-yl)-2-oxo-oxazolidin-5-ylmethyl]-acetamide; and

### N-[3-(2-Phenyl-4,5-dihydro-3H-6-oxa-1,3-diaza-benzo[e]azulen-8-yl)-2-oxo-oxazolidin-5-ylmethyl]-acetamide

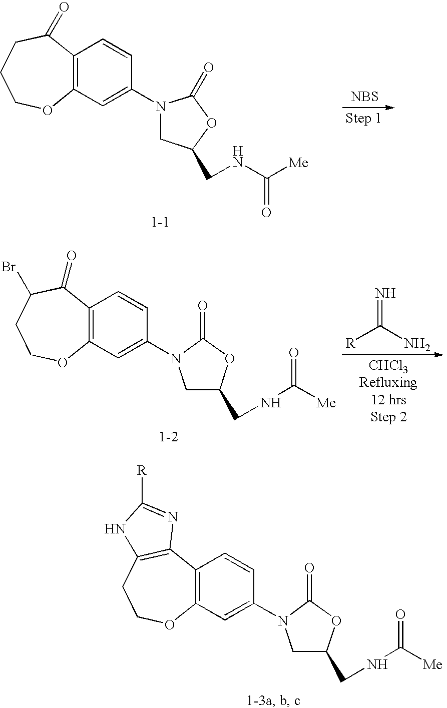

### N-[3-(4-Bromo-5-oxo-2,3,4,5-tetrahydro-benzo[b]oxepin-8-yl)-2-oxo-oxazolidin-5-ylmethyl]-acetamide 1-2 (Step 1)

The title compound was prepared by bromination of N-[2-Oxo-3-(5-oxo-2,3,4,5-tetrahydro-benzo[b]oxepin-8-yl)-oxazolidin-5-ylmethyl]-acetamide using N-bromosuccinimide. NMR (CDCl₃, 400 MHz): δ 2.00 (s, 3H), 3.29 (t, J=5.1 Hz, 2H), 3.66 (m, 2H), 3.79 (t, J=7.1 Hz, 1H), 4.06 (m, 1H), 4.34 (m, 2H), 4.80 (m, 1H), 6.28 (br, 1H), 7.20 (s, 1H), 7.30 (m, 1H), 7.71 (d, J=8.7 Hz, 1H). M.S. m/z 397 (M+1),

### N-[3-(2-Methyl-4,5-dihydro-3H-6-oxa-1,3-diaza-benzo[e]azulen-8-yl)-2-oxo-oxazolidin-5-ylmethyl]-acetamide (1-3a) (Step 2)

To a solution of N-[3-(4-Bromo-5-oxo-2,3,4,5-tetrahydro-benzo[b]oxepin-8-yl)-2-oxo-oxazolidin-5-ylmethyl]-acetamide (396 mg, 1.0 mmol) in 10 mL of chloroform was added the free base of acetamidine (232 mg, 4.0 mmol). The resulting solution was heated to reflux overnight. Ethyl acetate (15 mL) was added to the reaction mixture, and the organic layer was washed with saturated sodium bicarbonate solution and dried over sodium sulfate. The crude product was purified by silica gel chromatography to afford 78 mg (22%) of the title compound. MS m/z 357 (M+1). NMR (CDCl₃, 400 MHz: δ 2.00 (s, 3H), 3.10 (m, 2H), 3.47 (s, 3H), 3.75 (m, 2H), 3.74 (t, 2H), 4.03 (m, 2H), 4.29 (m, 2H), 4.70 (m, 1H), 6.71 (br, 1H), 7.21(m, 1H), 7.29 (dd, 2H), 8.04 (d, 1H).

By using a correspondingly substituted acetamidine and the common bromide intermediate, N-[3-(2-Methyl-4,5-dihydro-3H-6-oxa-1,3-diaza-benzo[e]azulen-8-yl)-2-oxo-oxazolidin-5-ylmethyl]-acetamide and N-[3-(2-Trifluoromethyl-4,5-dihydro-3H-6-oxa-1,3-diaza-benzo[e]azulen-8-yl)-2-oxo-oxazolidin-5-ylmethyl]-acetamide (CE-127889 and CE-127890) were made using the above procedure.

### N-[3-(2-Methyl-4,5-dihydro-3H-6-oxa-1,3-diaza-benzo[e]azulen-8-yl)-2-oxo-oxazolidin-5-ylmethyl]-acetamide (1-3a)

M.S. m/z 411 (M+1). NMR (CD₃CN, 400 MHz): δ 1.86 (s, 3H), 3.15 (m, 2H), 3.49 (m, 2H), 3.74 (t, 2H), 4.04 (m, 2H), 4.27 (m, 2H), 4.65 (m, 1H), 6.70 (br, 1H), 7.19 (m, 1H), 7.29 (dd, 2H), 8.02 (d, 1H).

### N-[3-(2-Trifluoromethyl-4,5-dihydro-3H-6-oxa-1,3-diaza-benzo[e]azulen-8-yl)-2-oxo-oxazolidin-5-ylmethyl]-acetamide (1-3b)

M.S. m/z 419 (M+1). NMR (CD₃OD, 400 MHz): δ 1.95 (s, 3H), 3.17 (m, 2H), 3.28 (t, J=5.9 Hz, 2H), 3.54 (m, 2H), 3.81 (m, 2H), 4.11 (t, J=9.1 Hz, 2H), 4.30 (t, J=5.9 Hz, 2H), 7.26 (m, 2H), 7.38 (m, 1H), 7.41(m, 2H), 7.90 (d, J=9.1 Hz, 1H), 8.01 (d, J=9.0 Hz, 1H).

### EXAMPLE 2

### N-[3-(2-Methyl-4,5-dihydro-6-oxa-3-thia-1-aza-benzo[e]azulen-8-yl)-2-oxo-oxazolidin-5-ylmethyl]-acetamide a (2-1a)

### N-[3-(2-Amino-4,5-dihydro-6-oxa-3-thia-1-aza-benzo[e]azulen-8-yl)-2-oxo-oxazolidin-5-ylmethyl]-acetamide (2-1b)

### N-[3-(2-Phenyl-4,5-dihydro-6-oxa-3-thia-1-aza-benzo[e]azulen-8-yl)-2-oxo-oxazolidin-5-ylmethyl]-acetamide (2-1c)

### N-[3-(2-(4-Pyridyl)-4,5-dihydro-6-oxa-3-thia-l -aza-benzo[e]azulen-8-yl)-2-oxo-oxazolidin-5-ylmethyl]-acetamide (2-1d); and

### N-[3-(2-(4-methoxyphenyl)-4,5-dihydro-6-oxa-3-thia-1-aza-benzo[e]azulen-8-yl)-2-oxo-oxazolidin-5-ylmethyl]-acetamide (2-1e)

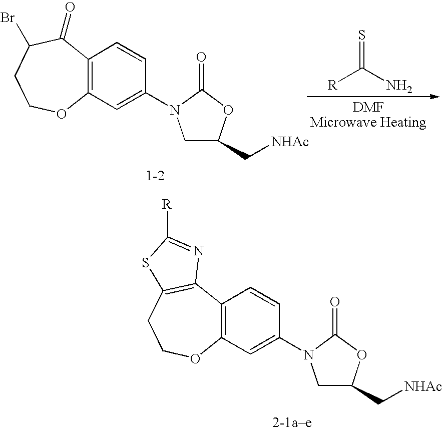

### N-[3-(2-Methyl-4,5-dihydro-6-oxa-3-thia-1-aza-benzo[e]azulen-8-yl)-2-oxo-oxazolidin-5-ylmethyl]-acetamide

To a solution of N-[3-(4-Bromo-5-oxo-2,3,4,5-tetrahydro-benzo[b]oxepin-8-yl)-2-oxo-oxazolidin-5-ylmethyl]-acetamide 1-2 (Example 1)(396mg, 1.0 mmol) in 2 mL DMF was added thioacetamide (375 mg, 5.0 mmol) as a solid. The resulting solution was heated to 150° C. for 15 minutes by using a microwave reactor. Ethyl acetate (15 mL) was then added to the reaction mixture. The organic layer was washed with saturated sodium bicarbonate solution, then dried over sodium sulfate. The crude product was purified by silica gel chromatography to afford 70 mg (19%) of the title compound. M.S. m/z 374 (M+1). NMR (CDCl₃, 400 MHz): δ 2.06 (s,3H ), 2.72 (s, 3H), 3.72 (m, 3H), 4.10 (m,1H), 4.40 (m, 2H), 4.80 (m, 1H), 6.20 (t, J=6.8 Hz, 1H), 7.29 (m,1H), 7.84 (d, J=9.0 Hz, 1H), 8.45 (d, J=8.4 Hz, 1H).

By using a correspondingly substituted thioacetamide and the common bromide intermediate, N-[3-(2-Amino-4,5-dihydro-6-oxa-3-thia-1-aza-benzo[e]azulen-8-yl)-2-oxo-oxazolidin-5-ylmethyl]-acetamide, N-[3-(2-Phenyl-4,5-dihydro-6-oxa-3-thia-1-aza-benzo[e]azulen-8-yl)-2-oxo-oxazolidin-5-ylmethyl]-acetamide; N-[3-(2-(4-Pyridyl)-4,5-dihydro-6-oxa-3-thia-1-aza-benzo[e]azulen-8-yl)-2-oxo-oxazolidin-5-ylmethyl]-acetamide, and N-[3-(2-(4-methoxyphenyl)-4,5-dihydro-6-oxa-3-thia-1-aza-benzo[e]azulen-8-yl)-2-oxo-oxazolidin-5-ylmethyl]-acetamide were made using the above procedure.

### N-[3-(2-Amino-4,5-dihydro-6-oxa-3-thia-1-aza-benzo[e]azulen-8-yl)-2-oxo-oxazolidin-5-ylmethyl]-acetamide (2-1a)

M.S. m/z 375 (M+1). NMR (CD₃OD, 400 MHz): δ 1.95 (s, 3H), 3.08 (t, J=5.4 Hz, 1H), 3.54 (m, 2H), 3.80 (m, 1H), 4.13 (t, J=9.1 Hz, 1H), 4.27 (t, J=5.0 Hz, 1H), 4.76 (m, 1H), 7.23 (t, J=2.1 Hz, 1H),8.08 (d, J=8.7 Hz, 1H), 8.43 (m, 1H).

### N-[3-(2-Phenyl-4,5-dihydro-6-oxa-3-thia-l -aza-benzo[e]azulen-8-yl)-2-oxo-oxazolidin-5-ylmethyl]-acetamide (2-1b)

M.S. m/z 436 (M+1). NMR (CDCl₃, 400 MHz): δ 2.00 (s, 3H), 3.36 (m, 2H), 3.60 (m, 1H), 3.76 (m, 2H), 4.08 (m, 1H), 4.38 (t, J=5.4 Hz, 1H), 4.78 (m, 1H), 6.04 (t, J=5.8 Hz, 1H), 7.25 (m, 11H), 7.31 (dd, J=9.1, 2.5 Hz ,1H), 7.43 (m, 3H), 7.96 (m, 2H), 8.60(d, J=9.1 Hz, 1H).

### N-[3-(2-(4-Pyridyl)-4,5-dihydro-6-oxa-3-thia-1-aza-benzo[e]azulen-8-yl)-2-oxo-oxazolidin-5-ylmethyl]-acetamide (2-1c)

M.S. m/z 437 (M+1). NMR (CDCl₃, 400 MHz): δ 2.02 (s, 3H), 3.38 (m, 2H), 3.68(m, 2H), 3.80 (dd, 1H, J=7.1Hz), 4.08(t, J=9.1 Hz, 1H), 4.37(tm, 2H), 4.78 (m, 1H), 6.15 (t, J=6.6 Hz 1H), 7.25 (m, 2H), 7.32 (dd, J=8.7, 2.1 Hz, 1H), 7.90 (d, J=4.2 Hz, 2H), 8.55 (m,1H), 8.70 (m, 2H).

### N-[3-(2-(4-methoxyphenyl)-4,5-dihydro-6-oxa-3-thia-1-aza-benzo[e]azulen-8-yl)-2-oxo-oxazolidin-5-ylmethyl]-acetamide (2-1d)

M.S. m/z 466 (M+1). NMR (CDCl₃, 400 MHz): δ 2.01 (s, 3H), 3.32 (t, J=4.6 Hz, 1H), 3.62(m, 1H), 3.71 (m, 1H), 3.85 (s, 3H), 4.07(t, J=9.1 Hz, 1H), 4.37(t, J=5.4 Hz, 1H), 4.79 (m, 1H), 6.12 (t, J=5.8 Hz, 1H), 6.95 (m, 1H), 7.89 (d, J=8.7 Hz, 1H),8.58 (m, 1H).

### EXAMPLE 3

### N-[3-(4,5-Dihydro-1H-6-oxa-1,2-diaza-benzo[e]azulen-8-yl)-2-oxo-oxazolidin-5-ylmethyl]-acetamide (3-2a)

### N-[3-(1-Methyl-4,5-dihydro-1H-6-oxa-1,2-diaza-benzo[e]azulen-8-yl)-2-oxo-oxazolidin-5-ylmethyl]-acetamide (3-2b)

### N-[3-(1-(1-1-1-Triflouroethyl-4,5-dihydro-1H-6-oxa-1,2-diaza-benzo[e]azulen-8-yl)-2-oxo-oxazolidin-5-ylmethyl]-acetamide (3-2c)

### N-[3-(1-(1-Hydroxyethyl)-4,5-dihydro-1H-6-oxa-1,2-diaza-benzo[e]azulen-8-yl)-2-oxo-oxazolidin-5-ylmethyl]-acetamide (3-2d)

### N-[3-(1-(3-Chlorophenyl)-4,5-dihydro-1H-6-oxa-1,2-diaza-benzo[e]azulen-8-yl)-2-oxo-oxazolidin-5-ylmethyl]-acetamide (3-2e); and

### N-[3-(2-Cyanomethyl-4,5-dihydro-2H-6-oxa-1,2-diaza-benzo[e]azulen-8-yl)-2-oxo-oxazolidin-5-ylmethyl]-acetamide (3-2f)

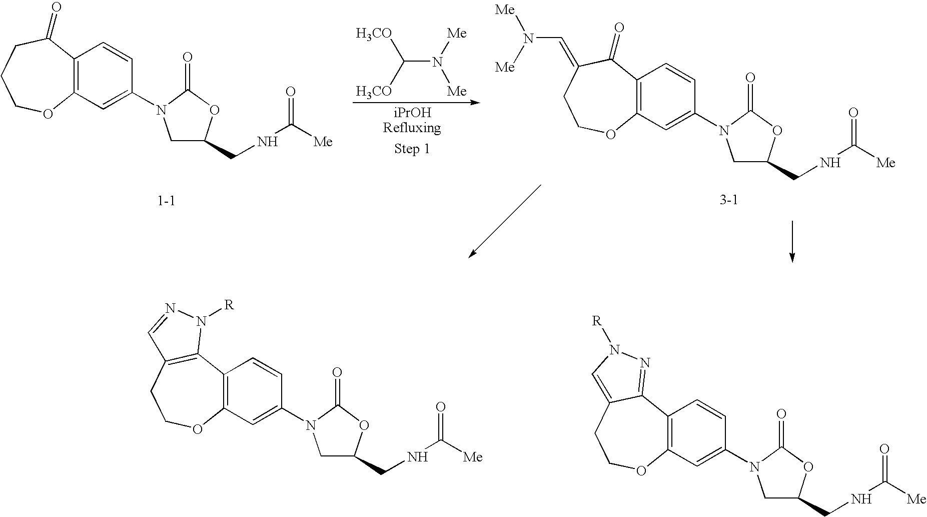

### N-[3-(4-Dimethylaminomethylene-5-oxo-2,3,4,5-tetrahydro-benzo[b]oxepin-8-yl)-2-oxo-oxazolidin-5-ylmethyl]-acetamide (3-1) (Step 1)

To a solution of N-[2-Oxo-3-(5-oxo-2,3,4,5-tetrahydro-benzo[b]oxepin-8-yl)-oxazolidin-5-ylmethyl]-acetamide (530 mg, 1.67 mmol) in 10 mL of ethanol (or other alcoholic solvent) was added N,N-dimethylformamide dimethyl acetal (1.33 mL, 10.02 mmol). The resulting solution was heated to reflux under N₂ for 12 hours. Ethanol and excess N,N-dimethylformamide dimethyl acetal were removed under reduced pressure and the crude product obtained was purified by silica gel chromatography (MeOH/CH₂Cl₂). The pure product (522 mg) was obtained as a solid (84% yield). M.S. m/z 374 (M+1). NMR (CDCl₃, 400 MHz): δ 1.98 (s, 3H), 2.70 (t, J=5.8 Hz, 1H), 3.10 (s, 6H), 3.60 (m, 2H), 3.73 (m, 1H), 4.00 (t, J=9.1 Hz, 1H), 4.22 (t, J=5.8 Hz, 1H), 4.74 (m, 1H), 6.64 (m, 1H), 7.22 (m, 2H), 7.68 (m, 2H).

### N-[3-(4,5-Dihydro-1H-6-oxa-1,2-diaza-benzo[e]azulen-8-yl)-2-oxo-oxazolidin-5-ylmethyl]-acetamide (3-2a) (Step 2)

To a solution of N-[3-(4-Dimethylaminomethylene-5-oxo-2,3,4,5-tetrahydro-benzo[b]oxepin-8-yl)-2-oxo-oxazolidin-5-ylmethyl]-acetamide (373 mg, 1.0 mmol) in 10 mL of ethanol was added hydrazine hydrate (93 μl, 4.0 mmol). The resulting solution was stirred at room temperature for 48 h. Ethanol and excess hydrazine hydrate were removed under reduced pressure to afford a cake. The pure product (234 mg) was obtained after purification by silica gel chromatography (80% yield). M.S. m/z 343 (M+1). NMR (CDCl₃, 400 MHz): δ 2.13 (s, 3H), 3.03 (t, J=4.6 Hz, 2H), 3.49 (t, J=5.8 Hz, 2H), 3.73 (dd, 2H), 4.05 (dd, 2H), 4.26 (m, 2H), 4.69 (m, 1H), 6.68 (br, 1H), 7.18 (d, J=2.1 Hz, 1H), 7.29 (m, 1H), 7.47 (s, 1H), 8.06 (br, 1H).

By using a correspondingly substituted hydrazine and the common intermediate, N-[3-(4-Dimethylaminomethylene-5-oxo-2,3,4,5-tetrahydro-benzo[b]oxepin-8-yl)-2-oxo-oxazolidin-5-ylmethyl]-acetamide, N-[3-(1-(1-1-1-Triflouroethyl-4,5-dihydro-1H-6-oxa-1,2-diaza-benzo[e]azulen-8-yl)-2-oxo-oxazolidin-5-ylmethyl]-acetamide, N-[3-(1-(1-Hydroxyethyl)-4,5-dihydro-1H-6-oxa-1,2-diaza-benzo[e]azulen-8-yl)-2-oxo-oxazolidin-5-ylmethyl]-acetamide, and N-[3-(1-(3-Chlorophenyl)-4,5-dihydro-1H-6-oxa-1,2-diaza-benzo[e]azulen-8-yl)-2-oxo-oxazolidin-5-ylmethyl]-acetamide were made using the above procedure.

### N-[3-(1-Methyl-4,5-dihydro-1H-6-oxa-1,2-diaza-benzo[e]azulen-8-yl)-2-oxo-oxazolidin-5-ylmethyl]-acetamide (3-2b)

M.S. m/z 357 (M+1). NMR (CDCl₃, 400 MHz): δ 1.99 (s, 3H), 3.00 (t, J=5.0 Hz, 2H), 3.57 (m, 1H), 3.73 (dd, J=9.1, 7.1 Hz, 2H), 3.88 (s, 3H), 4.00 (m, 2H), 4.24(m, 2H), 4.74 (m, 1H), 6.47(t, J=6.2 Hz, 1H), 7.15 (d, J=2.5 Hz, 1H), 7.21 (m, 1H), 7.47 (s, 1H), 8.18 (d, J=9.1 Hz, 1H).

### N-[3-(1-(1-1-1-Triflouroethyl-4,5-dihydro-1H-6-oxa-1,2-diaza-benzo[e]azulen-8-yl)-2-oxo-oxazolidin-5-ylmethyl]-acetamide (3-2c)

M.S. m/z 425 (M+1). NMR (CD₃OD, 400 MHz): δ 1.95 (s, 3H), 3.05 (t, J=5.0 Hz, 2H), 3.54 (d, J=5.0 Hz, 2H), 3.80 (dd, J=9.5, 6.6 Hz, 1H), 4.13 (t, J=9.1 Hz, 1H), 4.25 (t, J=5.4 Hz, 2H), 4.24(m, 2H), 4.74 (m, 1H), 6.47(t, J=6.2 Hz, 1H), 7.15 (d, J=2.5 Hz, 1H), 7.21 (m, 1H), 7.47 (s, 1H), 8.18 (d, J=9.1 Hz, 1H).

### N-[3-(1-(1-Hydroxyethyl)-4,5-dihydro-1H-6-oxa-1,2-diaza-benzo[e]azulen-8-yl)-2-oxo-oxazolidin-5-ylmethyl]-acetamide (3-2d)

M.S. m/z 387 (M+1). NMR (CD₃CN, 400MHz): δ 1.86 (s, 3H), 2.98 (t, J=5.0 Hz, 2H), 3.49 (m, 3H), 3.70 (m, 2H), 3.84 (t, J=5.0 Hz, 1H) 3.92 (m, 1H), 4.0 (m, 1H), 4.14 (t, J=5.4 Hz, 1H), 4.23 (m, 2H), 4.68 (m, 1H), 6.81 (t, J=5.0 Hz, 1H), 7.13 (d, J=2.5 Hz, 1H), 7.43 (d, J=3.7 Hz, 1H), 8.14 (d, J=9.0 Hz, 1H).

### N-[3-(1-(3-Chlorophenyl)-4,5-dihydro-1H-6-oxa-1,2-diaza-benzo[e]azulen-8-yl)-2-oxo-oxazolidin-5-ylmethyl]-acetamide (3-2e)

M.S. m/z 454 (M+1). NMR (CD₃CN, 400MHz): δ 1.85 (s, 3H), 3.0 (t, J=6.2 Hz, 2H), 3.47 (t, J=5.0 Hz, 2H), 3.68 (m, 1H), 3.98 (t, J=9.1 Hz, 1H), 4.34(t, J=6.2 Hz, 2H), 4.66 (m, 1H), 6.73 (m, 1H), 6.78 (d, J=9.1 Hz, 1H), 7.03 (dd, J=8.7, 2.5 Hz, 1H), 7.20 (m, 1H), 7.36 (m, 4H), 7.62 (s, 1H).

### N-[3-(2-Cyanomethyl-4,5-dihydro-2H-6-oxa-1,2-diaza-benzo[e]azulen-8-yl)-2-oxo-oxazolidin-5-ylmethyl]-acetamide (3-2f)

To a solution of N-[3-(4,5-Dihydro-1H-6-oxa-1,2-diaza-benzo[e]azulen-8-yl)-2-oxo-oxazolidin-5-ylmethyl]-acetamide (CP-947156, 342 mg, 1.0 mmol) in 5 mL of DMF at 0° C. was added NaH (26.4 mg, 1.1 mmol) as a solid. The resulting suspension was stirred under N₂ for 30 minutes. BrCH₂CN (70 μL, 1.0 mmol) was added, and the mixture was stirred at room temperature for another 4 h. Water (10 mL) was added to the reaction mixture, and the aqueous layer was extracted with EtOAc (3×50 mL). The organic layers were combined, dried over Na₂SO₄ and evaporated under reduced pressure to afford the crude product. After silica gel chromatography, 120 mg of pure product was recovered as a solid (32% yield).

M.S. m/z 382 (M+1). NMR (CD₃CN, 400 MHz) δ1.86 (s, 3H), 3.02 (t, J=5.0 Hz, 2H), 3.49 (t, J=5.0 Hz, 2H), 3.73 (dd, J=9.1 Hz, 6.2 Hz, 1H), 4.05 (t, J=9.1 Hz, 1H), 4.25 (t, J=4.6 Hz, 2H), 4.69 (m, 1H), 6.70 (br, 1H), 7.18 (d, J=2.5 Hz, 1H), 7.29 (dd, J=8.7 Hz, 2H), 3.02 (t, J=5.0 Hz, 2H), 3.02 (t, J=5.0 Hz, 2H), 3.02 (t, J=5.0 Hz, 2H), 3.02 (t, J=5.0, 2.5 Hz, 1H), 7.47 (s, 1H), 8.05 (d, J=8.7 Hz, 1H).

### EXAMPLE 4

### Preparation of (S)—N-[2-Oxo-3-(1-oxo-1,2,4,5-tetrahydro-benzo[d]thiepin-7-yl)-oxazolidin-5-ylmethyl]-acetamide

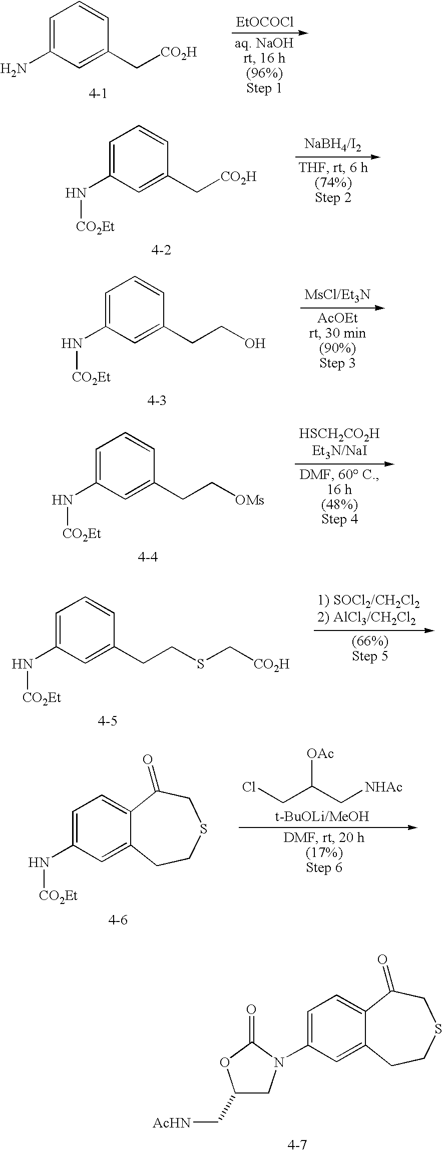

### (3-Ethoxycarbonylamino-phenyl)-acetic acid (Step 1)

To a solution of (3-Amino-phenyl)-acetic acid (10.6 g, 70.1 mmol) in 2 N NaOH (80 mL), cooled in an ice bath, was added ethyl chloroformate (8.37 g, 77.1 mmol) dropwise. The cooling bath was removed, and stirring was continued at room temperature for 16 hours. The mixture was then washed with diethyl ether, and the aqueous layer was separated, acidified with HCl, and the mixture was extracted with ethyl acetate. The ethyl acetate extracts were washed with brine and dried over Na₂SO₄ to give (3-ethoxycarbonylamino-phenyl)-acetic acid, which crystallized upon standing. Yield 15.1 g (96%). ¹H NMR (400 MHz, CDCl₃) δ 11.35 (br s, 1H), 7.28-7.20 (m, 3H), 6.96 (d, 1H), 6.68 (br s, 1H), 4.10 (q, 2H), 3.60 (s, 2H), 1.28 (t, 3H).

### [3-(2-Hydroxy-ethyl)-phenyl]-carbamic acid ethyl ester (Step 2)

To a stirring suspension of NaBH₄ (1.27 g, 33.6 mmol) in anhydrous tetrahydrofuran (30 mL), a solution of (3-ethoxycarbonylamino-phenyl)-acetic acid (5.0 g, 22.4 mmol) in anhydrous tetrahydrofuran (30 mL) was added at room temperature. Stirring was continued at room temperature for 5 minutes and a solution of I₂ (2.84 g, 11.2 mmol) in anhydrous tetrahydrofuran (30 mL) was added dropwise. The reaction mixture was stirred at room temperature for 6 hours, then it was cooled in an ice bath and quenched with 2 N HCl (20 mL). The mixture was extracted with ethyl acetate. The organic layer was separated, washed with water, 2 N NaOH, brine, dried over Na₂SO₄ and concentrated under vacuum. Purification by flash chromatography on silica gel (hexane/ethyl acetate 1:1) gave [3-(2-hydroxy-ethyl)-phenyl]-carbamic acid ethyl ester. Yield 3.49 g (74%).

¹H NMR (400 MHz, CDCl₃) δ 7.34-7.18 (m, 3H), 6.90 (d, 1H), 6.68 (br s, 1H), 4.22 (q, 2H), 3.83 (t, 2H), 2.82 (t, 2H), 1.62 (br s, 1H), 1.28 (t, 3H).

### Methanesulfonic acid 2-(3-ethoxycarbonylamino-phenyl)-ethyl ester (Step 3)

To a stirred solution of [3-(2-hydroxy-ethyl)-phenyl]-carbamic acid ethyl ester (10.5 g, 50 mmol) and triethylamine (6.10 g, 60 mmol) in dry ethyl acetate (100 mL), cooled to 0° C., was added a solution of methanesulfonyl chloride (6.30 g, 55 mmol) dropwise by syringe. Stirring was continued at 0° C. for 30 minutes, then the reaction mixture was diluted with ethyl acetate and washed with 2 N HCl, saturated aqueous NaHCO₃, brine, dried over Na₂SO₄ and concentrated under vacuum to give methanesulfonic acid 2-(3-ethoxycarbonylamino-phenyl)-ethyl ester as colorless solid. Yield 12.97 g (90%). ¹H NMR (400 MHz, CDCl₃) δ 7.38 (s, 1H), 7.28-7.18 (m, 2H), 6.93 (d, 1H), 6.66 (s, 1H), 4.20 (t, 2H), 4.12 (q, 2H), 3.02 (t, 2H), 2.97 (s, 3H), 1.31 (t, 3H).

### [2-(3-Ethoxycarbonylamino-phenyl)-ethylsulfanyl]-acetic acid (Step 4)

To a stirred solution of methanesulfonic acid 2-(3-ethoxycarbonylamino-phenyl)-ethyl ester (10.0 g, 34.8 mmol) in anhydrous N,N-dimethylformamide (80 mL), was added mercaptoacetic acid (3.53 g, 38.3 mmol), triethylamine (7.75 g, 76.6 mmol) and NaI (5.21 g, 34.8 mmol). The reaction mixture was stirred at 60° C. for 16 hours. N,N-Dimethylformamide was removed under vacuum, the residue was dissolved in ethyl acetate, washed with 2 N HCl and extracted with 5% NaOH. The basic aqueous extract was cooled, acidified with HCl and the mixture was extracted with dichloromethane. The dichloromethane extracts were washed with brine, dried over Na₂SO₄ and concentrated under vacuum to give acid [2-(3-ethoxycarbonylamino-phenyl)-ethylsulfanyl]-acetic acid as pale oil, which crystallized on standing. Yield 4.90 g (48%). ¹H NMR (400 MHz, CDCl₃) δ 10.38 (br s, 1H), 7.32-7.14 (m, 3H), 6.91 (d, 1H), 6.71 (br s, 1H), 4.12 (q, 2H), 3.14 (s, 2H), 2.92 (m, 2H), 1.31 (t, 3H).

### (1-Oxo-1,2,4,5-tetrahydro-benzo[d]thiepin-7-yl)-carbamic acid ethyl ester (Step 5)

To a stirred solution of acid [2-(3-ethoxycarbonylamino-phenyl)-ethylsulfanyl]-acetic acid (4.90 g, 17.2 mmol) in anhydrous dichloromethane (50 mL), was added thionyl chloride (4.1 g, 34.4 mmol) at room temperature, followed by N,N-dimethylformamide (5 drops). Stirring was continued at room temperature for 1.5 hours. Volatile components were removed under vacuum. The resulting residue was dissolved in anhydrous dichloromethane (20 mL) and then added dropwise to a vigorously stirred suspension of AlCl₃ (4.60 g, 34.4 mmol) in anhydrous dichloromethane (50 mL) which was cooled to 0° C. Stirring was continued at room temperature for 2.5 hours, then the reaction mixture was quenched with ice and extracted with dichloromethane. The organic extracts were washed with brine, dried over Na₂SO₄ and concentrated under vacuum. Purification by flash chromatography on silica gel (hexane/ethyl acetate, 3:1 to 1:1) gave (1-oxo-1,2,4,5-tetrahydro-benzo[d]thiepin-7-yl)-carbamic acid ethyl ester. Yield 3.02 g (66%). Melting Point 145-6° C. ESMS: m/z 266 (M+1). ¹H NMR (400 MHz, CDCl₃) δ 7.94 (d, 1H), 7.51 (s, 1H), 7.22 (dd, 1H), 6.88 (s, 1H), 4.14 (q, 2H), 3.54 (s, 2H), 3.28 (t, 2H), 2.98 (t, 2H), 1.33 (t, 3H).

### (S)—N-[2-Oxo-3-(1-oxo-1,2,4,5-tetrahydro-benzo[d]thiepin-7-yl)-oxazolidin-5-ylmethyl]-acetamide (Step 6)

To a stirred solution of (1-oxo-1,2,4,5-tetrahydro-benzo[d]thiepin-7-yl)-carbamic acid ethyl ester (1.50 g, 5.65 mmol) in anhydrous methanol (0.36 g, 11.3 mmol) and anhydrous N,N-dimethylformamide (6.0 mL), was added a solution of lithium tert-butoxide (17.0 mL of 1 M hexane solution, 17.0 mmol) dropwise over 1 h at room temperature. The mixture was cooled to 0° C. and N-(2-acetoxy-3-chloropropyl)acetamide (2.19 g, 11.5 mmol) was added as a solid in one portion. Stirring was continued at room temperature for 20 hours, then the mixture was quenched with saturated aqueous ammonium chloride (20 mL) and extracted with a large amount of ethyl acetate (approximately 350 mL). The combined organic extracts were washed with brine, dried over Na₂SO₄ and concentrated under vacuum. Purification by flash chromatography on silica gel (ethyl acetate/methanol, 10:1 to 3:1), followed by recrystallization from ethyl acetate/methanol gave the title compound. Yield 0.16 g (17%). Melting Point 232-3° C. ¹H NMR (400 MHz, DMSO-d₆) δ 8.24 (t, 1H), 7.83 (d, 1H), 7.63 (d, 1H), 7.48 (s, 1H), 4.75 (m, 1H), 4.16 (t, 1H), 3.77 (dd, 1H), 3.62 (s, 2H), 3.41 (t, 2H), 3.23 (t, 2H), 2.98 (t, 2H), 1.33 (t, 3H). ESMS: m/z 335 (M+1). C₁₆H₁₈N₂O₄S. Calcd, %: C, 57.47; H, 5.42; N, 8.38. Found, %: C, 57.41; H, 5.37; N, 8.25.

### EXAMPLE 5

### N-[2-Oxo-3-(1,3,3-trioxo-2,3,4,5-tetrahydro-1H-316-benzo[d]thiepin-7-yl)-oxazolidin-5-ylmethyl]-acetamide (5-1)

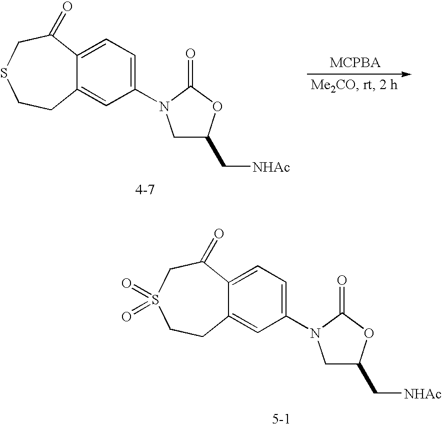

To a stirred suspension of N-[2-Oxo-3-(1-oxo-1,2,4,5-tetrahydro-benzo[d]thiepin-7-yl)-oxazolidin-5-ylmethyl]-acetamide (0.40 g, 1.2 mmol) in acetone (5.0 mL), cooled in an ice bath, was added meta-chloroperbenzoic acid (0.72 g of approximately 60% product, approximately2.5 mmol) was added as a solid. The cooling bath was removed and stirring was continued at room temperature for 2 hours. The mixture was purified directly by flash chromatography on silica gel (ethyl acetate-methanol, 10:1 to 6:1) to give the title compound as colorless crystals. Yield: 0.20 g (45%). Melting Point 225-6° C.

¹HNMR (400 MHz, DMSO-d₆) δ 8.24 (t, 1H), 7.78 (d, 1H), 7.68 (d, 1H), 7.53 (s, 1H), 4.88 (s, 2H), 4.76 (m, 1H), 4.12 (t, 1H), 3.77 (t, 1H), 3.56 (m, 4H), 3.41 (m, 2H), 1.81 (s, 3H). ESMS: m/z 367 (M+1). C₁₆H₁₈N₂O₆S. Calcd, %: C, 52.45; H, 4.95; N, 7.64. Found, %: C, 52.72; H, 5.16; N, 7.44.

### EXAMPLE 6

### (S)—N-[2-Oxo-3-(2-oxo-2,3,4,5-tetrahydro-1H-benzo[d][1,3]diazepin-7-yl)-oxazolidin-5-ylmethyl]-acetamide (6-9)

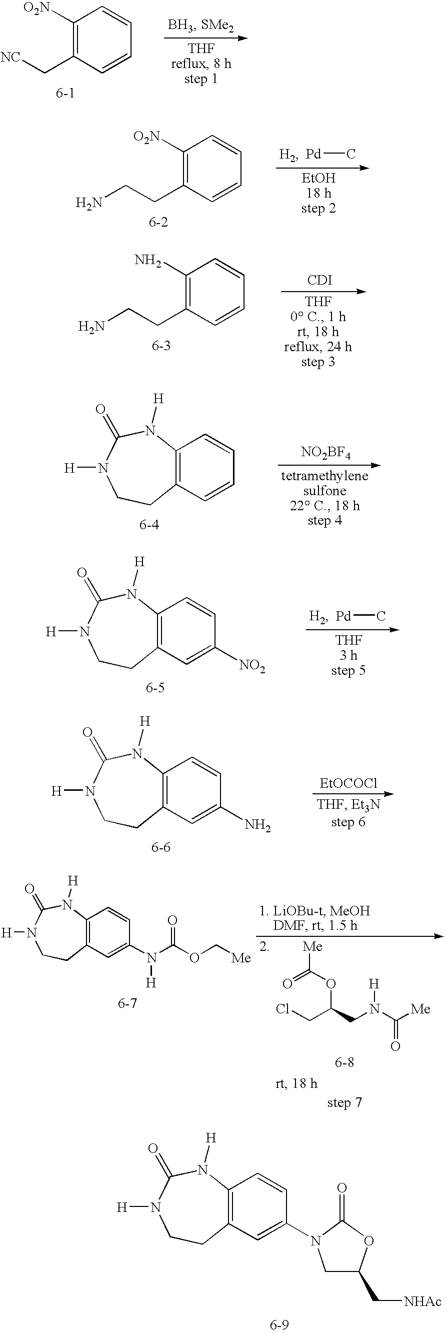

### 2-(2-Nitro-phenyl)-ethylamine (6-2) (Step 1)

To a solution of (2-nitro-phenyl)-acetonitrile (6.8 g, 42 mmol) in tetrahydrofuran (120 mL) was added borane-methylsulfide complex (96 mmol, 48 mL of 2M in tetrahydrofuran solution) at 0° C. After addition, the resulting solution was refluxed for 8 hours. After cooling to room temperature, the reaction mixture was quenched with 50 mL of methanol. The solvent was removed, and the residue was dissolved in 50 mL of methanol and refluxed for 1 h. The solution was concentrated and dried under vacuum to give 2-(2-nitro-phenyl)-ethylamine. (Yield: 7.0 g, quantitative). ¹H NMR (200 MHz, CDCl₃): δ 7.90 (d, 1H), 7.50 (t, 1H), 7.40 (m, 2H), 3.00 (s, 4H), 1.40 (br, 2H)

### 2-(2-Amino-ethyl)-phenylamine (6-3) (Step 2)

To a solution of 2-(2-nitro-phenyl)-ethylamine (7.0 g, 42 mmol) in ethanol (100 mL) was added 3.0 g of Pd—C (10%, wet). The mixture was hydrogenated under hydrogen at 40 psi for 18 h. After removal of the catalyst by filtration, the filtrate was concentrated under reduced pressure to give 2-(2-amino-ethyl)-phenylamine (Yield: 5.67 g, 99%). ¹H NMR (400 MHz, CDCl₃): δ 7.00 (m, 2H), 6.70 (m, 2H), 3.00 (t, 2H), 2.65 (t, 2H), 1.90 (br, 2H).

### 1,3,4,5-Tetrahydro-benzo[d][1,3]diazepin-2-one (6-4) (Step 3)

To a solution of 2-(2-amino-ethyl)-phenylamine (5.6 g, 41 mmol) in tetrahydrofuran (400 mL) was added 1,1′-carbonyldiimidazole (6.67 g, 41 mmol) at 0° C. with stirring. The solution was stirred at 0° C. for 1 h, then at room temperature for 18 h, and then refluxed for 24 h. The solid was removed by filtration. The filtrate was concentrated under vacuum, and the residue was purified by silica gel chromatography (2.5% to 5% of methanol in chloroform) to give the 1,3,4,5-tetrahydro-benzo[d][1,3]diazepin-2-one (Yield: 5.0 g, 75%). ¹H NMR (400 MHz, DMSO-d₆): δ8.60 (s, 1H), 7.05 (m, 4H), 6.80 (t, 1H), 3.20 (m, 2H), 2.85 (m, 2H). MS: m/z 163 (MH⁺).

### 7-Nitro-1,3,4,5-tetrahydro-benzo[d][1,3]diazepin-2-one (6-5) (Step 4)

To a suspension of 1,3,4,5-tetrahydro-benzo[d][1,3]diazepin-2-one (0.78 g, 4.8 mmol) in tetramethylenesulfone (15.5 g) was added nitronium tetrafluoroborate (90.76 g, 5.76 mmol) portion-wise over 0.5 hour at 22° C. After addition was complete, the mixture was stirred at 22° C. for 18 hours. The reaction mixture was diluted with 150 mL of water and stirred for 1 h. The solid was collected and dried to give 7-nitro-1,3,4,5-tetrahydro-benzo[d][1,3]diazepin-2-one (Yield: 0.47 g, 47%). ¹H NMR (400 MHz, DMSO-d₆): δ 9.40 (s, 1H), 8.00 (m, 2H), 7.45 (s, 1H), 7.20 (dd, 1H), 3.25 (m, 2H), 3.00 (m, 2H). MS: m/z 207.99 (MH⁺).

### 7-Amino-1,3,4,5-tetrahydro-benzo[d][1,3]diazepin-2-one and (2-Oxo-2,3,4,5-tetrahydro-1H-benzo[d][1,3]diazepin-7-yl)-carbamic acid ethyl ester (6-7) (Steps 5 and 6)

To a solution of 7-nitro-1,3,4,5-tetrahydro-benzo[d][1,3]diazepin-2-one (0.40 g, 1.93 mmol) in tetrahydrofuran (40 mL) was added 0.5 g of Pd—C (10%, wet). The mixture was hydrogenated under hydrogen at 45 psi for 3 hours. After removal of the catalyst by filtration, the filtrate (crude compound 6-6) was treated with 0.25 g (2.3 mmol) of ethyl chloroformate and 0.3 mL of triethylamine. The mixture was stirred at room temperature for 4 hours. The solid was removed by filtration and the filtrate was concentrated under vacuum. The residue was purified by chromatography using 5% to 10% of methanol in chloroform to give (2-Oxo-2,3,4,5-tetrahydro-1H-benzo[d][1,3]diazepin-7-yl)-carbamic acid ethyl ester 6-7 (Yield: 0.358 g, 75% in two steps). ¹H NMR (200 MHz, CDCl₃): δ 7.40 (s, 1H), 7.20 (s, 1H), 7.05 (d, 1H), 6.80 (d, 1H), 6.60 (s, 1H), 5.75 (s, 1H), 4.20 (q, 2H), 3.40 (m, 2H), 3.00 (m, 2H), 1.15 (t, 3H). MS: m/z 250.07 (MH⁺).

### (S)—N-[2-Oxo-3-(2-oxo-2,3,4,5-tetrahydro-1H-benzo[d][1,3]diazepin-7-yl)-oxazolidin-5-ylmethyl]-acetamide (Step 7)

To a solution of (2-Oxo-2,3,4,5-tetrahydro-1H-benzo[d][1,3]diazepin-7-yl)-carbamic acid ethyl ester 6-7 (0.34 g, 1.37 mmol) in a mixture of DMF (3 mL) and methanol (0.1 mL) was added lithium t-butoxide (4.1 mL, 4.1 mmol, 1M solution in hexanes) dropwise over 0.5 hour at 22° C. After addition was complete the mixture was stirred at 22° C. for 1 h. To this solution was added 0.318 g (1.64 mmol) of compound 6-8 in one portion at 22° C., and the resultant mixture was then stirred at 22° C. for 18 hours. The reaction mixture was quenched with 0.2 mL of 10 M hydrochloric acid. The resulting solution was directly purified by silica gel chromatography using 10% to 15% of methanol in chloroform to give 0.315 g of the target compound. A portion (90 mg) of it was further purified by crystallization from water to give the pure compound (62 mg). m.p 177-8° C. ¹H NMR (400 MHz, DMSO-d6): δ 8.60 (br, 1H), 8.28 (t, 1H), 7.25 (dd, 1H), 7.20 (d, 1H), 7.01 (d, 1H), 6.98 (br, 1H), 4.67 (m, 1H), 4.05 (t, 1H), 7.25 (dd, 1H), 3.38 (m, 2H), 3.20 (m, 2H), 2.90 (m, 2H), 1.80 (s, 3H). MS: m/z 319.08 (MH⁺).

### EXAMPLE 7

### N-[2-Oxo-3-(5-oxo-2,3,4,5-tetrahydro-benzo[b]oxepin-8-yl)-oxazolidin-5-ylmethyl]-acetamide (7-11)

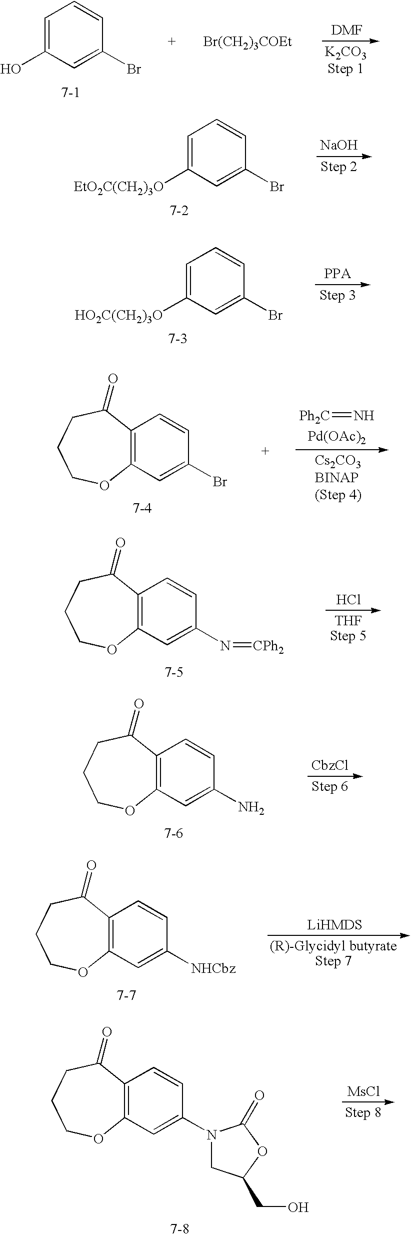

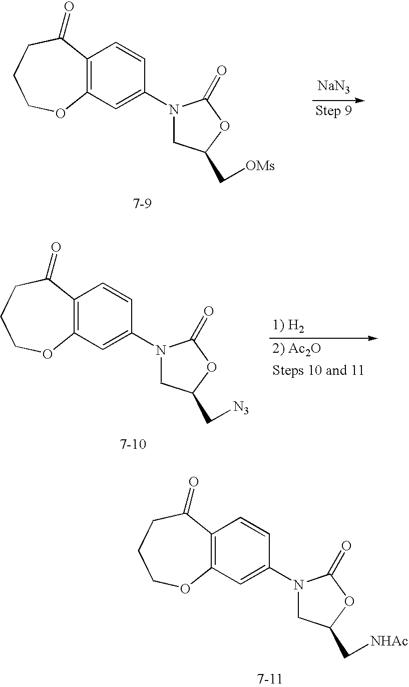

### 4-(3-Bromo-phenoxy)-butyric acid ethyl ester (7-2) (Step 1)

Potassium carbonate (319.0 g, 2.312 mol, 2 equiv) was added to a solution of 3-bromophenol (200 g, 1.156 mol, 1 equiv) and ethyl 4-bromobutryate (250 g, 1.282 mol, 1.11 equiv) in DMF (1.3 L). The reaction temperature increased from 28 to 33° C., then dropped back to room temperature. The suspension was stirred at room temperature for 39 h. The supernatant was decanted and the solids were slurried in heptane/MTBE (tert-butyl methyl ether) (1.5 L, 2:1 v/v) and filtered. The DMF solution was concentrated in vacuo to give an oil. The residual oil was partitioned between the heptane/MTBE washings and H₂O. The organic solution was washed sequentially with H₂O (2×0.5 L), 1 N NaOH (2×1 L), H₂O (4×0.5 20 L) and brine, then dried (Na₂SO₄), filtered and concentrated in vacuo. The resulting oil was placed under full vacuum for 1 h to give 336.7 g (102%) of ethyl 4-(3-bromophenoxy)butyrate [BR-1182-1], which was used in the next step without further purification.

### 4-(3-Bromo-phenoxy)-butyric acid (7-3) (Step 2)

A solution of 4-(3-bromo-phenoxy)-butyric acid ethyl ester (336.7 g, 1.117 mol, 1 equiv) in MeOH (1 L) was added to a solution of 85% KOH (116.3 g, 1.766 mol, 1.5 equiv) in H₂O (1 L), resulting in a temperature increase from 31 to 39° C. The cloudy reaction solution was stirred at room temperature over a weekend. The reaction solution was concentrated to ½ volume then cooled to approximately 10° C. and acidified to pH 2 with cold, dilute HCl to give an oil. Stirring and seeding induced crystallization. The solid was collected, washed with H₂O, pulverized and dried overnight in a vacuum oven at approximately 30-40° C. to give 290.6 g (96%) of 4-(3-bromophenoxy)butyric acid.

### 8-Bromo-3,4-dihydro-2H-benzo[b]oxepin-5-one (7-4) (Step 3)

A mixture of polyphosphoric acid (590 g), Celite (400 g) and toluene (1.5 L) were stirred for 0.5 h under N₂and then 4-(3-bromo-phenoxy)-butyric acid (135.8 g, 0.5264 mol) was added. The reaction mixture was refluxed for 2.5 h, and then cooled to room temperature. The reaction mixture was filtered and the Celite/PPA residue was washed with EtOAc (1.5 L). The organic solution was washed with 1 N NaOH (3×0.75 L), H₂O (1 L), brine, and then dried over Na₂SO₄. Concentration of the combined organic layers gave 94.3 g (75%) of 8-bromo-3,4-dihydro-2H-benzo[b]oxepin-5-one. This material was combined with 85.0 g of 8-bromo-3,4-dihydro-2H-benzo[b]oxepin-5-one from other runs (total weight—179.3 g) and distilled (oven temperature 130±5°, 0.18-0.20 mm) using a Kugelrohr apparatus to give 158.4 g of the title compound, mp 38.4-38.5° C.

### 8-(Benzhydrylidene-amino)-3,4-dihydro-2H-benzo[b]oxepin-5-one (7-5) (Step 4)

A mixture of Pd(OAc)₂ (4.03 g, 0.018 mol, 0.03 equiv), racemic BINAP [rac-2,2′-bis(diphenylphosphino)-1,1′-binaphthyl](14.9 g, 0.024 mol, 0.04 equiv) and toluene (2.25 L) was degassed with N₂ for 0.25 h. To this mixture was added 8-Bromo-3,4-dihydro-2H-benzo[b]oxepin-5-one (144.0 g, 0.6 mol, 1 equiv), Cs₂CO₃ (273 g, 0.84 mol, 1.4 equiv) and benzophenone imine (130.3 g, 120.7 mL, 0.72 mol, 1.2 equiv), and the reaction mixture was then heated to 90-95° C. for 17.5 h. The reaction mixture was cooled to room temperature and allow to stand over a weekend. The reaction mixture was filtered and the solids were washed with toluene (0.7 L). The filtrate was stripped to give 8-(benzhydrylideneamino)-3,4-dihydro-2H-benzo[b]oxepin-5-one, which was used in the next step without further purification.

### 8-Amino-3,4-dihydro-2H-benzo[b]oxepin-5-one (7-6) (Step 5)

A 2 M solution of HCl (150 mL) was added to a solution of crude 8-(benzhydrylidene-amino)-3,4-dihydro-2H-benzo[b]oxepin-5-one (from previous step) in THF (2 L) and the resultant reaction mixture was stirred overnight at room temperature. The mixture was concentrated in vacuo to give a residual oil which was partitioned between 2 M HCl (0.75 L) and heptane/EtOAc (1 L, 1:1 v/v). The aqueous solution was separated and washed with heptane/EtOAc (0.5 L, 1:1 v/v). The aqueous solution was made basic with cold aqueous NaOH to pH 10 and the aqueous mixture was then extracted with EtOAc (1×1 L, 1×0.5 L). The combined organic phases were washed with brine, dried (Na₂SO₄) and concentrated in vacuo to give 82.7 g (78% from step 4) of 8-amino-3,4-dihydrobenzo[b]oxepin-5-one [BR-1182-5A]. This material was used in the next step without purification, but the product from a 30 mmol front-run was purified by chromatography on silica gel, eluting with heptane, then heptane/EtOAc (2:1 v/v) to give the title compound.

### (5-Oxo-2,3,4,5-tetrahydro-benzo[b]oxepin-8-yl)-carbamic acid benzyl ester (7-7) (Step 6)

A mixture of 8-amino-3,4-dihydro-2H-benzo[b]oxepin-5-one (82.7 g, 0.4672 mol, 1 equiv) and solid NaHCO₃ (78.5 g, 0.9345 mol, 2 equiv) in THF (1.5 L) was cooled to approximately 7° C. under N₂. Benzyl chloroformate (92.0 g, 77 mL, 0.5140 mol, 1.1 equiv) was added dropwise over 0.5 h, maintaining the temperature at 6-7° C. After addition was complete, the resulting light suspension was stirred 1.5 h at 6-7° C., then allowed to warm to room temperature and stirred over a weekend. Analysis by TLC (heptane/EtOAc, 2:1) indicated that some starting material remained. Additional benzyl chloroformate (7.9 g, 6.6 mL, 0.1 equiv) was added and the reaction mixture was stirred overnight. The reaction mixture was filtered, and the solids were washed with TBF (0.5 L). The filtrate was concentrated in vacuo and the residual oil was dissolved in EtOAc (1.5 L). The organic solution was washed with saturated NaHCO₃ (2×0.5 L), H₂O (0.5 L), brine, dried (Na₂SO₄) and concentrated in vacuo to a volume of approximately 0.3 L. Heptane (0.5 L) was added and the oily mixture solidified. The solid was placed under vacuum to give a solid which was triturated with heptane/EtOAc (2:1) (2×1 L). After drying under full vacuum, 135.23 g (93%) of benzyl 5-oxo-2,3,4,5-tetrahydrobenzo[b]oxepin-8-yl carbamate was isolated.

### (R)-5-Hydroxymethyl-3-(5-oxo-2,3,4,5-tetrahydro-benzo[b]oxepin-8-yl)-oxazolidin-2-one (7-8) (Step 7)

A 1 M solution of LiHMDS in THF (320 mL, 0.3200 mol, 1.05 equiv) was cannulated into an addition funnel and added over 0.5 h to a solution of (5-oxo-2,3,4,5-tetrahydro-benzo[b]oxepin-8-yl)-carbamic acid benzyl ester (94.35 g, 0.3034 mol, 1 equiv) in TBF (1.5 L) cooled to −40° C. The temperature was maintained at −35 to −40° C. during the addition. After addition was complete, the suspension was stirred at −35 to −40° C. for 0.5 h. R-(−)-glycidyl butyrate (46.08 g, 45.2 mL, 0.3200 mol, 1.05 equiv) was added over 0.25 h to the reaction mixture at −35 to −40° C. After addition was complete, the reaction mixture was allowed to warm to room temperature and stirred overnight. The reaction mixture was quenched by the slow addition of saturated aqueous NH₄Cl (450 mL) and the organic phase was decanted. The aqueous phase was diluted with H₂O (500 mL) and extracted with EtOAc (1 L). The organic phases were combined, washed with brine, dried (Na₂SO₄) and concentrated in vacuo to give an oil. The crude reaction product was absorbed on to SiO₂ with dichloromethane, then added to a column of SiO₂ (1 kg) packed in heptane/EtOAc (3:1v/v). Elution with heptane/EtOAc (3:5) gave 16.45 g (16%) of 2-oxo-3-(5-oxo-2,3,4,5-tetrahydrobenzo[b]oxepin-8-yl)oxazolidin-5-ylmethyl butyrate as an oil that crystallized. Elution with heptane/EtOAc (1:4 v/v) to heptane/EtOAc (1:7 v/v) gave 56.10 g (67%) of 5-hydroxymethyl-3-(5-oxo-2,3,4,5-tetrahydrobenzo[b]oxepin-8-yl)oxazolidin-2-one [α]_{D}²²−49.6° (c=0.94, THF).

A mixture of 2-oxo-3-(5-oxo-2,3,4,5-tetrahydrobenzo[b]oxepin-8-yl)oxazolidin-5-ylmethyl butyrate (21.24 g, 0.0612 mol), KOH (4.8 g) and MeOH (350 mL) were stirred at room temperature for 1 h at which time TLC analysis (heptane/EtOAc, 1:3 v/v) indicated that saponification was complete. The suspension was concentrated in vacuo to a small volume, diluted with H₂O and the aqueous layer was extracted with EtOAc. The organic layer was washed with saturated NaHCO₃, brine, dried (Na₂SO₄) and concentrated in vacuo to approximately 300 mL. Heptane (100 mL) was added and the solution was seeded. Removal of the remaining solvent gave 11.94 g (70%) of 5-hydroxymethyl-3-(5-oxo-2,3,4,5-tetrahydro-benzo[b]oxepin-8-yl)-oxazolidin-2-one whose ¹H NMR spectrum was identical to that of the title compound obtained above.

### (R)-Methanesulfonic acid 2-oxo-3-(5-oxo-2,3,4,5-tetrahydro-benzo[b]oxepin-8-yl)-oxazolidin-5-ylmethyl ester (7-9) (Step 8)

A mixture of 5-hydroxymethyl-3-(5-oxo-2,3,4,5-tetrahydro-benzo[b]oxepin-8-yl)-oxazolidin-2-one (68.04 g, 0.2456 mol, 1 equiv), Et₃N (27.29 g, 37.6 mL, 0.2702 mol, 1.1 equiv) and THF (1.1 L) was cooled to 5° C. and then methanesulfonyl chloride (30.80 g, 20.8 mL, 0.2702 mol, 1.1 equiv) was added dropwise so as to maintain a temperature of 3-5° C. After addition was complete, the reaction suspension was stirred at 3-5° for 0.5 h and then allowed to warm to room temperature and stirred overnight. TLC analysis (heptane/EtOAc, 1:3) indicated that the reaction was not complete. Additional Et₃N (3.72 g, 5.1 mL, 0.0368 mol, 0.15 equiv) and methanesulfonyl chloride (4.2 g, 2.8 mL, 0.368 mol, 0.15 equiv) were added sequentially, and the reaction mixture stirred 4.5 h. The reaction mixture was filtered and the solids were washed with THF (0.6 L). The filtrate was concentrated to a small volume and diluted with EtOAc (1.25 L). The organic solution was washed with 1 N HCl (2×0.5 L), brine, dried (Na₂SO₄) and concentrated in vacuo to approximately 400 mL. The EtOAc solution was diluted with heptane (50 mL) and seeded. The remaining solvent was then removed under vacuum. The resulting solid was dried under full vacuum to give 85.29 g (98%) of methanesulfonic acid, 2-oxo-3-(5-oxo-2,3,4,5-tetrahydrobenzo[b]oxepin-8-yl)oxazolidin-5-ylmethyl ester, [α]_{D}²² 63.2° (c=0.97, THF).

### (R)-5-Azidomethyl-3-(5-oxo-2,3,4,5-tetrahydro-benzo[b]oxepin-8-yl)-oxazolidin-2-one (7-10) (Step 9)

A mixture of methanesulfonic acid 2-oxo-3-(5-oxo-2,3,4,5-tetrahydro-benzo[b]oxepin-8-yl)-oxazolidin-5-ylmethyl ester (85.29 g, 0.2403 mol, 1.0 equiv), NaN₃ (17.12 g, 0.2643 mol, 1.1 equiv) and DMF (0.7 L) was heated to 95-100° for 1.5 h, then allowed to cool slowly to room temperature and stirred overnight. The reaction mixture was poured into cold H₂O (2.5 L) and after several minutes a suspension formed. The solid was collected, washed with H₂O and then dried via filtration for approximately 1 h. The wet solid was dissolved in dichloromethane (2 L) and the organic solution washed with H₂O (1 L), brine, dried (Na₂SO₄) and concentrated in vacuo. The resulting solid was dried under full vacuum overnight to give 69.06 g (95%) of 5-azidomethyl-3-(5-oxo-2,3,4,5-tetrahydrobenzo[b]oxepin-8-yl)oxazolidin-2-one, [α]_{D}²²−144.2° (c=0.99, THF).

### (S)—N-[2-oxo-3-(5-oxo-2,3,4,5-tetrahydro-benzo[b]oxepin-8-yl)-oxazolidin-5-ylmethyl]-acetamide (7-11) (Steps 10 and 11)

A mixture of (R)-5-azidomethyl-3-(5-oxo-2,3,4,5-tetrahydro-benzo[b]oxepin-8-yl)-oxazolidin-2-one (68.88 g, 0.2282 mol, 1 equiv), 10% Pd—C (12.8 g, unreduced, 54% H₂O), acetic anhydride (100 mL, 1.06 mol, 4.6 equiv) and THF (0.9 L) was hydrogenated overnight at approximately 35 psi H₂. (Parr bottle was evacuated twice during the hydrogenation to remove N₂ being produced.) The reaction mixture was filtered through Celite, washing with THF (0.5 L) followed by MeOH (1 L), and the filtrate was stripped. The residual oil was dissolved in EtOAc (1 L) and saturated NaHCO₃ solution was added very slowly with stirring to neutralize HOAc. As neutralization occurred, a solid began forming at the interface. When all HOAc was neutralized, dichloromethane (approximately 2 L) was added to dissolve the solids. The organic solution was washed with brine, dried (Na₂SO₄) and the solvent stripped. A thick gel formed as the solution became concentrated. The crude reaction product, an amorphorous tan solid was redissolved in THF (approximately 0.75 L), absorbed on to SiO₂ and added to a column of SiO₂ (2.25 kg) packed in dichloromethane. The product eluted with dichloromethane/MeOH (97.5:2.5 v/v). Fractions containing the product were concentrated to approximately 500 mL and diluted with heptane (approximately 300 mL). Removal of the solvents gave initially a gel followed by a powder. Pumping overnight under high vacuum gave 47.20 g (65%) of (S)—N-[(2-oxo-3-(5-oxo-2,3,4,5-tetrahydrobenzo[b]oxepin-8-yl)oxazolidin-5-yl)methyl]acetamide, mp 123.4-127.8°, [α]D22-10.9° (c=1.006, THF).

### EXAMPLE 8

### N-[3-(3-Amino-2-oxo-2,3,4,5-tetrahydro-1H-benzo[b]azepin-7-yl)-2-oxo-oxazolidin-5-(S)-ylmethyl]-acetamide

### 1H,3H,4H,5H-benzo[f}azaperhydroepin-2-one (8-2) (Step 1)

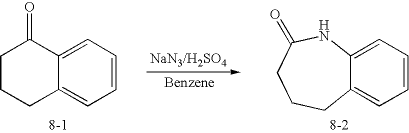

To a solution of α-tetralone (150 g, 1.02 moles) in toluene (2 L), sodium azide (267 g, 4.09 moles, ) was added at approximately 10° C., cooled to approximately 0° C. and _treated with concentrated sulfuric acid (160 mL, Caledon) with stirring, keeping the temperature below 5° C. The reaction mixture was stirred at room temperature _for another 16 hours, and the solvents were decanted. The solid residue was triturated in cold ether (1.5 L), filtered, washed with cold ether and dissolved in ethyl acetate-methanol mixture (1:1, 2×1.5 L). The mixture was concentrated to a thick paste, which was precipitated with ether and dried under vacuum at approximately 55° C. to afford the title compound. Yield: 40g (24%), m.p.134-37° C.

### 7-Nitro-1H,3H,4H,5H-benzo[f]azaperhydroepin-2-one (8-3) (Step 2)

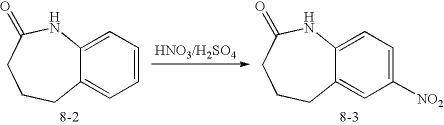

To a cold (approximately 5° C.) stirred solution of concentrated sulfuric acid (120 mL) and 90% nitric acid (20 mL) was added 1H,3H,4H,5H-benzo[f}azaperhydroepin-2-one, 2 (65 g, 0.403 moles, 02-036-060 & 066) in portions over 1 H at 0-5° C. The mixture was stirred for another 1 H at approximately 5° C. The dark thick solution was added to crushed ice while stirring and the resulting mixture was stirred for 30 minutes. The solids were filtered, washed with water to neutral pH, and dried under vacuum at 50° C. to give the title compound. Yield: 56 g, m.p.205-08° C. (decomp.).

### 7-Amino-1,3,4,5-tetrahydro-benzo[b]azepin-2-one (8-4) (Step 3)

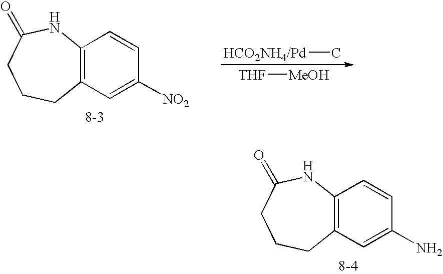

To a solution of 7-nitro-1,3,4,5-tetrahydro-benzo[b]azepin-2-one (0.20 g, 0.97 mmol) in a mixture of tetrahydrofuran and methanol (8 mL/2 mL), ammonium formate (0.245 g, 3.88 mmol) was added, followed by 10% palladium on carbon. The mixture was stirred at room temperature for 2 hours and filtered through celite. The filtrate was concentrated under vacuum, treated with water and extracted with ethyl acetate. The organic extracts were washed with brine and dried over Na₂SO₄ to give the title compound. Yield 0.13 g (76%). ¹H NMR (400 MHz, CDCl₃) δ 7.38 (br s, 1H), 6.80 (d, 1H), 6.56 (m, 2H), 3.62 (br s, 2H), 2.70 (t, 2H), 2.38 (m, 2H), 2.20 (t, 2H).

### (R)-Butyric acid 2-hydroxy-3-(2-oxo-2,3,4,5-tetrahydro-1H-benzo[b]azepin-7-ylamino)-propyl ester (8-5) (Step 4)

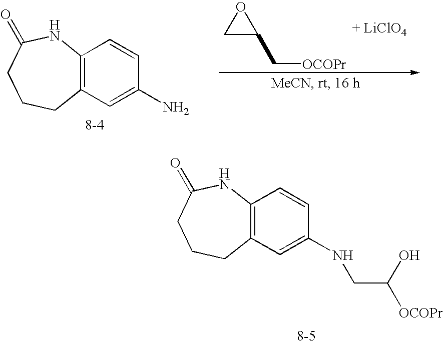

To a stirred solution of 7-amino-1,3,4,5-tetrahydro-benzo[b]azepin-2-one (0.90 g, 5.1 mmol) and (R)-glycidyl butyrate (0.74 g, 5.1 mmol) in anhydrous acetonitrile (45 mL), lithium perchlorate (0.70 g, 6.6 mmol) was added, and the mixture was stirred at room temperature for several hours. The solvent was removed under vacuum and the residue was partitioned between water and ethyl acetate. The organic layer was separated, washed with water, saturated NaHCO₃ and brine, dried over Na₂SO₄ and concentrated under vacuum. Purification by column chromatography on silica gel (ethyl acetate/methanol 19:1) gave (R)-Butyric acid 2-hydroxy-3-(2-oxo-2,3,4,5-tetrahydro-1H-benzo[b]azepin-7-ylamino)-propyl ester. Yield 1.2 g (75%). ¹H NMR (400 MHz, CDCl₃) δ 8.24 (s, 1H), 6.78 (d, 1H), 6.48 (m, 2H), 4.30 (br s, 1H), 4.20 (m, 2H), 4.10 (m, 1H), 3.92 (br s, 1H), 3.28 (m, 1H), 3.12 (m, 1H), 2.63 (t, 2H), 2.31 (t, 2H), 2.28 (m, 2H), 2.11 (m, 2H), 1.67 (m, 2H), 0.92 (t, 3H). MS: m/z 320 (M⁺).

### (R)-Butyric acid 2-oxo-3-(2-oxo-2,3,4,5-tetrahydro-1H-benzo[b]azepin-7-yl)-oxazolidin-5-ylmethyl ester (8-6) (Step 5)

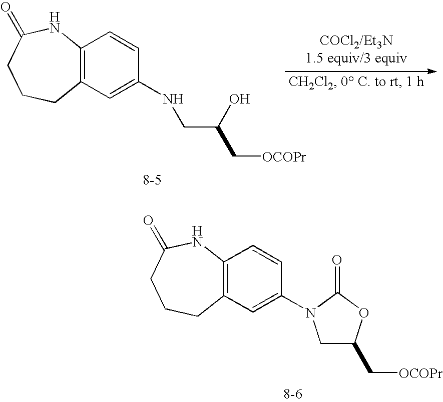

To a mixture of (R)-butyric acid 2-hydroxy-3-(2-oxo-2,3,4,5-tetrahydro-1H-benzo[b]azepin-7-ylamino)-propyl ester (0.48 g, 1.50 mmol) and triethylamine (0.46 g, 4.50 mmol) in anhydrous dichloromethane (15 mL), cooled to 0° C., was added a solution of phosgene (1.16 mL of 1M toluene solution, 2.20 mmol) dropwise by syringe. Stirring was continued at 0° C. for 30 minutes, then at room temperature for 1 hour. Saturated aqueous NaHCO₃ was then added. The organic layer was separated and extracted with dichloromethane. The combined extracts were washed with brine, dried over Na₂SO₄ and concentrated under vacuum. Purification by flash chromatography on silica gel (ethyl acetate/methanol, 100:1 to 40:1) gave (R)-butyric acid 2-oxo-3-(2-oxo-2,3,4,5-tetrahydro-1H-benzo[b]azepin-7-yl)-oxazolidin-5-ylmethyl ester. Yield 0.15 g (29%). ¹H NMR (400 MHz, CDCl₃) δ 8.32 (s, 1H), 7.48 (m, 1H), 7.28 (m, 1H), 6.98 (d, 1H), 4.87 (m, 1H), 4.42-4.26 (m, 2H), 4.11 (t, 1H), 3.82 (dd, 1H), 2.78 (t, 2H), 2.31 (m, 4H), 2.22 (t, 2H), 1.62 (m, 2H), 0.91 (t, 3H). MS: m/z 347 (M⁺+1).

### (R)-Butyric acid 3-(3-iodo-2-oxo-2,3,4,5-tetrahydro-1H-benzo[b]azepin-7-yl)-2-oxo-oxazolidin-5-ylmethyl ester (8-7) (Step 6)

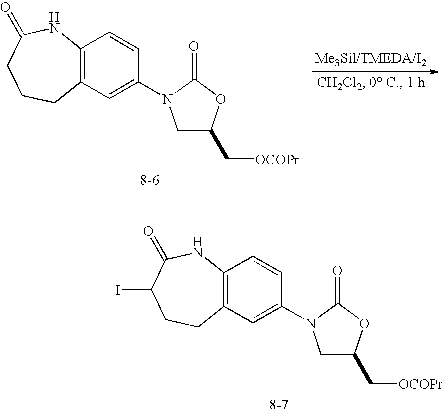

To a solution of (R)-butyric acid 2-oxo-3-(2-oxo-2,3,4,5-tetrahydro-1H-benzo[b]azepin-7-yl)-oxazolidin-5-ylmethyl ester (0.75 g, 2.15 mmol) in anhydrous dichloromethane (6.0 mL), cooled to 0° C., was added N,N,N′N′-tetramethylenediamine (0.75 g, 6.48 mmol), followed by 1-iodotrimethylsilane (1.30 g, 6.48 mmol). The mixture was stirred at 0° C. for 30 minutes Solid iodine (0.82 g, 3.24 mmol) was added in one portion and stirring was continued at 0° C. for 1 hour. The mixture was diluted with dichloromethane and treated with 10% Na₂SO₃ solution until the color of iodine disappeared. The aqueous layer was separated and extracted with dichloromethane, and then the combined organic extracts were washed with brine, dried over Na₂SO₄ and concentrated under vacuum. Purification by flash chromatography on silica gel (hexane/ethyl acetate, 1:3 to 1:2) gave the title compound. Yield 0.79 g (77%). ^{1H}NMR (400 MHz, CDCl₃) δ 7.78 (s, 1H), 7.56 (m, 1H), 7.33 (m, 1H), 7.02 (d, 1H), 4.98 (m, 1H), 4.66 (t, 1H), 4.44-4.26 (m, 2H), 4.14 (t, 1H), 3.83 (m, 1H), 2.98 (m, 1H), 2.85-2.65 (m, 3H), 2.33 (t, 2H), 1.63 (m, 2H), 0.91 (t, 3H). MS: m/z 472 (M⁺).

### (R)-Butyric acid 3-(3-azido-2-oxo-2,3,4,5-tetrahydro-1H-benzo[b]azepin-7-yl)-2-oxo-oxazolidin-5-ylmethyl ester (8-7) (Step 7)

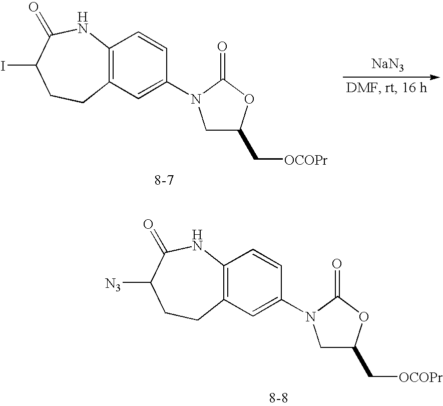

A mixture of 8-7 (0.77 g, 1.63 mmol) and NaN₃ (1.06 g, 16.3 mmol) in anhydrous N,N-dimethylformamide (10 mL), was stirred at room temperature for 16 hours. The mixture was partitioned between water and ethyl acetate, and the organic layer was separated, washed with water and brine, dried over Na₂SO₄ and concentrated under vacuum. Purification by flash chromatography on silica gel (hexane/ethyl acetate, 1:3 to 1:2) gave the title compound. Yield 0.49 g (79%). ¹H NMR (400 MHz, CDCl₃) δ 7.76 (s, 1H), 7.58 (s, 1H), 7.31 (m, 1H), 7.03 (d, 1H), 4.88 (m, 1H), 4.47-4.28 (m, 2H), 4.12 (m, 1H), 3.85 (m, 2H), 2.98 (m, 1H), 2.77 (m, 1H), 2.51 (m, 1H), 2.39-2.24 (m, 3H), 1.62 (m, 2H), 0.92 (t, 3H). IR (Nujol) 2100 cm⁻¹. MS: m/z 320 (M⁺+1).

### (R)-Butyric acid 3-(3-amino-2-oxo-2,3,4,5-tetrahydro-1H-benzo[b]azepin-7-yl)-2-oxo-oxazolidin-5-ylmethyl ester (8-9) (Step 8)

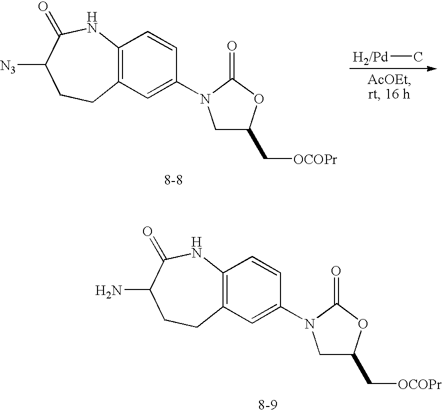

A mixture of (R)-butyric acid 3-(3-azido-2-oxo-2,3,4,5-tetrahydro-1H-benzo[b]azepin-7-yl)-2-oxo-oxazolidin-5-ylmethyl ester. (0.49 g, 1.26 mmol) and 10% palladium on carbon (0.29 g) in ethyl acetate (20 mL) was stirred under H₂ (atmospheric pressure) at room temperature for 16 hours. The mixture was filtered through celite and the filtrate was concentrated under vacuum to give the title compound. Yield 0.44 g (98%). ¹H NMR (400 MHz, CDCl₃) δ 7.67 (s, 1H), 7.49 (m, 1H), 7.35 (m, 1H), 6.98 (d, 1H), 4.88 (m, 1H), 4.42-4.28 (m, 2H), 4.12 (m, 1H), 3.83 (m, 1H), 3.41 (m, 1H), 2.93 (m, 1H), 2.68 (m, 1H), 2.51 (m, 1H), 2.32 (t, 2H), 1.83 (m, 1H), 1.78 (br s, 2H), 1.64 (m, 2H), 0.92 (t, 3H). MS: m/z 362 (M⁺+1).

### (R)-Butyric acid 3-(3-benzyloxycarbonylamino-2-oxo-2,3,4,5-tetrahydro-1H-benzo[b]azepin-7-yl)-2-oxo-oxazolidin-5-ylmethyl ester (8-10) (Step 9)

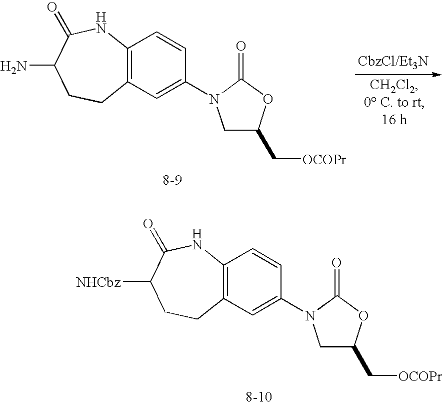

To a stirred solution of (R)-butyric acid 3-(3-amino-2-oxo-2,3,4,5-tetrahydro-1H-benzo[b]azepin-7-yl)-2-oxo-oxazolidin-5-ylmethyl ester (0.22 g, 0.61 mmol) and triethylamine (0.12 g, 1.22 mmol) in anhydrous dichloromethane (3.0 mL), benzyl chloroformate (0.16 g of 85% product, 0.79 mmol) was added dropwise at 0° C. Stirring was continued at 0° C. for 1 hour and at room temperature for 16 hours. The mixture was diluted with ethyl acetate, washed with saturated NaHCO₃ solution and brine, dried over Na₂SO₄ and concentrated under vacuum. Purification by flash chromatography on silica gel (hexane/ethyl acetate, 1:2 to 1:3) gave the title compound. Yield 0.15 g (50%). ^{1H}NMR (400 MHz, CDCl₃) δ 8.10 (d, 1H), 7.49 (d, 1H), 7.38-7.21 (m, 6H), 6.98 (d, 1H), 5.79 (d, 1H), 5.04 (s, 2H), 4.88 (m, 1H), 4.44-4.24 (m, 2H), 4.11 (t, 1H), 3.83 (m, 1H), 2.93 (m, 1H), 2.76-2.62 (m, 2H), 2.34 (t, 2H), 2.01 (m, 1H), 1.64 (m, 2H), 0.92 (t, 3H). MS: m/z 496 (M⁺+1).

### (R)-[7-(5-Hydroxymethyl-2-oxo-oxazolidin-3-yl)-2-oxo-2,3,4,5-tetrahydro-1H-benzo[b]azepin-3-yl]-carbamic acid benzyl ester (8-11) (Step 10)

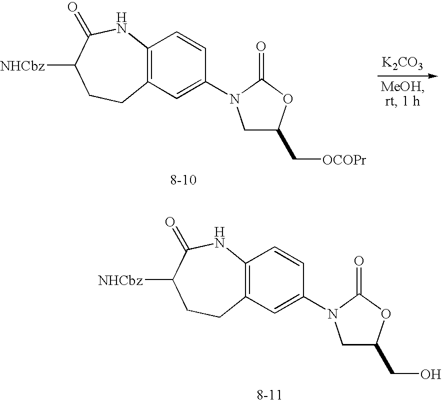

To a stirred solution of butyric acid 3-(3-benzyloxycarbonylamino-2-oxo-2,3,4,5-tetrahydro-1H-benzo[b]azepin-7-yl)-2-oxo-oxazolidin-5-ylmethyl ester (0.15 g, 0.29 mmol) in methanol (6.0 mL), solid K₂CO₃ (0.20 g, 1.45 mmol) was added at room temperature and the resulting suspension was stirred at room temperature for 1 hour. The mixture was concentrated under vacuum to one quarter of its volume and the residue was partitioned between ethyl acetate and water. The organic layer was separated, washed with water and brine, dried over Na₂SO₄ and concentrated under vacuum to give the title compound. Yield 0.13 g (quantitative). ¹H NMR (400 MHz, CDCl₃) δ 7.60 (d, 1H), 7.50 (d, 1H), 7.41-7.22 (m, 6H), 6.97 (m, 1H), 5.74 (d, 1H), 5.05 (s, 2H), 4.76 (m, 1H), 4.29 (m, 1H), 4.08-3.97 (m, 2H), 3.77 (m, 1H), 2.92 (m, 1H), 2.74-2.60 (m, 2H), 2.37 (m, 1H), 1.99 (m, 1H). MS: m/z 425 (M⁺+1).

### (R)-Methanesulfonic acid 3-(3-benzyloxycarbonylamino-2-oxo-2.3,4,5-tetrahydro-1H-benzo[b]azepin-7-yl)-2-oxo-oxazolidin-5-ylmethyl ester (8-12) (Step 11)

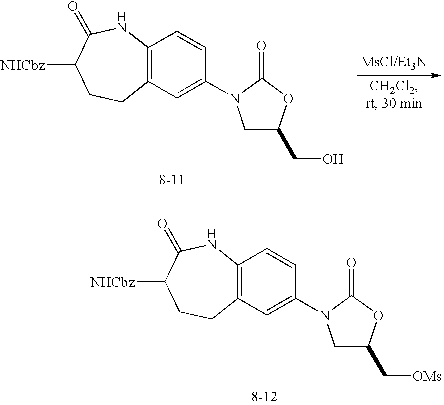

To a stirred solution of (R)-[7-(5-hydroxymethyl-2-oxo-oxazolidin-3-yl)-2-oxo-2,3,4,5-tetrahydro-1H-benzo[b]azepin-3-yl]-carbamic acid benzyl ester (0.12 g, 0.29 mmol) and triethylamine (0.06 g, 0.58 mmol) in anhydrous dichloromethane (5.0 mL), a solution of methanesulfonyl chloride (0.04 g, 0.35 mmol) in anhydrous dichloromethane (0.5 mL) was added dropwise at room temperature and stirring was continued at room temperature for 30 minutes. The mixture was diluted with ethyl acetate and washed with water, 2 N HCl, saturated NaHCO₃ solution and brine; the solution was dried over Na₂SO₄ and concentrated under vacuum to give the title compound. Yield 0.15 g (quantitative). ¹H NMR (400 MHz, CDCl₃) δ 7.56-7.44 (m, 2H), 7.42-7.27 (m, 6H), 7.00 (d, 1H), 5.71 (m, 1H), 5.05 (s, 2H), 4.95 (m, 1H), 4.64-4.42 (m, 2H), 4.30 (m, 1H), 4.16 (m, 1H), 3.98 (m, 1H), 3.12 (s, 3H), 2.94 (m, 1H), 2.77-2.63 (m, 2H), 2.02 (m, 1H). MS: m/z 503 (M⁺+1).

### (R)-[7-(5-Azidomethyl-2-oxo-oxazolidin-3-yl)-2-oxo-2,3,4,5-tetrahydro-1H-benzo[b]azepin-3-yl]-carbamic acid benzyl ester (8-13) (Step 12)

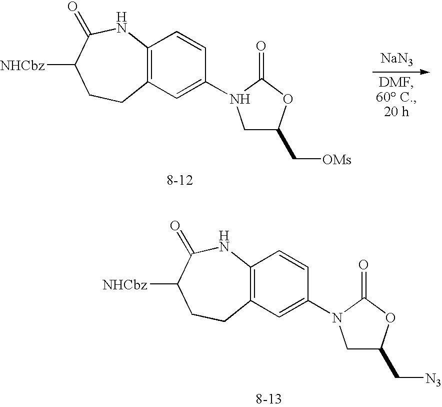

A mixture of (R)-methanesulfonic acid 3-(3-benzyloxycarbonylamino-2-oxo-2,3,4,5-tetrahydro-1H-benzo[b]azepin-7-yl)-2-oxo-oxazolidin-5-ylmethyl ester (0.15 g. 0.29 mmol) and NaN₃ (0.19 g, 2.9 mmol) in anhydrous N,N-dimethylformamide (3.0 mL) was stirred at 60° C. for 20 hours. The reaction mixture was diluted with ethyl acetate, washed with water and brine, dried over Na₂SO₄ and concentrated under vacuum to give the title compound. Yield 0.12 g (95%). ¹H NMR (400 MHz, CDCl₃) δ 7.51 (d, 1H), 7.42-7.22 (m, 7H), 7.01 (d, 1H), 5.71 (d, 1H), 5.07 (s, 2H), 4.81 (m, 1H), 4.31 (m, 1H), 4.11 (m, 1H), 3.88 (m, 1H), 3.73 (m, 1H), 3.62 (m, 1H), 2.97 (m, 1H), 2.75-2.63 (m, 2H), 2.01 (m, 1H). IR (Nujol) 2100 cm⁻¹. MS: m/z 451 (M⁺+1).

### (S)-{7-[5-(Acetylamino-methyl)-2-oxo-oxazolidin-3-yl]-2-oxo-2,3,4,5-tetrahydro-1H-benzo[b]azepin-3-yl}-carbamic acid benzyl ester (8-14) (Step 13)

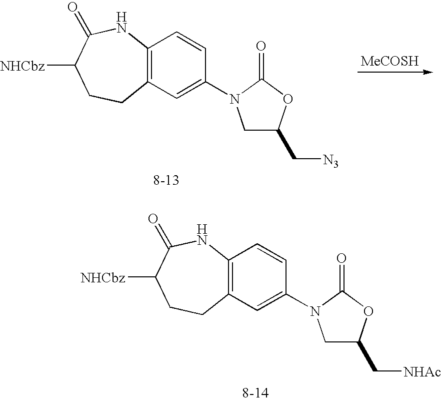

A solution of [7-(5-azidomethyl-2-oxo-oxazolidin-3-yl)-2-oxo-2,3,4,5-tetrahydro-1H-benzo[b]azepin-3-yl]-carbamic acid benzyl ester (120 mg, 0.27 mmol) in thioacetic acid (3.0 mL) was stirred at room temperature for 16 hours. Thioacetic acid was removed under vacuum and the residue was purified by flash chromatography on silica gel (neat ethyl acetate, then ethyl acetate/methanol, 10:1 to 5:1) to give the title compound. Yield 84 mg (67%). ¹H NMR (400 MHz, CDCl₃) δ 7.54 (d, 1H), 7.49-7.36 (m, 1H), 7.34-7.27 (m, 6H), 6.97 (d, 1H), 6.11 (t, 1H), 5.71 (m, 1H), 5.04 (s, 2H), 4.79 (m, 1H), 4.29 (m, 1H), 4.11-4.02 (m, 1H), 3.78 (m, 1H), 3.69 (m, 2H), 2.94 (m, 1H), 2.68 (m, 2H), 2.05 (s, 3H), 2.04-1.92 (m, 1H). MS: m/z 466 (M⁺+1).

### (S)—N-[3-(3-Amino-2-oxo-2,3,4,5-tetrahydro-1H-benzo[b]azepin-7-yl)-2-oxo-oxazolidin-5-ylmethyl]-acetamide (8-15) (Step 14)

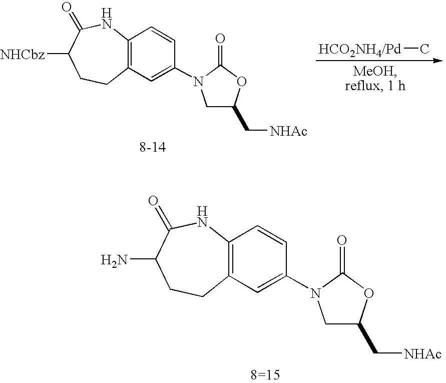

A mixture of (S)-{7-[5-(acetylamino-methyl)-2-oxo-oxazolidin-3-yl]-2-oxo-2,3,4,5-tetrahydro-1H-benzo[b]azepin-3-yl}-carbamic acid benzyl ester (80 mg, 0.17 mmol), ammonium formate (107 mg, 1.7 mmol) and 10% palladium on carbon (50 mg) in anhydrous methanol (10 mL) was stirred under reflux for 1 hour. The mixture was filtered through celite and concentrated under vacuum. Purification by preparative thin-layer chromatography on silica gel (dichloromethane/methanol/triethylamine, 150:13:5) gave the title compound. Melting Point>160° C. (decomp.) Yield 49 mg (87%). ¹H NMR (400 MHz, DMSO-D₆) δ 9.72 (s, 1H), 8.25 (t, 1H), 7.47-7.36 (m, 2H), 6.97 (d, 1H), 4.68 (m, 1H), 4.09 (m, 1H), 3.71 (m, 1H), 3.50 (br. s, 2H), 3.38 (m, 2H), 3.17 (m, 1H), 2.69-2.56 (m, 2H), 2.28 (m, 1H), 1.81 (s, 3H), 1.80-1.71 (m, 1H). MS: m/z 332 (M⁺). C₁₆H₂₀N₄O₄.1/2H₂O Calcd, %: C, 56.30; H, 6.20; N, 16.41. Found, %: C, 56.00; H, 6.58; N, 15.19.

### EXAMPLE 9

### (S)—N-[(3-(1H,2H,3H,4H,5H-benzo[3,4-f]azaperhydroepin-7-yl)-2-oxo-1,3-oxazolidin-5-yl)methyl]acetamide

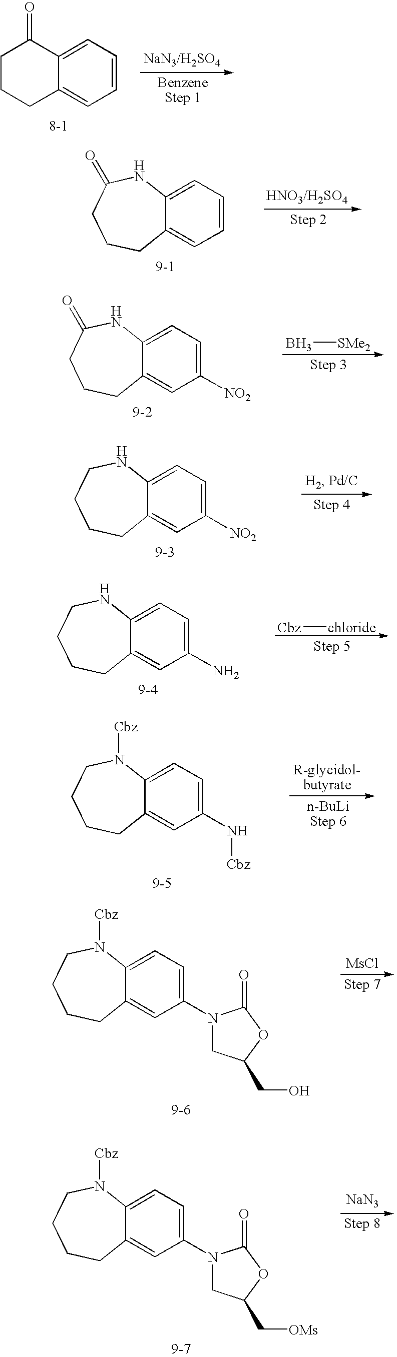

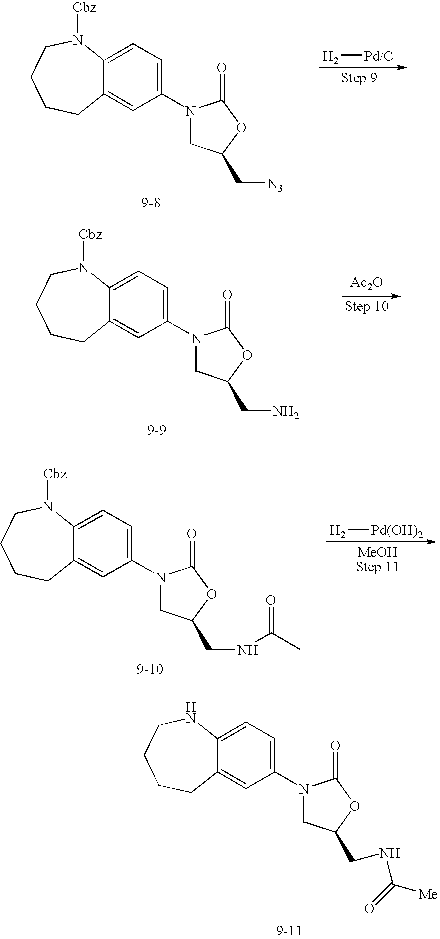

### 1H,3H,4H,5H-benzo[f}azaperhydroepin-2-one (9-1) (Step 1)

To a solution of α-tetralone 8-1 (100 g, 0.685 moles) in toluene (1.4 L,), sodium azide (178 g, 2.73 moles) was added at approximately 10° C. and then cooled to approximately 0° C. Sulfuric acid (160 mL,) was added with stirring, over 6 hours, keeping the temperature below 5° C. The reaction mixture was stirred at room temperature for another 2 days and the solvents were decanted. The solid residue was triturated in cold ether (1 L), filtered, washed with cold ether and dissolved in ethyl acetate-methanol mixture (1:1, 2×1 L); the solution was concentrated to a thick paste, which was precipitated with ether and dried under vacuum at approximately 55° C. to afford the title compound. Yield: 55 g (98%), m.p.135-37° C.

### 7-Nitro-1H,3H,4H,5H-benzo[f]azaperhydroepin-2-one (9-3) (Step 2)

To a cold (approximately 5° C.) stirred solution mixture of concentrated sulfuric acid (120 mL) and 90% nitric acid (20 mL) was added 1H,3H,4H,5H-benzo[f]azaperhydroepin-2-one (50 g, 0.310 moles, 02-036-080) in portions during 1H and the mixture was stirred for another 1 hour at approximately 5° C. The thick solution was added to crushed ice while stirring and was stirred for 30 minutes. The solids were filtered, washed with water to neutral pH and dried under vacuum at 50° C. to give the title compound. Yield: 40 g, m.p.204-207° C., (decomp.).

### 7-Nitro-1H,2H,3H,4H,5H-benzo[f]azaperhydroepin-2-one (9-4) (Step 3)

To a solution of 7-nitro-1H,3H,4H,5H-benzo[f]azaperhydroepin-2-one (55 g, 0.267 mole) in dry TBF (250 mL) was added borane-methyl sulfide complex (75 mL, 0.667 moles, 10M solution in THF) dropwise at room temperature over 15 minutes The reaction mixture was then stirred at 40° C. for 16 hours and then carefully quenched with 150 mL while cooling (approximately 10° C.). The reaction mixture was saturated with bubbling HCl gas and the solvents were removed under vacuum. The residue was triturated in ether, filtered, washed with ether and dried under vacuum at approximately 50° C. to give the title compound. Yield: 58 g (Quantitative)

### 1H,2H,3H,4H,5H-benzo[f]azaperhydroepine-7-ylamine (9-4) (Step 4)

To a solution of 7-nitro-1H,2H,3H,4H,5H-benzo[f]azaperhydroepin-2-one, 4 (58 g, 0.301 moles) in methanol (650 mL) was added 6.6 g of 10% Pd/C under nitrogen atmosphere and the mixture was hydrogenated at 50 psi and room temperature during 16 hours. The reaction mixture was filtered, washed with methanol, concentrated and vacuum dried at approximately50° C. to afford the title compound Yield: 53 g (Quantitative), m.p.195-98° C.

### Phenylmethyl-7-[(phenylmethoxy)carbonylamino]-2H,3H,4H,5H-benzo[f]azaperhydroepinecarboxylate (9-5) (Step 5)

To a solution of 1H,2H,3H,4H,5H-benzo[f]azaperhydroepine-7-ylamine (53 g, 0.326 moles) in dichloromethane (500 mL) was added diisopropylethyl amine (173.1 g, 234 mL, 1.34 moles). The resulting mixture was stirred for 15 minutes and then benzylchloroformate (111.86 g, 94 mL, 0.655 moles) was added dropwise over 30 minutes at 10-15° C. The reaction mixture was stirred at room temperature for 3 hours, diluted with dichloromethane (400 mL), water (150 mL) and 2N HCl (150 mL), and stirred, and the layers were separated. The organic phase was washed with water (2×200 mL), brine (250 mL), dried over sodium sulfate, adsorbed on silica gel, and chromatographed over silica gel (20% -30% ethyl acetate in hexane) to afford the title compound. Yield: 63 g (44%)

### Phenylmethyl-7-[5-(hydroxymethyl)-2-oxo-1,3-oxazolidin-3-yl]-2H,3H,4H,5H-benzo[f]azaperhydroepinecarboxylate (9-6) (Step 6)

To a solution of phenylmethyl-7-[(phenylmethoxy)carbonylamino]-2H,3H,4H,5H-benzo[f]azaperhydroepinecarboxylate (66 g, 0.153 moles) in dry THF (700 mL, Aldrich) was added n-BuLi (75 mL, 2.5 M solution in hexanes, 0.176 moles) at −70 to —75° C. over 1 hour. The resulting mixture was stirred for 1 hour and then treated with (R)-glycidyl butyrate (25.7 g, 0.168 moles) at −70° C. The reaction mixture was brought to room temperature over 2 hours, warmed to approximately 35° C. and stirred for 2 hours. The reaction mixture was then diluted with ethyl acetate (500 mL) followed by water (375 mL) and stirred for 15 minutes. The organic phase was separated, washed with brine (250 mL), dried over sodium sulfate, adsorbed on silica gel, and chromatographed over silica gel (33%-50% ethyl acetate in hexanes) to give the title compound. Yield: 41.5 g (68%), m.p.103-05° C.

### Phenylmethyl-7-{5-[(methoxysulfinyloxy)methyl]-2-oxo-1,3-oxazolidin-3-yl}-2H,3H,4H,5H-benzo[f]azaperhydroepinecarboxylate (9-7) (Step 7)

To a solution of phenylmethyl-7-[5-(hydroxymethyl)-2-oxo-1,3-oxazolidin-3-yl]-2H,3H,4H,5H-benzo[f]azaperhydroepinecarboxylate, 9-6 (41.5 g, 0.105 moles) in dichloromethane (450 mL) was added diisopropylethylamine, (DIPEA, 27 g, 0.209 moles). The resulting mixture was stirred for 15 minutes, and then methane sulfonyl chloride (18 g, 0.157 moles) was added dropwise over 30 minutes at 10-15° C. The reaction mixture was brought to room temperature and stirred for 16 hours. The reaction solution was diluted with dichloromethane (400 mL), water (100 mL), and 2N HCl (100 mL), and stirred,; the layers were separated. The organic phase was washed with water (2×200 ML), brine (250 mL), dried over sodium sulfate, and evaporated to dryness to give the title compound solid. Yield: 49 g (Quantitative), m.p. 139-42° C.

### Phenylmethyl-7-[5-(azidomethyl)-2-oxo-1,3-oxazolidin-3-yl]-2H,3H,4H,5H-benzo[f]azaperhydroepinecarboxylate (9-8) (Step 8)

To a solution of phenylmethyl-7-{5-[(methoxysulfinyloxy)methyl]-2-oxo-1,3-oxazolidin-3-yl}-2H,3H,4H,5H-benzo[f]azaperhydroepinecarboxylate, 9-7 (49 g, 0.103 moles) in DMF (900 mL) was added sodium azide (13.4 g, 0.206 moles) and heated at 80° C. with stirring for 1.5 hours. The reaction solution was then diluted with ethyl acetate (1 L) and water (750 mL), stirred, and the layers were separated. The organic phase was washed with water (2×200 mL) and brine (250 mL), dried over sodium sulfate, and evaporated to dryness to give the title compound which was used in the next reaction without purification. Yield: 45 g (Quantitative)

### Phenylmethyl-7-[5-(aminomethyl)-2-oxo-1,3-oxazolidin-3-yl]-2H,3H,4H,5H-benzo[f]azaperhydroepinecarboxylate (9-9) (Step 9)

To a solution of phenylmethyl-7-[5-(azidomethyl)-2-oxo-1,3-oxazolidin-3-yl]-2H,3H,4H,5H-benzo[f]azaperhydroepinecarboxylate (45 g, 0.106 moles) in ethyl acetate (600 mL) was added 7.2 g of 10% Pd/C under nitrogen atmosphere and hydrogenated at atmospheric pressure and room temperature during 60 hours. The reaction mixture was filtered, washed with methanol, concentrated and vacuum dried at approximately 50° C. to afford the title compound Yield: 41 g (97%).

### Phenylmethyl-7-{5-[(acetylamino)methyl]-2-oxo-1,3-oxazolidin-3-yl)-2H,3H 4H,5H-benzo[f]azaperhydroepinecarboxylate (9-10) (Step 10)

To a solution of phenylmethyl-7-[5-(aminomethyl)-2-oxo-1,3-oxazolidin-3-yl]-2H,3H,4H,5H-benzo[f]azaperhydroepinecarboxylate, 9-9 (41 g, 0.1036 moles) in dichloromethane (400 mL) was added DIPEA (20.1 g, 0.155 moles,). The solution was stirred for 15 minutes and was treated with acetic anhydride (11.63 g, 0.114 moles) drop wise during 10 minutes at 10-15° C. The reaction mixture was brought to room temperature, stirred for 3 hours and diluted with dichloromethane (400 mL), water (100 mL) and 2N HCl (100 mL), The layers were separated. The organic phase was washed with water (2×200 mL), brine (250 mL), dried over sodium sulfate, and evaporated to dryness to give the title compound foam. Yield: 42 g (92.7).

### N-[(3-(1H,2H,3H,4H,5H-benzo[3,4-f]azaperhydroepin-7-yl)-2-oxo-1,3-oxazolidin-5-yl)methyl]acetamide (9-11) (Step 11)

To a solution of phenylmethyl-7-{5-[(acetylamino)methyl]-2-oxo-1,3-oxazolidin-3-yl }-2H,3H,4H,5H-benzo[f]azaperhydroepinecarboxylate (41.5 g, 0.0948 moles) in methanol (600 mL) was added 17 g of 20% Pd(OH)2/C under nitrogen atmosphere. The resulting mixture was hydrogenated at atmospheric pressure and room temperature for 16 hours. The catalyst was filtere and washed with methanol; the filtrate was concentrated and vacuum dried at approximately 50° C. The residue was purified by chromatography over silica gel, using 2% methanol in chloroform to afford the title compound. Yield: 20 g (70%). m.p. 104-107° C.

### EXAMPLE 10

### (S)—N-[2-oxo-3-(5-oxo-1,3,4,5-tetrahydro-benzo[c]thiepin-8-yl)-oxazolidin-5-ylmethyl]-acetamide

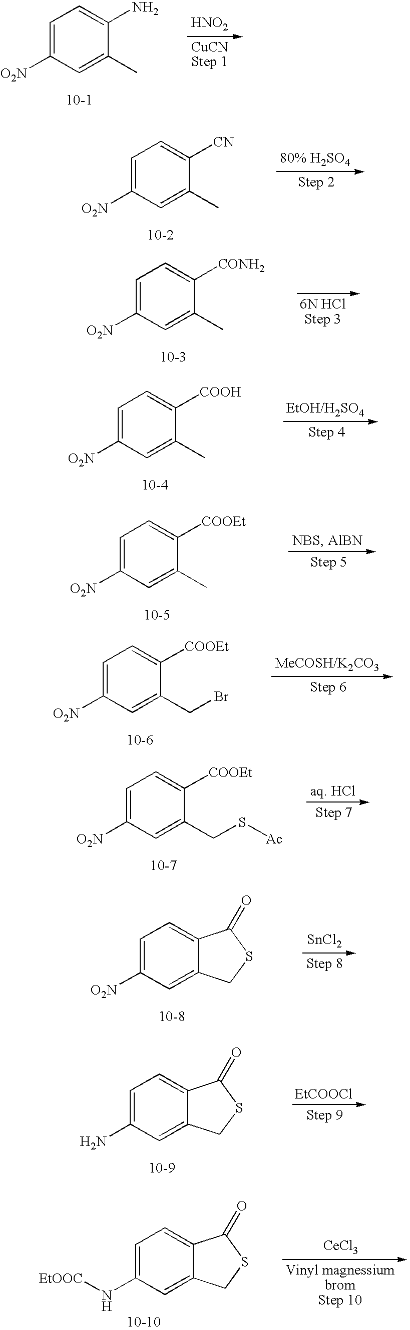

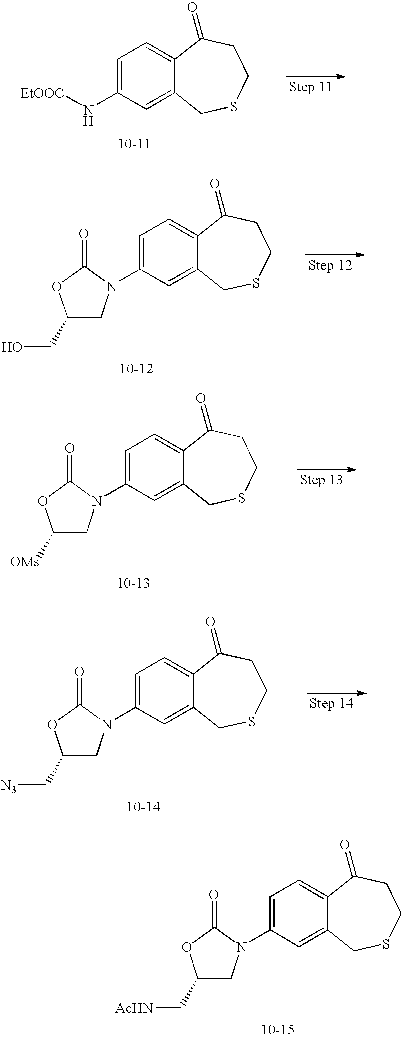

### 2-Methyl-4-nitrobenzenecarbonitrile (10-2) (Step 1)

To a suspension of 2-methyl4-nitroaniline (91.0 g, 0.6 mol) in 12 N HCl (150 mL) was added crushed ice (300 g) and the mixture was stirred at −5° C., while adding a solution of NaNO₂ (42.88 g, 0.62 mol) in water (120 mL) in portions. After neutralization at 0-5° C. with aqueous NaHCO₃, the mixture was added drop-wise with cooling and stirring to a two phase mixture of ethyl acetate (600 mL) and a solution of CuCN (53.70 g, 0.6 mol) and KCN (90 g, 1.38 mol) in water (300 mL). After the completion of addition the mixture was allowed to warm up to room temperature and left to stand overnight. The reaction mixture was filtered and the organic layer was separated and the aqueous layer was extracted with ethyl acetate (3×100 mL). The combined ethyl acetate extracts were evaporated and the residue was stirred in warm hexane along with sufficient amount of acetone to dissolve almost all the solid. The warm solution was filtered and the filtrate was cooled to give the title compound. Yield: 90 g (92.80%).

### 2-Methyl-4-nitrobenzamide (10-3) (Step 2)

A solution of the 2-methyl-4-nitrobenzenecarbonitrile (160.0 g, 0.98 mol) in 80% sulfuric acid (700 mL) was heated in an oil bath at 100° C. for 2 hours, cooled to room temperature and poured onto crushed ice. The resulting solid was filtered and used without purification in the next step.

### 2-Methyl-4-nitrobenzoic acid (10-4) (Step 3)

A stirred mixture of 2-methyl-4-nitrobenzamide and 6N HCl (1.3 L) was refluxed for 20 hours, then cooled. The resulting solid was filtered, washed with cold water, and dried on a lyophilizer to obtain the title compound as a solid. Yield: 160 g (Overall yield of last two steps was 89.50%), Melting Point: 149-150° C.

### Ethyl-2-methyl-4-nitrobenzoate (10-5) (Step 4)

To a solution of 2-methyl4-nitrobenzoic acid 120.0 g, 0.662 mol) in ethanol (1.0 L) was added concentrated sulfuric acid (1.0 mL) and the mixture was heated at reflux for 24 hours and evaporated. The residue was dissolved in ethyl acetate (1.0 L), washed with saturated aqueous NaHCO₃ solution (2×100 mL) and brine (1×200 mL), dried over anhydrous sodium sulfate, filtered and evaporated under vacuum to give the title compound, which was used in the next step without further purification. Yield: 115 g (82.99%), Melting Point: 71-72° C.

### Ethyl-2-(bromomethyl)-4-nitrobenzoate (10-6) (Step 5)

To a solution of ethyl-2-methyl4nitrobenzoate (68.0 g, 0325 mol) in CCl₄ (1.0 L) was added N-bromosuccinimide (63.7 g, 0357 mol) and AIBN (8.0 g, 0.048 mol). The reaction mixture was heated to reflux for 48 hours under nitrogen and light, then cooled to room temperature and filtered. The filtrate was concentrated and the residue was dissolved in ethyl acetate (100 mL), washed with brine (2×100 mL) and concentrated. The residue was purified by silica gel chromatography (5% EtOAc:Hex) to give the title compound. Yield: 56.0 g (59.97%), Melting Point: 65-66° C.

### Ethyl-2-(acetylthiomethyl)-4-nitrobenzoate (10-7) (Step 6)

Ethyl-2-(bromomethyl)-4-nitrobenzoate (56.0 g, 0.195 mol), potassium carbonate (31.11 g, 0.224 mol) and dry acetone (2.0 L) at 0° C. were charged to a flask and the mixture was added drop wise to thiolacetic acid (24.21 g, 22.81 mL, 0.319 mol). After stirring at 0° C. for 30 minutes, the cooling bath was removed and the reaction mixture was allowed to warm to room temperature then stirred for 3 hours under nitrogen. The solid formed was filtered and the filtrate was concentrated under vacuum. The residue was purified by column chromatography (EtOAc:Hex 10:90) to give the title compound. Yield: 50 g (90.56%),

### 5-Nitro-3-hydrobenzo[c]thiophen-1-one (10-8) (Step 7)

To the neat ethyl-2-(acetylthiomethyl)-4-nitrobenzoate, 7 (100 g, 0.353 mol) was added concentrated aqueous HCl (1.0 L) and the mixture was heated at reflux for 2 hours, then cooled. The solid that separated was filtered, washed with water (500 mL) and dried in a vacuum oven to give the title compound. Yield: 60.0 (87.08%).

### 5-Amino-3-hydrobenzo[c]thiophen-1-one (10-9) (Step 8)

To a solution of 5-nitro-3-hydrobenzo[c]thiophen-1-one (58.0 g, 0.297 mol) in ethyl acetate (1.5 L) was added SnCl₂.2H₂O (345.0 g, 1.52 mol) and the reaction mixture was heated at reflux for 2 hours under nitrogen. After cooling to room temperature the mixture was adjusted to pH 8 with saturated aqueous NaHCO₃ solution, and then water was added. The aqueous layer was extracted with ethyl acetate (3×500 mL) and the combined organic layers were washed with brine, dried over anhydrous Na₂SO₄, filtered and evaporated under vacuum to give the title compound 9. Yield: 39.0 g (79.47%)

### Ethoxy-N-{1-oxo(3-hydrobenzo[c]thiophen-5-yl)}carboxamide (10-10) (Step 9)

To a mixture of 5-amino-3-hydrobenzo[c]thiophen-1-one (22.5 g, 0.136 mol) and pyridine (44 mL) in dry dichloromethane (1.5 L) was added ethyl chloroformate (29.51 g,26.0 mL, 0.271 mol) dropwise at 0° C. After stirring at this temperature for 0.5 hour, the cooling bath was removed and the reaction mixture was stirred at room temperature for 5 hours, and then treated with water. The organic layer was separated and the aqueous layer was extracted with dichloromethane (3×500 mL). The combined organic layers were washed with brine (2×200 mL), dried over anhydrous Na₂SO₄, filtered and concentrated under vacuum to give the title compound 10. Yield: 16.0 g (49.52%), Melting Point: 150-151° C.

### (5-Oxo-1,3,4,5-tetrahydro-benzo[c]thiepin-8-yl)-carbamic acid ethyl ester (10-11) (Step 10)

To a solution of ethoxy-N-{1-oxo(3-hydrobenzo[c]thiophen-5-yl)}carboxamide (0.64 g, 2.7 mmol) in THF (25 mL) at −78° C. were added CeCl₃ (0.133 g, 0.54 mmol) and vinyl magnesium bromide (1.0 M solution in THF, 6.75 mL). The mixture was stirred at this temperature for 2 hours, and at room temperature overnight. The mixture was then treated with glacial acetic acid (0.52 mL) and 5 mL water. The layers were separated and the water layer was extracted with ether. The combined ether layers were washed with saturated sodium bicarbonate, dried over sodium sulfate, filtered, and evaporated. The crude product was purified by chromatography (silica gel, 30% ethyl aceate in hexane) to give the desired product 0.23 g (32%). ¹H NMR (400 MHz, CDCl₃) ppm: 7.70 (dd, 1H), 7.42 (d, 1H), 7.26 (dd, 1H), 6.72 (bs, 1H), 4.25 (a, 2H), 3.84 (s, 2H), 3.10 (m, 2H), 2.82 (m, 2H), 1.36 (t, 3H).

### (R)-5-Hydroxymethyl-3-(5-oxo-1,3,4,5-tetrahydro-benzo[c]thiepin-8-yl)-oxazolidin-2-one (10-12) (Step 11)

To a solution of starting (5-oxo-1,3,4,5-tetrahydro-benzo[c]thiepin-8-yl)-carbamic acid ethyl ester (0.3 g, 1.13 mmol) in THF (15 mL) at −78° C. was added dropwise n-butyllithium (2.5 M, 0.48 mL, 1.2 mmol) and the reaction mixture was stirred at this temperature for 1 hour. Glycidyl butyrate (0.17 mL, 1.2 mmol) in 0.3 mL THF was then added and the mixture was stirred at this temperature for 1 hour and room temperature overnight. Saturated aqueous ammonium chloride was added, and the mixture was extracted with ethyl acetate. The organic layers were dried with sodium sulfate and evaporated. The crude product was purified by chromatography (50% ethyl acetate in hexane) to give the title compound (yield: 40%). ¹H NMR (400 MHz, CDCl₃) δ: 7.78 (dd, 1H), 7.62 (d, 1H), 7.42 (dd, 1 H), 4.80 (m, 1H), 4.10 (m, 3H), 3.90 (s, 2H), 3.80 (m, 1H), 3.12 (m, 2H), 2.82 (m, 2H), 1.96 (t, 1H).

### (R)-Methanesulfonic acid 2-oxo-3-(5-oxo-1,3,4,5-tetrahydro-benzo[c]thiepin-8-yl)-oxazolidin-5-ylmethyl ester (10-13) (Step 12)

To a solution of (R)-5-hydroxymethyl-3-(5-oxo-1,3,4,5-tetrahydro-benzo[c]thiepin-8-yl)-oxazolidin-2-one (0.13 g, 0.44 mmol) and triethyl amine (0.12 mL, 0.88 mmol) in dichloromethane (10 mL) at 0° C. was added methane sulfonyl chloride (0.05 mL, 0.66 mmol). The reaction mixture was allowed to stir at room temperature for 4 hours, and then was partitioned between saturated sodium bicarbonate solution and dichloromethane. The organic layers were collected and washed with brine and dried with sodium sulfate to give a crude oil (0.187 g), which was directly used in the next step. ¹H NMR (400 MHz, CDCl₃) δ: 7.78 (dd, 1H), 7.62 (d, 1H), 7.42 (dd, 1H), 4.95 (m, 1H), 4.50 (m, 2H), 4.22 (t, 1H), 4.05 (m, 1H), 3.90 (s, 2H), 3.15 (m, 2H), 3.12 (s, 3H), 2.85 (m, 2H).

### (R)-5-Azidomethyl-3-(5-oxo-1,3,4,5-tetrahydro-benzo[c]thiepin-8-yl)-oxazolidin-2-one (10-14) (Step 13)

To a solution of (R)-methanesulfonic acid 2-oxo-3-(5-oxo-1,3,4,5-tetrahydro-benzo[c]thiepin-8-yl)-oxazolidin-5-ylmethyl ester in DMF (5 mL) was added sodium azide (0.14 g, 2.2 mmol). After being heated at 65° C. overnight, the reaction mixture was cooled, diluted with ethyl acetate and washed with water and brine to give an oily product (0.122 g), which was used directly in the next step. ¹H NMR (400 MHz, CDCl₃) δ: 7.80 (dd, 1H), 7.62 (d, 1H), 7.42 (dd, 1H), 4.85 (m, 1H), 4.10 (m, 1H), 3.90 (m, 1H), 3.88 (s, 2H), 3.70 (m, 2H), 3.15 (m, 2H), 2.85 (m, 2H).

### (S)—N-[2-Oxo-3-(5-oxo-1,3,4,5-tetrahydro-benzo[c]thiepin-8-yl)-oxazolidin-5-ylmethyl]-acetamide (10-15) (Step 14)

A mixture of starting (R)-5-azidomethyl-3-(5-oxo-1,3,4,5-tetrahydro-benzo[c]thiepin-8-yl)-oxazolidin-2-one and thiol acetic acid (4 mL) was stirred at room temperature for 4 hours and concentrated. The residue was subjected to flash chromatography (4% methanol in dichloromethane) to give the desired product (0.067 g, 45% over three steps). ¹H NMR (400 MHz, CDCl₃) δ: 7.72 (dd, 1H), 7.55 (dd, 1H), 7.45 (dd, 1H), 6.20 (t, 1H), 4.82 (m, 1H), 4.10 (t, 1H), 3.90 (s, 2H), 3.85 (m, 1H), 3.70 (m, 2H), 3.15 (m, 2H), 2.85 (m, 2H). Mass Spec m/z (ES): 334.88.

### EXAMPLE 11

### (S)—N-[3-(2-Dimethylaminomethylene-1-oxo-1,2,4,5-tetrahydro-benzo[d]thiepin-7-yl)-2-oxo-oxazolidin-5-ylmethyl]-acetamide 11-1

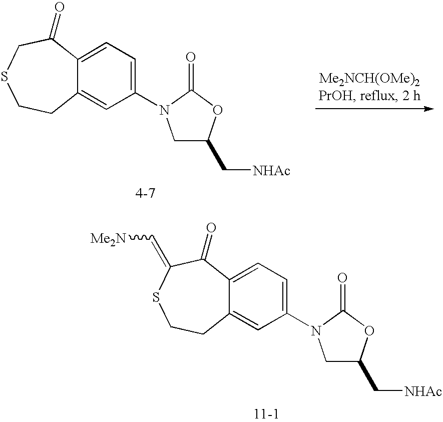

To a stirred solution of (S)—N-[2-oxo-3-(1-oxo-1,2,4,5-tetrahydro-benzo[d]thiepin-7-yl)-oxazolidin-5-ylmethyl]-acetamide (0.45 g, 1.3 mmol) in n-propanol (15 mL), dimethylformamide dimethylacetal (0.62 g, 5.2 mmol) was added dropwise at room temperature. Stirring was continued under reflux for 2 hours. After cooling to the room temperature, a precipitate appeared, which was filtered away, washed with n-propanol and dried to provide the first portion of the title compound (0.20 g). The mother liquor was concentrated and purified by flash chromatography on silica gel (ethyl acetate-methanol, 10:1 to 5:1) to give additionally 0.09 g of the tile compound. Combined yield: 0.29 g (57%). Melting Point 221-2° C. ¹H NMR (400 MHz, CDCl₃) δ: 8.12 (s, 1H), 7.68 (d, 1H), 7.51 (d, 1H), 7.33 (dd, 1H), 5.96 (t, 1H), 4.77 (m, 1H), 4.08 (t, 1H), 3.79 (dd, 1H), 3.77-3.67 (m, 1H), 3.63-3.53 (m, 1H), 3.25 (br s, 6H), 2.94 (t, 2H), 2.83 (br s, 2H), 2.02 (s, 3H). ESMS: m/z 390 (M+1).

### EXAMPLE 12

### (S)—N-[3-(5,6-Dihydro-1H-4-thia-1,2-diaza-benzo[e]azulen-8-yl)-2-oxo-oxazolidin-5-ylmethyl]-acetamide 12-1

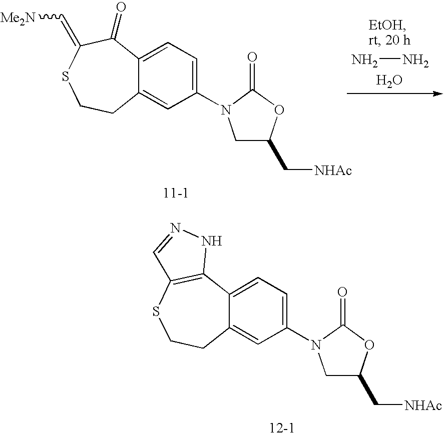

To a stirred suspension of (S)—N-[3-(2-dimethylaminomethylene-1-oxo-1,2,4,5-tetrahydro-benzo[d]thiepin-7-yl)-2-oxo-oxazolidin-5-ylmethyl]-acetamide (0.20 g, 0.51 mmol) in ethanol (6.0 mL), was added hydrazine hydrate (0.20 g, 4.0 mmol) and stirring was continued at room temperature for 20 hours. The mixture was purified directly by flash chromatography on silica gel (ethyl acetate-methanol, 10:1 to 5:1) give the title compound. Yield: 0.17 g (94%). Melting Point 186-187° C. ¹H NMR (400 MHz, DMSO-d₆) δ: 13.33 and 13.03 (s each, 1H), 8.28 (t, 1H), 7.87 and 7.60 (s each, 1H), 7.46 (m, 3H), 4.72 (m, 1H), 4.12 (t, 1H), 3.77 (t, 1H), 3.41 (m, 2H), 3.28 (m, 2H), 2.82 (m, 2H), 1.82 (s, 3H). ESMS: m/z 359 (M+1). C₁₇H₁₈N₄O₃S. Calcd, %: C, 56.97; H, 5.06; N, 15.63. Found, %: C, 56.59; H, 4.80; N, 15.13.

### EXAMPLE 13

### (S)—N-[3-(2-Amino-6,7-dihydro-5-thia-1,3-diaza-dibenzo[a,c]cyclohepten-9-yl)-2-oxo-oxazolidin-5-ylmethyl]-acetamide 13-1

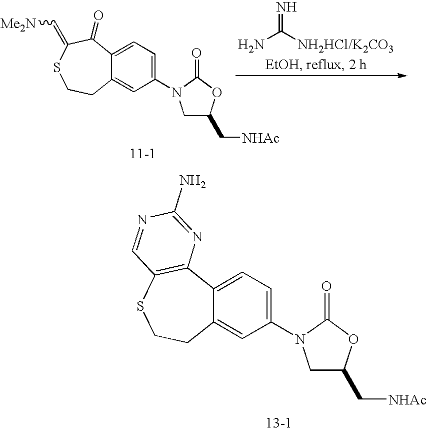

To a stirred suspension of (S)—N-[3-(2-dimethylaminomethylene-1-oxo-1,2,4,5-tetrahydro-benzo[d]thiepin-7-yl)-2-oxo-oxazolidin-5-ylmethyl]-acetamide (111 mg, 0.28 mmol) in ethanol (6.0 mL), was added guanidine hydrochloride (267 mg 2.8 mmol), followed by K₂CO₃ (387 mg, 2.8 mmol), and stirring was continued under reflux for 2 hours. After cooling to the room temperature, the reaction mixture solidified. A small amount of water (0.5-1.0 mL) was added and the mixture was purified directly by flash chromatography on silica gel (ethyl acetate-methanol, 10:1 to 4:1) to give the title compound. Yield: 85 mg (79%). Melting Point 190-1° C. (dec). ¹H NMR (400 MHz, DMSO-d₆) δ 8.27 (t, 1H), 8.22 (s, 1H), 7.64 (dd, 1H), 7.58 (d, 1H), 7.50 (s, 1H), 6.96 (s, 2H), 4.75 (m, 1H), 4.17 (t, 1H), 3.79 (m, 1H), 3.43 (t, 2H), 3.28 (t, 2H), 2.77 (t, 2H), 1.82 (s, 3H). ESMS: m/z 386 (M+1). C₁₈H₁₉N₅O₃S. Calcd, %: C, 56.09; H, 4.97; N, 18.17. Found, %: C, 55.32; H, 5.20; N, 17.13.

### EXAMPLE 14

### (S)—N-[3-(2-Dimethylaminomethylene-1,3,3-trioxo-2,3,4,5-tetrahydro-1H-316-benzo[d]thiepin-7-yl)-2-oxo-oxazolidin-5-ylmethyl]-acetamnide 14-1

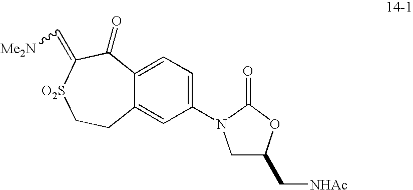

To a stirred solution of N-[2-oxo-3-(1,3,3-trioxo-2,3,4,5-tetrahydro-1H-316-benzo[d]thiepin-7-yl)-oxazolidin-5-ylmethyl]-acetamide 11-1 (0.52 g, 1.4 mmol) in n-propanol (20 mL), dimethylformamide dimethylacetal (0.67 g, 5.6 mmol) was added dropwise at room temperature and stirring was continued under reflux for 2 hours. After cooling to the room temperature, a precipitate appeared, which was filtered away, washed with n-propanol and dried to give the title compound Yield 0.56 g (95%). Melting Point 261-3° C. ¹H NMR (400 MHz, CDCl₃): δ 7.84 (d, 1H), 7.79 (s, 1H), 7.61 (d, 1H), 7.35 (dd, 1H), 6.18 (t, 1H), 4.75 (m, 1H), 4.07 (t, 1H), 3.79 (m, 1H), 3.71-3.52 (m, 2H), 3.47 (m, 2H), 3.37 (s, 3H), 3.30 (t, 2H), 3.02 (s, 3H), 2.02 (s, 3H). ESMS: m/z 422 (M+1).

### EXAMPLE 15

### (S)—N-[3-(4,4-Dioxo-1,4,5,6-tetrahydro-416-thia-1,2-diaza-benzo[e]azulen-8-yl)-2-oxo-oxazolidin-5-ylmethyl]-acetamide 15-1

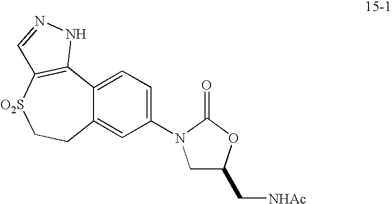

To a stirred suspension of (S)—N-[3-(2-Dimethylaminomethylene-1,3,3-trioxo-2,3,4,5-tetrahydro-1H-316-benzo[d]thiepin-7-yl)-2-oxo-oxazolidin-5-ylmethyl]-acetamide 14-1 (200 mg, 0.47 mmol) in ethanol (6.0 mL), hydrazine hydrate (0.20 g, 4.0 mmol) was added at room temperature and stirring was continued under reflux for 2 hours. After cooling to the room temperature, the precipitate was filtered, washed with ethanol and dried under vacuum to give the title compound. Yield: 159 mg (87%). Melting Point 292-3° C. ¹H NMR (400 MHz, DMSO-d₆): δ 13.88 (br s, 1H), 8.44 (br s, 1H), 7.73 (d, 1H), 7.61 (m, 2H), 4.75 (m, 1H), 4.15 (t, 1H), 3.78 (m, 1H), 3.70 (m, 2H), 3.41 (t, 2H), 3.11 (m, 2H), 1.82 (s, 3H). ESMS: m/z 391 (M+1). C17H18N4O5S. Calcd, %: C, 52.30; H, 4.65; N, 14.35. Found, %: C, 52.39; H, 4.55; N, 14.36.

### EXAMPLE 16

### (S)—N-[3-(2-Amino-5,5-dioxo-6,7-dihydro-5H-516-thia-1,3-diaza-dibenzo[a,c]cyclohepten-9-yl)-2-oxo-oxazolidin-5-ylmethyl]-acetamide 16-1

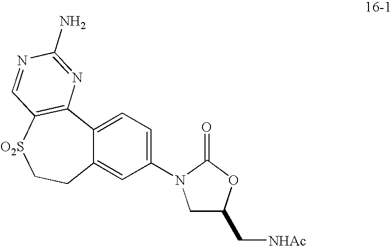

To a stirred suspension of (S)—N-[3-(2-Dimethylaminomethylene-1,3,3-trioxo-2,3,4,5-tetrahydro-1H-316-benzo[d]thiepin-7-yl)-2-oxo-oxazolidin-5-ylmethyl]-acetamide 14-1 (250 mg, 0.59 mmol) in ethanol (10.0 mL), guanidine hydrochloride (564 mg 5.9 mmol) was added, followed by K₂CO₃ (815 mg, 5.9 mmol), and stirring was continued under reflux for 4 hours. After cooling to the room temperature, the solids were filtered and suspended in water (10.0 mL). After stirring at room temperature for 1 hour, the precipitate was filtered, washed with water and dried under vacuum to give the title compound. Yield: 215 mg (87 %). Melting Point 211-213° C. (dec). ¹H NMR (400 MHz, DMSO-d₆) δ: 8.58 (s, 1H), 8.24 (t, 1H), 7.78 (m, 1H), 7.70-7.52 (m, 3H), 4.75 (m, 1H), 4.15 (t, 1H), 3.78 (m, 3H), 3.42 (t, 2H), 3.06 (t, 2H), 1.83 (s, 3H). ESMS: m/z 418 (M+1). Elemental Analysis for C18H19N5O5S.H2O. Calcd, %: C, 49.64; H, 4.86; N, 16.08. Found, %: C, 49.17; H, 4.81; N, 15.92.

### EXAMPLE 17

### (S)—N-[3-(4,4-Dioxo-5,6-dihydro-4H-1-oxa-416-thia-2-aza-benzo[e]azulen-8-yl)-2-oxo-oxazolidin-5-ylmethyl]-acetamide 17-1

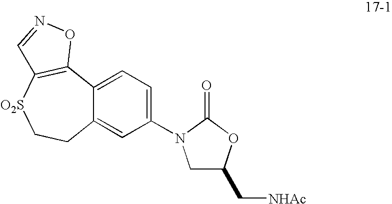

To a stirred suspension of (S)—N-[3-(2-dimethylaminomethylene-1,3,3-trioxo-2,3,4,5-tetrahydro-1H-316-benzo[d]thiepin-7-yl)-2-oxo-oxazolidin-5-ylmethyl]-acetamide 14-1 (200 mg, 0.47 mmol) in methanol (8.0 mL), a solution of hydroxylamine O-sulfonic acid (70 mg, 0.62 mmol) in methanol (1.0 mL) was added at room temperature and stirring was continued under reflux for 4 hours. After cooling to the room temperature, the precipitate was filtered, dried, and then chromatographed on silica gel (ethyl acetate-methanol 10:1) to give the title compound. Yield: 84 mg (53%). Melting Point 211-212° C. ¹H NMR (400 MHz, DMSO-d₆) δ: 9.39 (s, 1H), 8.25 (t, 1H), 8.04 (d, 1H), 7.78 (dd, 1H), 7.63 (d, 1H), 4.75 (m, 1H), 4.18 (t, 1H), 3.79 (m, 3H), 3.41 (m, 4H), 1.82 (s, 3H). ESMS: m/z 392 (M+1). C17H17N3O6S. Calcd, %: C, 52.17; H, 4.38; N, 10.47. Found, %: C, 52.33; H, 4.51; N, 10.55.

### EXAMPLE 18

### (S)—N-[3-(3-Methylsulfanyl-4,5-dihydro-1H-6-oxa-1,2-diaza-benzo[e]azulen-8-yl)-2-oxo-oxazolidin-5-ylmethyl]-acetamide 18-1

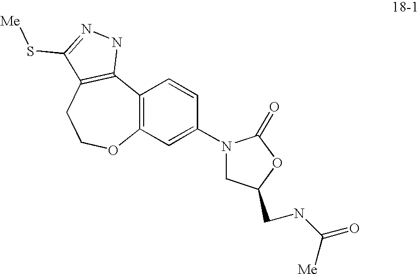

A solution of 0.50 g (1.57 mmol) of (S)—N-[2-oxo-3-(5-oxo-2,3,4,5-tetrahydro-benzo[b]oxepin-8-yl)-oxazolidin-5-ylmethyl]-acetamide 7-11, 0.11 mL (0.14 g, 1.88 mmol) of carbon disulfide, 0.21 mL (0.49 g, 3.5 mmol) of iodomethane, and THF (5 mL) was cooled in an ice bath under nitrogen and then treated dropwise with 4.4 mL of 1.0 M lithium hexamethyl disilazide in toluene (addition was complete in 5 minutes.) The mixture was allowed to warm to room temperature and stirred overnight; the suspension was then treated with aqueous saturated ammonium chloride and extracted with EtOAc. The organic layer was washed with brine, dried (MgSO4), and concentrated. The product was chromatographed over silica gel, eluting with 5% MeOH in EtOAc. MS (APCI) AP+, 423.0.

A solution of 0.33 g of the intermediate was dissolved in EtOH (heated until all solids had dissolved) and treated with 3.06 g (3.9 mmol) of hydrazine hydrate. The mixture was stirred overnight at room temperature and then refluxed for 2 hours. The suspension was cooled in an ice bath and filtered. The solids were washed with cold EtOH and dried to give the title compound. MS (APCI) AP+, 389.1. ¹H NMR (CDCl₃): δ 1.78 (s, 3 H), 2.44 (s, 3 H), 2.81 (m, 2 H), 3.35 (t, 2 H), 3.67-3.71 (m, 1 H), 4.07 (br t, 1 H), 4.19 (m, 2 H), 4.66 (m, 1 H), 7.23 (m, 2 H), 7.81 (m, 1 H), 8.20 (m, 1 H).

### EXAMPLE 19

### General procedure for 3-Amino-substituted pyrazoles (S)-4-{8-[5-(Acetylamino-methyl)-2-oxo-oxazolidin-3-yl]-4,5-dihydro-1H-6-oxa-1,2-diaza-benzo[e]azulen-3-yl}-piperazine-1-carboxylic acid tert-butyl

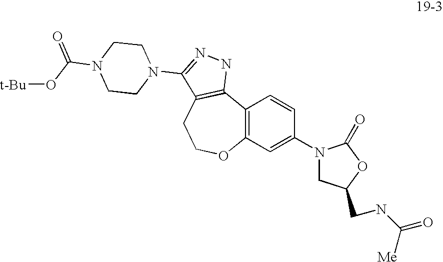

### Bromination of the oxazolidinone ketone (19-1) (Step 1)

A solution of 0.32 g (1.01 mmol) of (S)—N-[2-oxo-3-(5-oxo-2,3,4,5-tetrahydro-benzo[b]oxepin-8-yl)-oxazolidin-5-ylmethyl]-acetamide 7-11 in dichloromethane (30 mL) and acetic acid (5 mL) was treated with 0.35 g (1.1 mmol) of pyridinium tribromide and stirred at room temperature for 18 hours. The solution was poured into a large separatory funnel and then treated (cautiously) with saturated aqueous sodium bicarb. When all gas evolution ceased, the organic solution was washed with brine and dried over magnesium sulfate. Concentration gave the bromoketone intermediate.

### Preparation of thiosemicarbazide (19-2) (Step 2)

A solution of 0.37 g (2.0 mmol) of 4-methyl-4-phenyl-3-thiosemicarbazide (Rutter et. al., U.S. Pat. No. 4,282,031), 0.38 g (2.0 mmol) of N-t-butoxycarbonyl piperazine, and acetonitrile (15 mL) was refluxed for 5.0 hours, and then stirred at room temperature overnight. The solution was cooled to −40 ° C. and then stirred for 1.5 hours. The solids that formed were filtered and washed with acetonitrile and cold ether; air-drying of the solid gave the title compound. (J. Med Chem. 1997, 40, 2374-2385). MS (APCI): AP+, 261.1.

### Reaction of bromoketone and thiosemicarbazide (19-3) (Step 3)

A suspension of 0.29 g (0.73 mmol) of the bromo compound, 0.19 g (0.73 mmol) of the thiosemicarbazide, and ethanol (7 mL) was refluxed for 7 hours. The solution was cooled to room temperature and concentrated. The residue was dissolved in EtOAc, washed with aqueous sodium bicarbonate and brine, and dried (MgSO₄). Concentration gave the crude product which was chromatographed over silica gel, eluting with 95:5 dichloromethane:MeOH and then with 95:5 dichloromethane:MeOH containing 10% NH₃. MS (APCI) AP+, 527.2. ¹H NMR (CDCl₃) δ:1.47 (s, 9 H), 2.01 (s, 3 H), 2.93 (m, 2 H), 3.10 (m, 4 H), 3.55 (m, 4 H), 3.63-3.78 (m, 3 H), 4.02 (t, 1 H), 4.24-4.29 (m, 2 H), 4.8 (m, 1 H), 6.2 (m, 1 H), 7.15 (m, 1 H), 7.33 (m, 1 H), 7.49 (d, 1 H).

### EXAMPLE 20

### (S)—N-[3-(3-Morpholin-4-yl-4,5-dihydro-1H-6-oxa-1,2-diaza-benzo[e]azulen-8-yl)-2-oxo-oxazolidin-5-ylmethyl]-acetamide 20-1

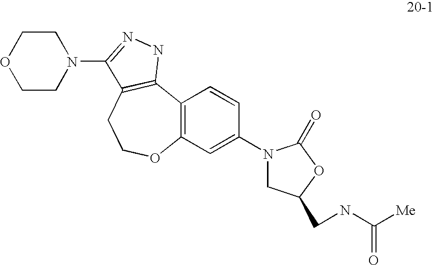

The title compound was prepared in a fashion analogous to Example 19 using 0.40 g (1.24 mmol) of the bromo intermediate and 0.20 g (1.2 mmol) of the thiosemicarbazide prepared from 4-methyl-4-phenyl-3-thiosemicarbazide and morpholine. The final product was purified via silica gel chromatography, eluting with 5% MeOH (containing 10% NH₃) in dichloromethane. MS (APCI) AP+, 428.2. ¹H NMR (CDCl₃): δ 2.02 (s, 3 H), 2.95 (t, 2 H), 3.16 (m, 4 H), 3.55-3.75 (m, 4 H), 3.85 (m, 4 H), 4.05 (t, 1 H), 4.28 (m, 2 H), 4.79 (m, 1 H), 6.06 (m, 1 H), 7.16 (d, 1 H), 7.38 (m, 1 H), 7.50 (m, 1 H).

### EXAMPLE 21

### (S)—N-{3-[3-(4-Fluoro-phenyl)-1,4,5,6-tetrahydro-1,2-diaza-benzo[e]azulen-9-yl]-2-oxo-oxazolidin-5-ylmethyl}-acetamide 20-2

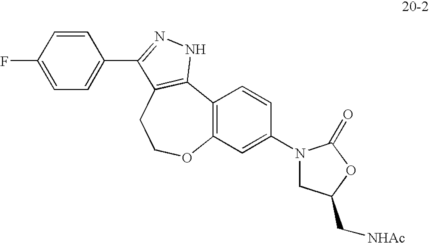

### (S)—N-{3-[4-(4-Fluoro-benzoyl)-5-oxo-2,3,4,5-tetrahydro-benzo[b]oxepin-8-yl]-2-oxo-oxazolidin-5-ylmethyl}-acetamide (80474×36) (Step 1)

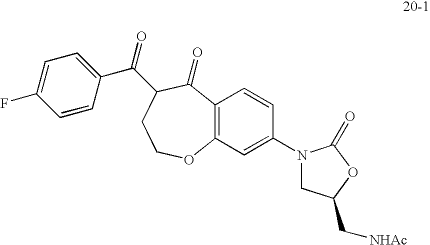

The title compound was prepared according to general method AA using (S)—N-[2-oxo-3-(5-oxo-2,3,4,5-tetrahydro-benzo[b]oxepin-8-yl)-oxazolidin-5-ylmethyl]-acetamide 7-11 (0.30 g, 0.94 mmol), THF (10 mL), LDA (2 M, 2.4 equiv., 1.13 mL) , and 4-fluorobenzoyl chloride (0.149 g, 1.0 equiv., 1.58 mmol). The isolated residue was subjected to silica gel flash chromatography, eluting with MeOH/dichloromethane gradient (0-6% MeOH over 1½ hours) to afford the title compound. Isolated yield: 24%. MS-APCI (m/z+): 397, 441.

### (S)—N-{3-[3-(4-Fluoro-phenyl)-1,4,5,6-tetrahydro-1,2-diaza-benzo[e]azulen-9-yl]-2-oxo-oxazolidin-5-ylmethyl}-acetamide (20-2) (Step 2)

The title compound was prepared according to general method DD using (S)—N-{3-[4-(4-fluoro-benzoyl)-5-oxo-2,3,4,5-tetrahydro-benzo[b]oxepin-8-yl]-2-oxo-oxazolidin-5-ylmethyl }-acetamide (0.09 g, 0.20 mmol) and hydrazine hydrate (0.029 g, 0.92 mmol, 4.5 equiv.) in EtOH (4 mL). The isolated residue was subjected to silica gel flash chromatography, eluting with MeOH/dichloromethane gradient (0-7% MeOH over 70 minutes) to afford the title compound. Isolated yield: 0.038 g (43%). ¹H NMR (400 MHz, DMSO-d₆): δ 13.24 (br s, 1H), 8.22 (m, 1H), 7.93 (d, 1H), 7.67 (br m, 2H), 7.33-7.20 (m, 4H), 4.70 (m, 1H), 4.23 (m, 2H), 4.11 (t, 1H), 3.73 (t, 1H), 3.39 (m, 2 H), 3.09 (m, 2H), 1.81 (s, 3H); MS-APCI (m/z+): 393, 437.

### EXAMPLE 22

### (S)—N-[2-Oxo-3-(3-pyridin-4-yl-4,5-dihydro-1H-6-oxa-1,2-diaza-benzo[e]azulen-8-yl)-oxazolidin-5-ylmethyl]-acetamide

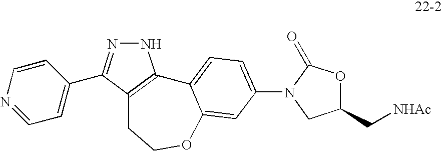

### (S)—N-{2-Oxo-3-[5-oxo-4-(pyridine-4-carbonyl)-2,3,4,5-tetrahydro-benzo[b]oxepin-8-yl]-oxazolidin-5-ylmethyl}-acetamide (22-1) (Step 1)

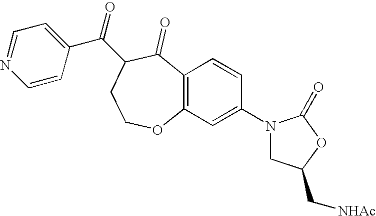

The title compound was prepared according to general method GG using (S)—N-[2-oxo-3-(5-oxo-2,3,4,5-tetrahydro-benzo[b]oxepin-8-yl)-oxazolidin-5-ylmethyl]-acetamide (7-11) (M) (0.30 g, 0.94 mmol), THF (10 mL), lithium t-butoxide (3.1 equiv., 2.92 mL) and isonicotinoyl chloride hydrochloride (1.2 equiv., 0.20 g, 1.13 mmol). The isolated residue was subjected to silica gel flash chromatography, eluting with MeOH/dichloromethane gradient (0-7% MeOH over 70 minutes) to afford the title compound. Isolated yield: 28%. MS-APCI (m/z+): 380, 424.

### (S)—N-[2-Oxo-3-(3-pyridin-4-yl-4,5-dihydro-1H-6-oxa-1,2-diaza-benzo[e]azulen-8-yl)-oxazolidin-5-ylmethyl]-acetamide (22-2) (Step 2)

The title compound was prepared according to general method DD using (S)—N-{2-oxo-3-[5-oxo-4-(pyridine-4-carbonyl)-2,3,4,5-tetrahydro-benzo[b]oxepin-8-yl]-oxazolidin-5-ylmethyl}-acetamide (0.11 g, 0.26 mmol) and hydrazine hydrate (0.037 g, 1.15 mmol, 4.5 equiv.) in EtOH (5 mL). The isolated residue was subjected to silica gel flash chromatography, eluting with MeOH/dichloromethane gradient (2-8% MeOH over 1H.) to afford the title compound. Isolated yield: 0.065 g (60%). ¹H NMR (400 MHz, DMSO-d₆): δ 13.60-13 50 (br s+br s, 1H), 8.61 (m, 2H), 8.22 (t, 1H), 7.95 (d, 1H), 7.72 (m, 1H), 7.63 (m, 1H), 7.30 (s, 1H), 7.23 (m, 1H), 4.71 (m, 1H), 4.26 (t, 2H), 4.13 (t, 1H), 3.75 (dd 1H), 3.41-3.38 (m, 2 H), 3.28-3.15 (m, 2H), 1.82 (s, 3H); MS-APCI (m/z+): 376, 420.

### EXAMPLE 23

### (S)—N-[2-Oxo-3-(3-phenyl-4,5-dihydro-1H-6-oxa-1,2-diaza-benzo[e]azulen-8-yl)-oxazolidin-5-ylmethyl]-acetamide

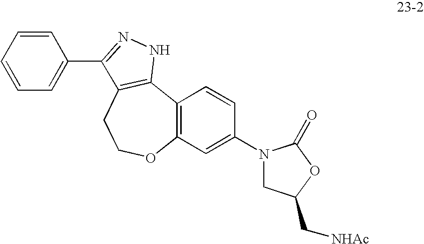

### (S)—N-[3-(4-Benzoyl-5-oxo-2,3,4,5-tetrahydro-benzo[b]oxepin-8-yl)-2-oxo-oxazolidin-5-ylmethyl]-acetamide (84249×18) (23-1) (Step 1)

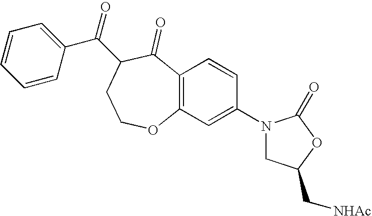

The title compound was prepared according to general method CC using (S)—N-[2-oxo-3-(5-oxo-2,3,4,5-tetrahydro-benzo[b]oxepin-8-yl)-oxazolidin-5-ylmethyl]-acetamide 7-11 (0.30 g, 0.94 mmol), THF (8 mL), LiHMDS (1 M in THF, 2.0 equiv., 1.89 mL) and a solution of benzoyl chloride (1.05 equiv., 0.14 g, 0.99 mmol) in THF (2 mL). The isolated residue was subjected to silica gel flash chromatography, eluting with MeOH/dichloromethane gradient (0-6% MeOH over 70 minutes) to afford the title compound. Isolated yield: 58%. MS-APCI (m/z+): 379, 423.

### (S)—N-[2-Oxo-3-(3-phenyl-4,5-dihydro-1H-6-oxa-1,2-diaza-benzo[e]azulen-8-yl)-oxazolidin-5-ylmethyl]-acetamide (23-2) (Step 2)

To (S)—N-[3-(4-benzoyl-5-oxo-2,3,4,5-tetrahydro-benzo[b]oxepin-8-yl)-2-oxo-oxazolidin-5-ylmethyl]-acetamide (7-11) (0.23 g, 0.54 mmol) in acetic acid (12 mL) was added hydrazine hydrate (0.087 g, 2.72 mmol, 5.0 equiv.) and the mixture was heated to 100° C. After 3 h, the mixture was cooled to room temperature and the solvent was removed in vacuo. Saturated sodium bicarbonate was added and the aqueous layer was extracted with EtOAc. The combined organic layers were washed with brine, dried over MgSO4, filtered and concentrated in vacuo. The residue was triturated with EtOAc. The resulting solid was collected by filtration and washed with EtOAc and Et₂O to afford the title compound. Isolated yield: 0.100 g (44%). ¹H NMR (400 MHz, DMSO-d₆): δ 13.20 (br s, 1H), 8.22 (m, 1H), 7.64 (m, 2H), 7.45 (m, 3H), 7.36 (m, 1H), 7.26 (m, 2H), 4.70 (m, 1H), 4.24 (m, 2H), 4.11 (t, 1H), 3.74 (dd 1H), 3.39 (m, 2 H), 3.12 (m, 2H), 1.81 (s, 3H); MS-APCI (m/z+): 375, 419.

### EXAMPLE 24

### (S)—N-[3-(3-Isoxazol-5-yl-4,5-dihydro-1H-6-oxa-1,2-diaza-benzo[e]azulen-8-yl)-2-oxo-oxazolidin-5-ylmethyl]-acetamide

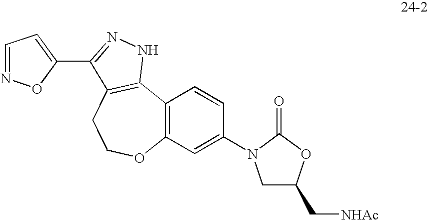

### (S)—N-{3-[4-(Isoxazole-5-carbonyl)-5-oxo-2,3,4,5-tetrahydro-benzo[b]oxepin-8-yl]-2-oxo-oxazolidin-5-ylmethyl}-acetamide (24-1) (Step 1)

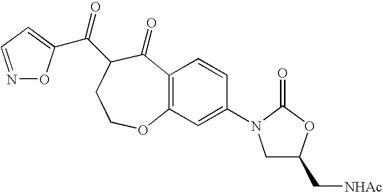

The title compound was prepared according to general method CC using (S)—N-[2-oxo-3-(5-oxo-2,3,4,5-tetrahydro-benzo[b]oxepin-8-yl)-oxazolidin-5-ylmethyl]-acetamide (7-11) (0.50 g, 1.57 mmol), THF (14 mL), LiHMDS (1 M in THF, 2.1 equiv., 3.30 mL) and a solution of 1,5-Dimethyl-1H-pyrazole-3-carbonyl chloride (1.2 equiv., 0.250 g, 1.90 mmol) in THF (8 mL). The isolated residue was subjected to silica gel flash chromatography, eluting with MeOH/dichloromethane gradient (0-7% MeOH over 1 H and 10 minutes) to afford the title compound. Isolated yield: 26%. MS-APCI (m/z+): 360, 414.

### (S)—N-[3-(3-Isoxazol-5-yl-4,5-dihydro-1H-6-oxa-1,2-diaza-benzo[e]azulen-8-yl)-2-oxo-oxazolidin-5-ylmethyl]-acetamide (24-2) (Step 2)

The title compound was prepared according to general method CC using (S)—N-{3-[4-(isoxazole-5-carbonyl)-5-oxo-2,3,4,5-tetrahydro-benzo[b]oxepin-8-yl]-2-oxo-oxazolidin-5-ylmethyl}-acetamide (0.360 g, 0.87 mmol) and hydrazine hydrate (0.070 g, 2.18 mmol, 2.5 equiv.) in EtOH (18 mL). The isolated residue was triturated with dichloromethane and a trace amount of MeOH. The resulting solid was collected by filtration and washed successively with EtOAc, dichloromethane and a trace amount of MeOH to afford the title compound. Isolated yield: 0.140 g (39%). ¹H NMR (400 MHz, DMSO-d₆) δ: 13.63 (br s, 1H), 8.60 (s, 1H), 8.19 (t, 1H), 7.96 (br s, 1H), 7.30-7.26 (d+s, 2H), 6.74 (s, 1H), 4.67 (m, 1H), 4.26 (m, 2H), 4.09 (t, 1H), 3.72 (dd 1H), 3.31 (m, partially obscured by H₂O, 2H), 3.16 (m, 2H), 1.79 (s, 3H); MS-APCI (m/z+): 366, 410.

### EXAMPLE 25

### (S)—N-[3-(3-Methylamino-4,5-dihydro-1H-6-oxa-1,2-diaza-benzo[e]azulen-8-yl)-2-oxo-oxazolidin-5-ylmethyl]-acetamide

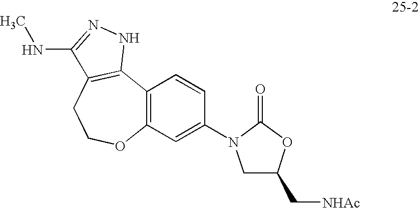

### (S)—N-[3-(4-Bromo-5-oxo-2,3,4,5-tetrahydro-benzo[b]oxepin-8-yl)-2-oxo-oxazolidin-5-ylmethyl]-acetamide (25-1) (Step 1)

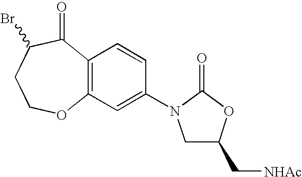

To N-[2-oxo-3-(5-oxo-2,3,4,5-tetrahydro-benzo[b]oxepin-8-yl)-oxazolidin-5-ylmethyl]-acetamide 7-11 (2.0 g, 6.3 mmol) in 210 mL of dichloromethane was added 35 mL of glacial acetic acid. Pyridinium bromide perbromide (2.2 g, 6.9 mmol) was added in one portion and the solution was stirred for 3 h. The solution was concentrated in vacuo to give a dark oil. The oil was diluted with dichloromethane and washed with water (twice), sat NaHCO₃, dried over Na₂SO₄, and concentrated in vacuo. Purification by silica gel chromatography afforded the title compound in quantitative yield (2.50 g). MS-APCI (m/z+): 397, 398 (M+H).

### (S)—N-[3-(3-Methylamino-4,5-dihydro-1H-6-oxa-1,2-diaza-benzo[e]azulen-8-yl)-2-oxo-oxazolidin-5-ylmethyl]-acetamide (25-2) (Step 2)

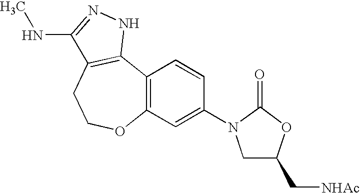

Following general procedure JJ, (S)—N-[3-(4-bromo-5-oxo-2,3,4,5-tetrahydro-benzo[b]oxepin-8-yl)-2-oxo-oxazolidin-5-ylmethyl]-acetamide (0.406 g, 1 mmol) and 4-methylthiosemicarbazide (0.108 g, 1 mmol) gave the title compound. MS-APCI (m/z+): 328, 372 (M+H). ¹H NMR (400 MHz, DMSO-d₆) δ: 11.69 (s, 1H), 8.23 (t, 1H), 7.78 (d, 1H;), 7.25 (m, 2H), 4.83 (m, 1H), 4.71 (m, 1H), 4.21 (t, 1H), 4.11 (m, 1H), 3.74 (dd, 1H), 3.40 (t, 2H), 2.72 (m, 4H), 1.83 (s, 3H). MS-APCI (m/z+): 328, 372 (M+H).

### EXAMPLE 26

### (S)—N-[3-(3-Ethylamino-4,5-dihydro-1H-6-oxa-1,2-diaza-benzo[e]azulen-8-yl)-2-oxo-oxazolidin-5-ylmethyl]-acetamide 26-1

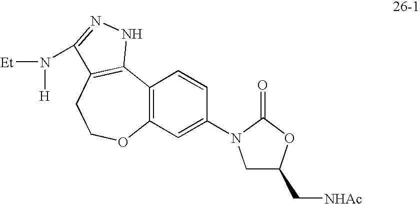

Following general procedure EE, (S)—N-[3-(4-bromo-5-oxo-2,3,4,5-tetrahydro-benzo[b]oxepin-8-yl)-2-oxo-oxazolidin-5-ylmethyl]-acetamide (0.395 g, 1 mmol) and 4-ethylthiosemicarbazide (0.119 g, 1 mmol) gave the title compound in 13% yield (0.048 g). ¹H NMR (400 MHz, DMSO-d₆) δ: 8.23 (t, 1H), 7.82 (d, 1H), 7.24 (m, 2H), 4.81 (t, 1H), 4.71 (m, 1H), 4.21 (t, 2H), 4.11 (t, 1H), 3.74 (dd, 1H), 3.40 (t, 2H), 3.14 (m, 2H), 2.72 (t, 2H), 1.83 (s, 3H), 1.15 (t, 3H). MS-APCI (m/z+): 342, 386 (M+H).

### EXAMPLE 27

### (S)—N-[2-Oxo-3-(3-phenylamino-4,5-dihydro-1H-6-oxa-1,2-diaza-benzo[e]azulen-8-yl)-oxazolidin-5-ylmethyl]-acetamide 27-1

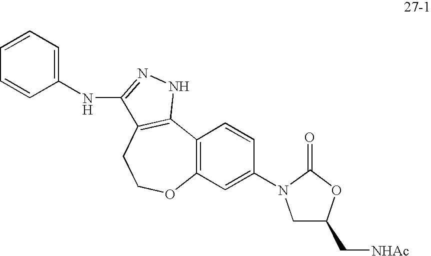

A mixture of (S)—N-[3-(4-bromo-5-oxo-2,3,4,5-tetrahydro-benzo[b]oxepin-8-yl)-2-oxo-oxazolidin-5-ylmethyl]-acetamide (0.199 g, 0.5 mmol), 4-phenyl-3-thiosemicarbazide (0.083 g, 0.5 mmol), 0.15 mL concentrated HCl, and 2.3 mL of absolute EtOH were heated to 78° C. for 3 h. The mixture was cooled to room temperature and filtered. The filtrate was concentrated in vacuo and purifed by silica gel chromatography to give the title compound in 53% yield (0.155 g). ¹H NMR (400 MHz, DMSO-d₆): δ 12.37 (s, 1H), 8.26 (t, 1H), 7.88 (d, 1H), 7.84 (s, 1H), 7.32 (m, 4H), 7.16 (t, 2H), 6.70 (t, 1H), 4.72 (m, 1H), 4.26 (m, 2H), 4.13 (t, 1H), 3.75 (dd, 1H), 3.41 (m, 2H), 2.87 (m, 2H), 1.84 (s, 3H). MS-APCI (m/z+): 390, 434 (M+H).

### EXAMPLE 28

### (S)—N-{3-[3-(4-Methoxy-benzylamino)-4,5-dihydro-1H-6-oxa-1,2-diaza-benzo[e]azulen-8-yl]-2-oxo-oxazolidin-5-ylmethyl}-acetamide 28-1

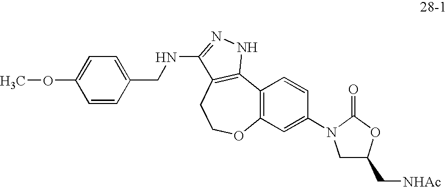

Following general procedure EE, (S)—N-[3-(4-bromo-5-oxo-2,3,4,5-tetrahydro-benzo[b]oxepin-8-yl)-2-oxo-oxazolidin-5-ylmethyl]-acetamide (0.395 g, 1.0 mmol) and 4-(4-methoxybenzyl)-3-thiosemicarbazide (0.211 g, 0.51 mmol) gave the title compound in 23% yield (0.108 g). ¹H NMR (400 MHz, DMSO-d₆): δ 11.72 (s, 1H), 8.23 (t, 1H), 7.76 (d, 1H), 7.31 (d, 2H), 7.24 (m, 2H), 6.86 (d, 2H), 5.34 (br s, 1H), 4.71 (m, 1H), 4.24 (m, 4H), 4.14 (t, 1H), 3.74 (m, 1H), 3.72 (s, 3H), 3.40 (m, 2H), 2.76 (m, 2H), 1.83 (s, 3H). MS-APCI (m/z+): 478, 434 (M+H).

### EXAMPLE 29

### (S)—N-[3-(3-Amino-4,5-dihydro-1H-6-oxa-1,2-diaza-benzo[e]azulen-8-yl)-2-oxo-oxazolidin-5-ylmethyl]-acetamide 29-1

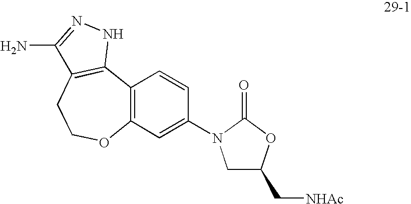

To a solution of (S)—N-{3-[3-(4-methoxy-benzylamino)-4,5-dihydro-1H-6-oxa-1,2-diaza-benzo[e]azulen-8-yl]-2-oxo-oxazolidin-5-ylmethyl}-acetamide (0.244 g, 0.511 mmol) in 10 mL of dichloromethane was added triethylsilane (98 mL, 0.613 mmol), followed by the dropwise addition of trifluoroacetic acid (1.0 mL, 12.77 mmol). The mixture was stirred at room temperature overnight and then concentrated in vacuo. The residue was diluted with EtOAc and then concentrated in vacuo a second time. The solid residue was dissolved in 10% MeOH/dichloromethane+2% NH₄OH and allowed to stir overnight. The mixture was concentrated in vacuo, diluted with EtOAc and then concentrated in vacuo to give a solid. The solid was washed with 75 mL of hexanes which had been heated to reflux. The solid was dissolved in 10% MeOH/dichloromethane+2% NH₄OH and then purifed by silica gel chromatography to give the title compound in 80% yield (0.145 g). ¹H NMR (400 MHz, CD₃OD): δ 7.74 (br s, 1H), 7.32 (d, 1H), 7.26 (d, 1H), 4.79 (m, 1H), 4.29 (t, 2H), 4.15 (t, 1H), 3.83 (dd, 1H), 3.57 (d, 2H), 2.85 (t, 2H), 1.97 (s, 3H). MS-APCI (m/z+): 314, 358 (M+H).

### EXAMPLE 30

### (S)—N-[3-(3-Ethyl-4,5-dihydro-1H-6-oxa-1,2-diaza-benzo[e]azulen-8-yl)-2-oxo-oxazolidin-5-ylmethyl]-acetamide 30-2

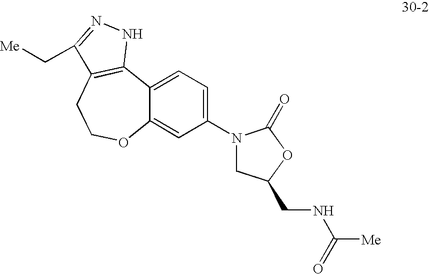

### (S)—N-[2-Oxo-3-(5-oxo-4-propionyl-2,3,4,5-tetrahydro-benzo[b]oxepin-8-yl)-oxazolidin-5-ylmethyl]-acetamide (30-1) (Step 1)

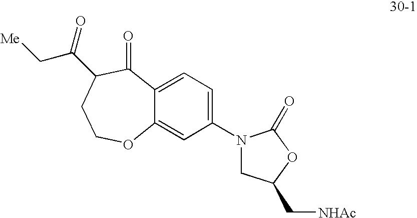

A solution of (S)—N-[2-oxo-3-(5-oxo-2,3,4,5-tetrahydro-benzo[b]oxepin-8-yl)-oxazolidin-5-ylmethyl]-acetamide 7-11 (500 mg, 1.57 mmol) in THF (15 mL) was cooled to 0° C. and a 1 M solution of LiHMDS in THF (3.15 mL, 3.15 mmol) was added. The mixture was stirred at 0° C. for 30 min, and then a solution of propionyl chloride (144 mL, 1.65 mmol) in THF (10 mL) was added dropwise over 1 h. After warming to room temperature overnight, the mixture was treated with saturated ammonium chloride and extracted with dichloromethane. The organic phase was dried over Na₂SO₄ and concentrated in vacuo. The oil was subjected to silica gel chromatography to give the title compound. Isolated yield: 222 mg (38%). MS-APCI (m/z+): 331, 375 (M+H).

### (S)—N-[3-(3-Ethyl-4,5-dihydro-1H-6-oxa-1,2-diaza-benzo[e]azulen-8-yl)-2-oxo-oxazolidin-5-ylmethyl]-acetamide (30-2) (Step 2)

To a solution of (S)—N-[2-oxo-3-(5-oxo-4-propionyl-2,3,4,5-tetrahydro-benzo[b]oxepin-8-yl)-oxazolidin-5-ylmethyl]-acetamide (222 mg, 0.595 mmol) in ethanol (11 mL) was added hydrazine hydrochloride (48.9 mg, 71.3 mmol). The reaction mixture was then refluxed overnight. The solution was cooled to room temperature and sodium bicarbonate was added. The aqueous layer was extracted with dichloromethane, and the organic layer was then dried over Na₂SO₄ and concentrated in vacuo. The resultant oil was diluted with EtOAc which caused a solid to precipitate from the solution. The solid was collected and purified by silica gel chromatography. Isolated yield: 23 mg (10%). ¹H NMR (400 MHz, CDCl₃): δ: 7.72 (d, 1H), 7.20-7.24 (m, 1H), 7.16 (d, 1H), 4.71-4.74 (m, 1H), 4.25 (t, 2H), 4.06 (t, 1H), 3.75 (dd, 1H), 3.51 (t, 2H), 2.90 (t, 2H), 2.58 (q, 2H), 1.93 (s, 3H), 1.19 (t, 3H); MS-APCI (m/z+): 327, 371 (M+H).

### EXAMPLE 31

### (S)—N-{3-[3-(5-Methyl-isoxazol-3-yl)-4,5-dihydro-1H-6-oxa-1,2-diaza-benzo[e]azulen-8-yl]-2-oxo-oxazolidin-5-ylmethyl}-acetamide

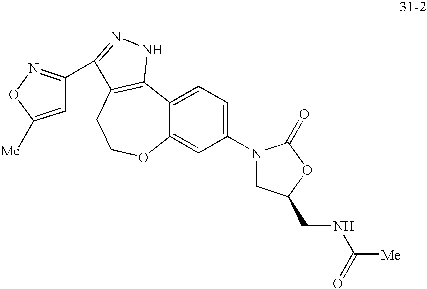

### (S)—N-{3-[4-(5-Methyl-isoxazole-3-carbonyl)-5-oxo-2,3,4,5-tetrahydro-benzo[b]oxepin-8-yl]-2-oxo-oxazolidin-5-ylmethyl}-acetamide (31-1) (Step 1)

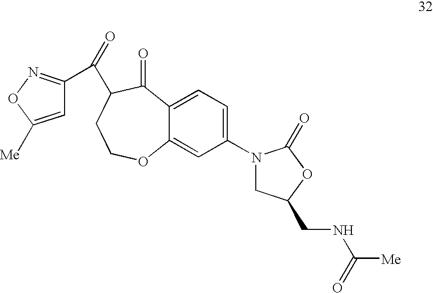

A solution of (S)—N-[2-oxo-3-(5-oxo-2,3,4,5-tetrahydro-benzo[b]oxepin-8-yl)-oxazolidin-5-ylmethyl]-acetamide 7-11 (458 mg, 1.44 mmol) in THF (15 mL) was cooled to 0° C. and a 1 M solution of LiHMDS in THF (3.02 mL, 3.02 mmol) was added. The mixture was stirred at 0° C. for 30 minutes then a solution of 5-methyl-isoxazole-3-carbonyl chloride (214 mg, 1.73 mmol) in THF (10 mL) was added dropwise over 1 h. After warming to room temperature overnight, the mixture was treated with saturated ammonium chloride and extracted with dichloromethane. The organic layer was dried over Na₂SO₄ and concentrated in vacuo. The resultant oil was subjected to silica gel chromatography to result in the title compound. Isolated yield: 388 mg (63%). MS-APCI (m/z+): 384, 428 (M+H).

### (S)—N-{3-[3-(5-Methyl-isoxazol-3-yl)-4,5-dihydro-1H-6-oxa-1,2-diaza-benzo[e]azulen-8-yl]-2-oxo-oxazolidin-5-ylmethyl}-acetamide (31-2) (Step 2)

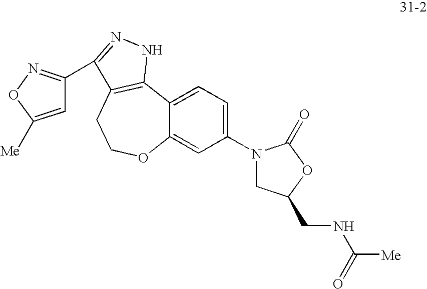

To a solution of (S)—N-{3-[4-(5-methyl-isoxazole-3-carbonyl)-5-oxo-2,3,4,5-tetrahydro-benzo[b]oxepin-8-yl]-2-oxo-oxazolidin-5-ylmethyl) -acetamide (388 mg, 0.909 mmol) in ethanol (16 mL) was added hydrazine hydrate (70.8 mL, 2.27 mmol). The reaction mixture was then stirred at room temperature overnight. The precipitate was collected by filtration and washed with ethanol. Isolated yield: 116 mg (30%). ¹H NMR (400 MHz, CD₃OD): δ 7.75 (d, 1H), 7.66 (s, 1H), 7.21 (dd, 1H), 7.17 (d, 1H), 4.71-4.77 (m, 1H), 4.36 (ddd, 1H), 4.29 (t, 1H), 4.05-4.13 (m, 1H), 3.78 (dq, 2H), 3.51-3.54 (m, 2H), 2.39 (s, 3H), 1.92 (s, 3H); MS-APCI (m/z+): 380,424 (M+H).

### EXAMPLE 32

### (S)—N-[3-(3-Furan-2-yl-4,5-dihydro-1H-6-oxa-1,2-diaza-benzo[e]azulen-8-yl)-2-oxo-oxazolidin-5-ylmethyl]-acetamide 32-2

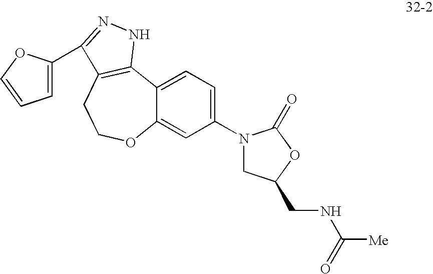

### (S)—N-{3-[4-(Furan-2-carbonyl)-5-oxo-2,3,4,5-tetrahydro-benzo[b]oxepin-8-yl]-2-oxo-oxazolidin-5-ylmethyl}-acetamide (32-1) (Step 1)

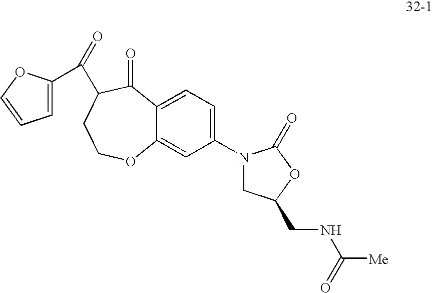

A solution of (S)—N-[2-oxo-3-(5-oxo-2,3,4,5-tetrahydro-benzo[b]oxepin-8-yl)-oxazolidin-5-ylmethyl]-acetamide 7-11 (500 mg, 1.57 mmol) in THF (15 mL) was cooled to 0° C. and a 1 M solution of LiHMDS in THF (3.30 mL, 3.30 mmol) was added. The mixture was stirred at 0° C. for 30 min and then a solution of furan-2-carbonyl chloride (246 mg, 1.89 mmol) in THF (10 mL) was added dropwise over 1 h. The mixture was treated with saturated ammonium chloride and extracted with dichloromethane. The organic layer was dried over Na₂SO₄ and concentrated in vacuo. The resultant oil was subjected to silica gel chromatography to give the title compound. Isolated yield: 343 mg (53%). MS-APCI (m/z+): 369, 413 (M+H).

### (S)—N-[3-(3-Furan-2-yl-4,5-dihydro-1H-6-oxa-1,2-diaza-benzo[e]azulen-8-yl)-2-oxo-oxazolidin-5-ylmethyl]-acetamide (32-2) (Step 2)

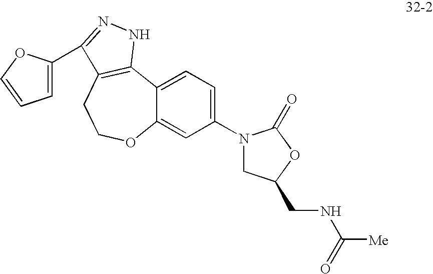

To a solution of (S)—N-{3-[4-(furan-2-carbonyl)-5-oxo-2,3,4,5-tetrahydro-benzo[b]oxepin-8-yl]-2-oxo-oxazolidin-5-ylmethyl}-acetamide (343 mg, 0.831 mmol) in ethanol (13 mL) was added hydrazine hydrate (64.7 mL, 2.08 mmol). The reaction mixture was then stirred at room temperature overnight. The precipitate was collected by filtration and washed with ethanol. Isolated yield: 227 mg (67%). ¹H NMR (400 MHz, DMSO-d₆) δ: 8.19 (t, 1H), 7.98 (d, 1H), 7.73 (d, 1H), 7.18-7.34 (m, 2H), 6.84 (d, 1H), 4.67 (sext, 1H), 4.24 (t, 2H), 3.70 (dd, 1H), 3.36 (t, 2H), 3.00 (br s, 2H), 1.79 (s, 3H); MS-APCI (m/z+): 365, 409 (M+H).

### EXAMPLE 33

### (S)—N-[3-(2-Amino-4,5-dihydro-6-oxa-3-thia-1-aza-benzo[e]azulen-8-yl)-2-oxo-oxazolidin-5-ylmethyl]-acetamide hydrobromic acid salt 33-1

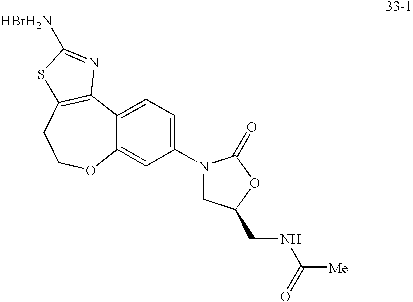

To (S)—N-[3-(4-Bromo-5-oxo-2,3,4,5-tetrahydro-benzo[b]oxepin-8-yl)-2-oxo-oxazolidin-5-ylmethyl]-acetamide (200.0 mg, 0.503 mmol) dissolved in warm ethanol (2.5 mL) was added thiourea (38.3 mg, 0.503 mmol). The mixture was heated in a microwave reactor to 100° C. for 3 minutes, resulting in the formation of an off-solid in the solution. The solid was collected by filtration, washed with ethanol (2 mL) and ethyl acetate (2 mL) and dried to afford the title compound. Isolated yield: 85.0 mg (37%). ¹H NMR (400 MHz, CD₃OD) δ: 8022 (t, 1H), 7.82 (d, 1H), 7.31 (dd, 1H), 7.28 (d, 1H), 4.71 (sext, 1H), 4.26 (t, 2H), 4.11 (t, 1H), 3.73 (dd, 1H), 3.39 (t, 2H), 3.09 (t, 2H), 1.801 (s, 3H); MS-APCI (m/z+): 331, 375 (M+H).

### EXAMPLE 34

### (S)—N-[3-(2-Methylamino-4,5-dihydro-6-oxa-3-thia-1-aza-benzo[e]azulen-8-yl)-2-oxo-oxazolidin-5-ylmethyl]-acetamide hydrobromic acid salt 34-1

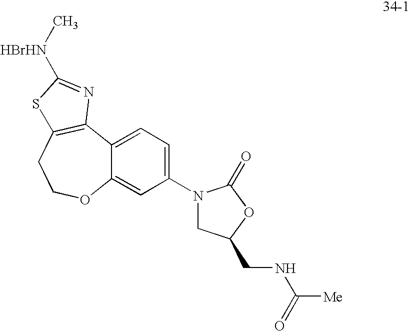

To (S)—N-[3-(4-Bromo-5-oxo-2,3,4,5-tetrahydro-benzo[b]oxepin-8-yl)-2-oxo-oxazolidin-5-ylmethyl]-acetamide (3) (170.1 mg, 0.428 mmol) dissolved in warm ethanol (2 mL) was added methyl thiourea (38.6 mg, 0.428 mmol). The resulting mixture was heated in a microwave reactor to 120° C. for 4 minutes, resulting in the formation of a solid which was suspended in a solution. The solid was collected by filtration and washed with ethanol (2 mL) and ethyl acetate (2 mL)to afford the title compound. Isolated yield: 98.8 mg (49%). ¹H NMR (400 MHz, CD₃OD) δ: 9.41 (br s, 1H), 7.91 (d, 1H), 7.41 (t, 1), 7.353 (s, 1H), 7.19 (d, 1H), 4.68 (sext, 1), 4.23 (t, 2), 3.93 (t, 1H), 3.71 (t, 1H), 3.48 (t, 2H), 3.05 (t, 2H), 3.01 (d, 3H), 1.87 (s, 3H); MS-APCI (m/z+): 345, 389 (M+H).

### EXAMPLE 35

### (S)—N-[2-Oxo-3-(5-oxo-4-pyridin-4-ylmethylene-2,3,4,5-tetrahydro-benzo[b]oxepin-8-yl)-oxazolidin-5-ylmethyl]-acetamide 35-1

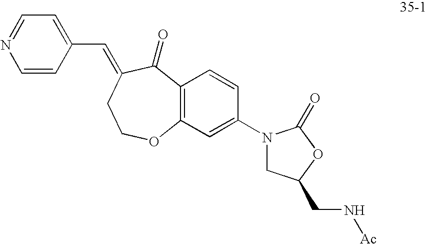

The title compound was prepared as described in the general procedure FF using (S)—N-[2-Oxo-3-(5-oxo-2,3,4,5-tetrahydro-benzo[b]oxepin-8-yl)-oxazolidin-5-ylmethyl]-acetamide 7-11 (0.316 g), 4-pyridinecarboxaldehyde (0.48 g), acetic acid (2 mL) and piperidine (2 mL). The crude product was purified by flash silica gel chromatography using dichloromethane/methanol as the eluting gradient system. Yield: 0.3 g. APCI (m/z): 408 (M+H).

### EXAMPLE 36

### (S)—N-[3-(4-Dimethylaminomethylene-5-oxo-2,3,4,5-tetrahydro-benzo[b]oxepin-8-yl)-2-oxo-oxazolidin-5-ylmethyl]-acetamide 36-1

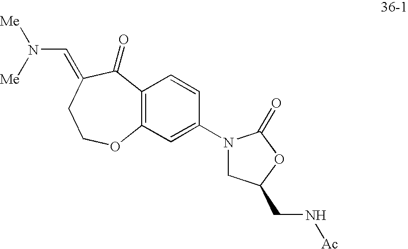

(S)—N-[2-Oxo-3-(5-oxo-2,3,4,5-tetrahydro-benzo[b]oxepin-8-yl)-oxazolidin-5-ylmethyl]-acetamide 7-11 (0.5 g) was dissolved in 15 mL of n-propanol. To the resulting mixture was added N,N-dimethylformamide dimethyl acetal (4 equiv.). The reaction mixture was stirred at reflux under an inert atmosphere for 16 h. The mixture was then cooled to room temperature and ether was added. The solid obtained was filtered and dried. APCI (m/z): 374 (M+H).

### EXAMPLE 37

### (S)—N-[3-(4,5-Dihydro-1H-6-oxa-1,2-diaza-benzo[e]azulen-8-yl)-2-oxo-oxazolidin-5-ylmethyl]-acetamide

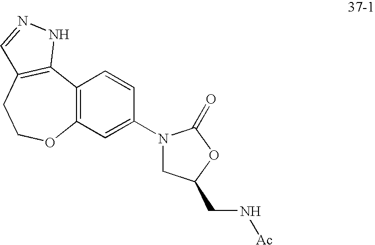

(S)—N-[3-(4-Dimethylaminomethylene-5-oxo-2,3,4,5-tetrahydro-benzo[b]oxepin-8-yl)-2-oxo-oxazolidin-5-ylmethyl]-acetamide (1.6 mmol), and hydrazine (0.2 g) were dissolved in ethanol (15 mL). The resulting reaction mixture was stirred at room temperature under an inert atmosphere for 20 hours. The mixture was concentrated in vacuo. The residue was purified by flash silica gel chromatography using dichloromethane/methanol as the eluting gradient system. Yield: 60%. APCI (m/z): 343 (M+H).

### EXAMPLE 38

### (S)—N-[3-(4,5-Dihydro-1,6-dioxa-2-aza-benzo[e]azulen-8-yl)-2-oxo-oxazolidin-5-ylmethyl]-acetamide 38-1

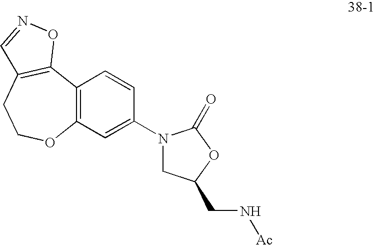

(S)—N-[3-(4-Dimethylaminomethylene-5-oxo-2,3,4,5-tetrahydro-benzo[b]oxepin-8-yl)-2-oxo-oxazolidin-5-ylmethyl]-acetamide 7-11 (0.5 g) was dissolved in dry methanol. The resulting mixture was cooled to 0° C. Hydroxylamine-O-sulfonic acid (1.1 eq) in methanol was added by dropwise addition. The reaction mixture was warmed to room temperature and stirred for 30 minutes. The reaction mixture was then treated with saturated sodium bicarbonate solution, extracted with dichloromethane and ethyl acetate, and dried over MgSO₄. The residue was purified by flash silica gel chromatography using dichloromethane/methanol as the eluting gradient system. Yield: 75%. APCI (m/z): 344 (M+H).

### EXAMPLE 39

### (S)—N-[3-(2-Methyl4,5-dihydro-2H-6-oxa-1,2-diaza-benzo[e]azulen-8-yl)-2-oxo-oxazolidin-5-ylmethyl]-acetamide 39-1

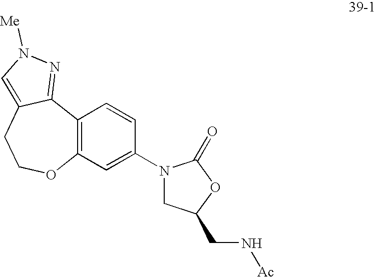

(S)—N-[3-(4-Dimethylaminomethylene-5-oxo-2,3,4,5-tetrahydro-benzo[b]oxepin-8-yl)-2-oxo-oxazolidin-5-ylmethyl]-acetamide 7-11 (0.2 g), methylhydrazine (0.099 g), and triethylamine (0.22 g) were dissolved in ethanol (2 mL). The reaction was stirred at room temperature under an inert atmosphere for 20 h. The mixture was concentrated in vacuo. The residue was purified by flash silica gel chromatography using dichloromethane/methanol as gradient system. Yield: 40%. APCI (m/z): 357 (M+H).

### EXAMPLE 40

### (S)—N-[3-(2-Ethyl-4,5-dihydro-2H-6-oxa-1,2-diaza-benzo[e]azulen-8-yl)-2-oxo-oxazolidin-5-ylmethyl]-acetamide and (S)—N-[3-(1-Ethyl-4,5-dihydro-1H-6-oxa-1,2-diaza-benzo[e]azulen-8-yl)-2-oxo-oxazolidin-5-ylmethyl]-acetamide 40-1a, 40-1b

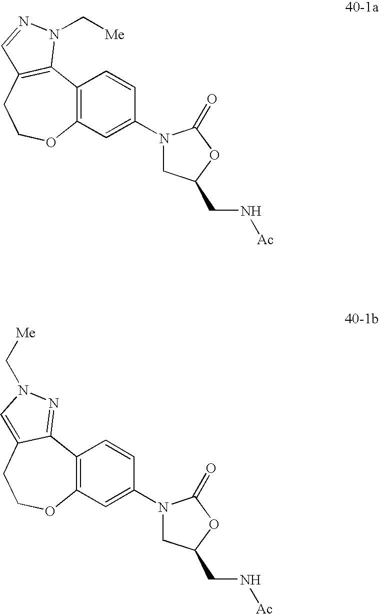

(S)—N-[3-(4-Dimethylaminomethylene-5-oxo-2,3,4,5-tetrahydro-benzo[b]oxepin-8-yl)-2-oxo-oxazolidin-5-ylmethyl]-acetamide 7-11 (0.2 g), ethylhydrazine oxalate (0.322 g), and triethylamine (0.22 g) were dissolved in ethanol (2 mL). The reaction was stirred at room temperature under an inert atmosphere for 20 h. The mixture was concentrated in vacuo. The residue was purified by flash silica gel chromatography using dichloromethane/methanol as the eluting gradient system. Yield: 35%. APCI (m/z): 371 (M+H).

### EXAMPLE 41

### (S)—N-[3-(2-Benzyl-4,5-dihydro-2H-6-oxa-1,2-diaza-benzo[e]azulen-8-yl)-2-oxo-oxazolidin-5-ylmethyl]-acetamide and (S)—N-[3-(1-Benzyl-4,5-dihydro-1H-6-oxa-1,2-diaza-benzo[e]azulen-8-yl)-2-oxo-oxazolidin-5-ylmethyl]-acetamide

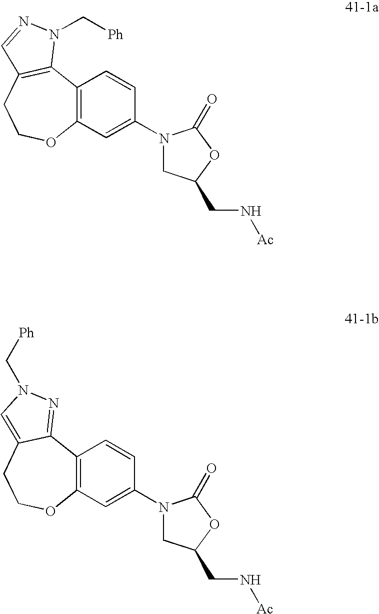

(S)—N-[3-(4-Dimethylaminomethylene-5-oxo-2,3,4,5-tetrahydro-benzo[b]oxepin-8-yl)-2-oxo-oxazolidin-5-ylmethyl]-acetamide (0.2 g), benzylhydrazine dihydrochloride (0.418 g), and triethylamine (0.22 g) were dissolved in ethanol (2 mL). The reaction was stirred at room temperature under an inert atmosphere for 20 h. The solvents were evaporated. The residue was purified by flash silica gel chromatography using dichloromethane/methanol as the eluting gradient system. Yield: 65%. APCI (m/z): 433 (M+H).

### EXAMPLE 42

### (S)—N-[2-Oxo-3-(2-phenethyl-4,5-dihydro-2H-6-oxa-1,2-diaza-benzo[e]azulen-8-yl)-oxazolidin-5-ylmethyl]-acetamide and (S)—N-[2-Oxo-3-(1-phenethyl-4,5-dihydro-1H-6-oxa-1,2-diaza-benzo[e]azulen-8-yl)-oxazolidin-5-ylmethyl]-acetamide

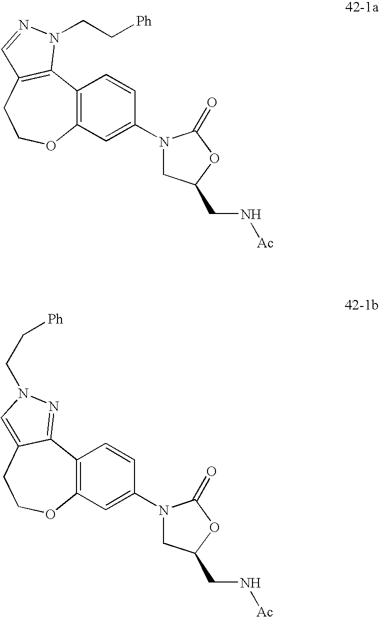

(S)—N-[3-(4-Dimethylaminomethylene-5-oxo-2,3,4,5-tetrahydro-benzo[b]oxepin-8-yl)-2-oxo-oxazolidin-5-ylmethyl]-acetamide (0.2 g), phenethylhydrazine sulfate (0.5 g), and triethylamine (0.22 g) were dissolved in ethanol (2 mL). The reaction mixture was stirred at room temperature under an inert atmosphere for 20 h. The solvent was evaporated. The residue was purified by flash silica gel chromatography using dichloromethane/methanol as the eluting gradient system. Yield: 53%. APCI (m/z): 445 (M+H).

### EXAMPLE 43

The following illustrates representative pharmaceutical dosage forms, containing a compound of Formula I (“Invention Compound”), for therapeutic or prophylactic use in humans.

| (i) Tablet | mg/tablet |
|---|---|
| ‘Invention Compound’ | 10-1000 |
| Lactose | 50.0 |
| Corn Starch (for mix) | 10.0 |
| Corn Starch (paste) | 10.0 |
| Magnesium Stearate (1%) | 3.0 |
| | 300.0 |

The invention compound, lactose, and corn starch (for mix) are blended to uniformity. The corn starch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of pathogenic bacterial infections.

| (ii) Tablet | mg/capsule |
|---|---|
| ‘Invention Compound | 10-1000 |
| Colloidal Silicon Dioxide | 1.5 |
| Lactose | 465.5 |
| Pregelatinized Starch | 120.0 |
| Magnesium Stearate (1%) | 3.0 |
| | 600.0 |

| (iii) Preparation for | | |
|---|---|---|
| Oral Solution | Amount | |
| ‘Invention Compound’ | 10-1000 | mg |
| Sorbitol Solution (70% N.F.) | 40 | mL |
| Sodium Benzoate | 20 | mg |
| Saccharin | 5 | mg |
| Cherry Flavor | 20 | mg |
| Distilled Water q.s. | 100 | mL |

The sorbitol solution is added to 40 mL of distilled water, and the invention compound is dissolved therein. The saccharin, sodium benzoate, flavor, and dye are added and dissolved. The volume is adjusted to 100 mL with distilled water. Each milliliter of syrup contains 4 mg of invention compound.

(iv) Parenteral Solution

In a solution of 700 mL of propylene glycol and 200 mL of water for injection is suspended 20 g of an invention compound. After suspension is complete, the pH is adjusted to 6.5 with 1 N hydrochloric acid, and the volume is made up to 1000 mL with water for injection. The Formulation is sterilized, filled into 5.0 mL ampoules each containing 2.0 mL, and sealed under nitrogen.

| | Amount |
|---|---|
| (v) Injection 1 (1 mg/mL) | |
| ‘Invention Compound’ | 1-1000 |
| Dibasic Sodium Phosphate | 12.0 |
| Monobasic Sodium Phosphate | 0.7 |
| Sodium Chloride | 4.5 |
| 1.0 N Sodium hydroxide solution | q.s. |
| (pH adjustment to 7.0-7.5) | |
| Water for injection | q.s. ad 1 mL |
| (vi) Injection 2 (10 mg/mL) | |
| ‘Invention Compound’ | 1-1000 |
| Dibasic Sodium Phosphate | 1.1 |
| Monobasic Sodium Phosphate | 0.3 |
| Polyethylene glyco 400 | 200.0 |
| 0.1 N hydrochloric acid solution | q.s. |
| (pH adjustment to 7.0-7.5) | |
| Water for injection | q.s. ad 1 mL |
| (vii) Injection 2 (10 mg/mL) | |
| ‘Invention Compound’ | 1-1000 |
| Oleic Acid | 10.0 |
| Trichloromonofluoromethane | 5,000.0 |
| Dichlorodifluoromethane | 10,000.0 |
| Dichlorotetrafluoroethane | 5,000.0. |

All patents, and patent documents are incorporated by reference herein, as though individually incorporated by reference. The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

1. A compound of formula I: 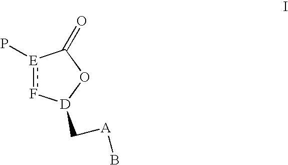
or a pharmaceutically acceptable salt thereof wherein:
A is O,
NH, or
S;
B is
C(═O)R₁,
C(═S)R₁,
heterocylco,
heteroaryl,
C(═O)-heterocyclo, or
C(═O)-heteteroaryl;
D is N, E is C, F is CH, and “—” is a bond, or D is CH, E is N, F is CH₂, and “—” is absent;
P is 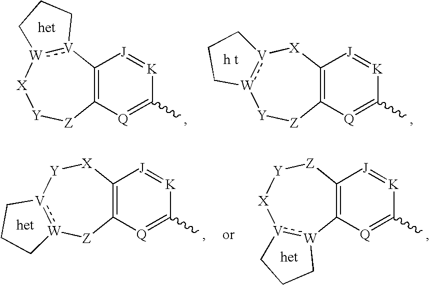
wherein “ [symbol] ” indicates the point of attachment; and 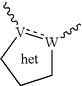
is 5-membered heterocyclo or heteroaryl, wherein “ [symbol] ” indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
V and W independently are CH or N when “—” is absent; or are C when “—” is a bond;
X, Y, Z independently are O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂,
provided that at least one of X, Y, or Z is NR₅,
N(C═O)R₅,
N(C═O)OR₅,
NSO₂R₅,
NSO₂NR₅,
O,
S,
SO, or
SO₂;
J, K, Q independently are CR₂ or N, with the proviso that when any one of J, K, or Q is N, then the other two are CR₂;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is as defined above.

2. The compound of claim 1, wherein 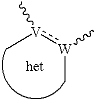
is

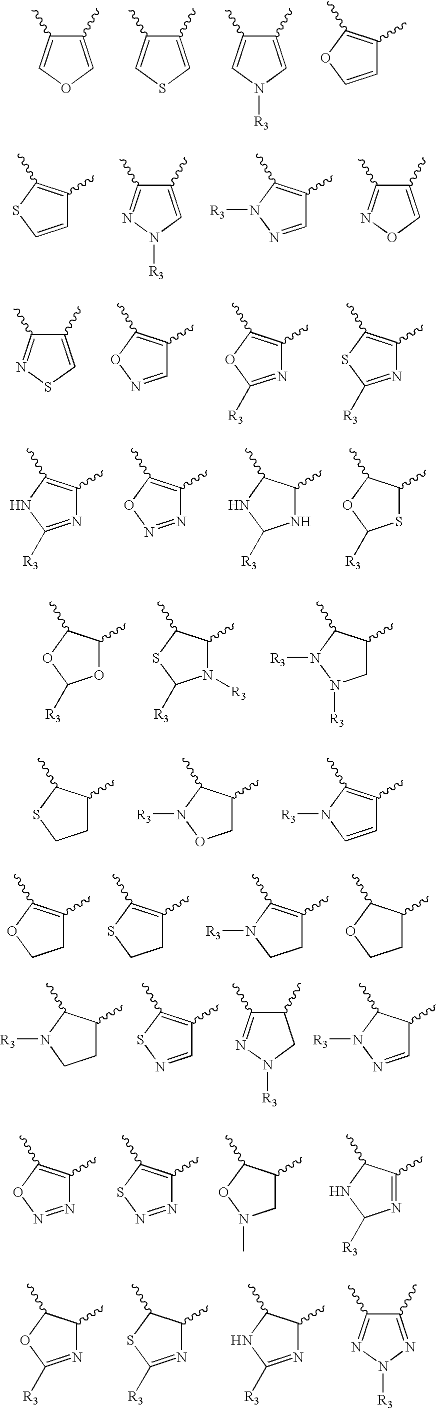

3. The compound of claim 1 as designated in formula IA.

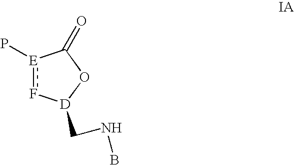

4. The compound of claim 1 as designated in formula IB.

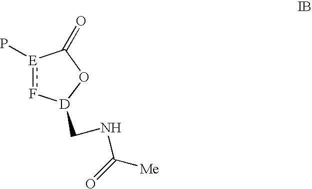

5. The compound of claim 1 as designated in formula IC.

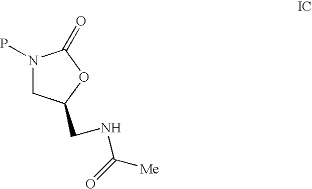

6. The compound of claim 5, wherein P is

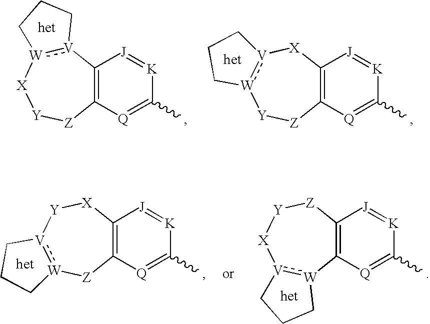

7. The compound of claim 6, wherein P is 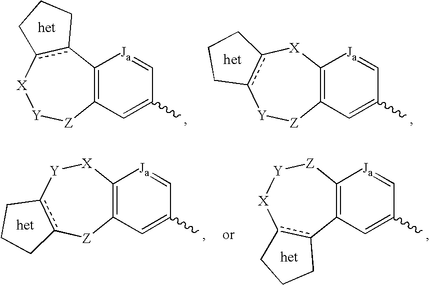
wherein Jₐ is N or CR₁₀, wherein R₁₀ is H or F. and wherein only one or two of X, Y, or Z is NR₅, N(C═O)R₅, N(C═O)OR₅, NSO₂R₅, NSO₂NR₅, O, S, SO, or SO₂.

8. A compound of formula II 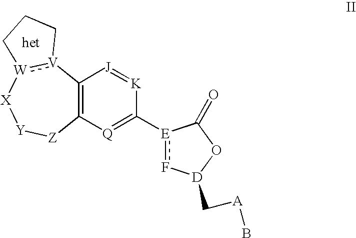
or a pharmaceutically acceptable salt thereof wherein:
A is O,
NH, or
S;
B is
C(═O)R₁,
C(═S)R₁,
heterocylco,
heteroaryl,
C(═O)-heterocyclo, or
C(═O)-heteteroaryl;
D is N, E is C, F is CH, and “—” is a bond, or D is CH, E is N, F is CH₂, and “—” is absent; 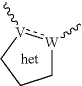
is 5-membered heterocyclo or heteroaryl, wherein “ [symbol] ” indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
V and W independently are CH or N when “—” is absent; or are C when “—” is a bond;
X, Y, Z independently are O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂,
provided that at least one of X, Y, or Z is NR₅,
N(C═O)R₅,
N(C═O)OR₅,
NSO₂R₅,
NSO₂NR₅,
O,
S,
SO, or
SO₂;
J, K, Q independently are CR₂ or N, with the proviso that when any one of J, K, or Q is N, then the other two are CR₂;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₅ is H,
(C₁-C₈)alkyl,
(C₃-C₆)cycloalkyl,
aryl,
(CH₂ₙ-aryl,
heterocyclo,
(CH₂)ₙ-heterocyclo,
heteroaryl, or
(CH₂)ₙ-heteroaryl;
wherein n is as defined above.

9. The compound of claim 9, wherein 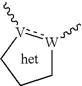
is as defined in claim 2.

10. The compound of claim 9 as designated in formula IIA.

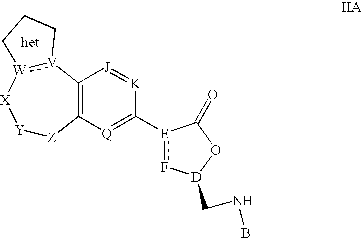

11. The compound of claim 9 as designated in formula IIB.

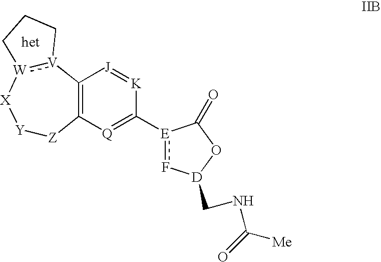

12. The compound of claim 9 as designated in formula IIC.

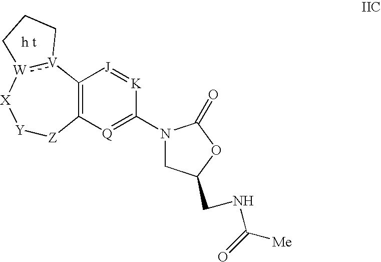

13. The compound of claim 9 as designated in formula IID 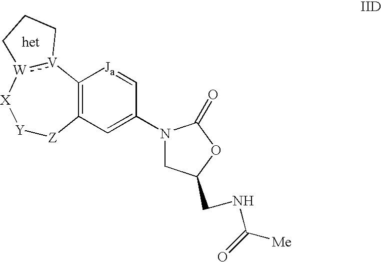
wherein Jₐ is N or CR₁₀, wherein R₁₀ is H or F. and wherein only one or two of X, Y, or Z is NR₅, N(C═O)R₅, N(C═O)OR₅, NSO₂R₅, NSO₂NR₅, O, S, SO, or SO₂.

14. The compound of claim 9 as designated in formula IIE, wherein only one or two of X, Y, or Z is NR₅, N(C═O)R₅, N(C═O)OR₅, NSO₂R₅, NSO₂NR₅, O, S, SO, or SO₂.

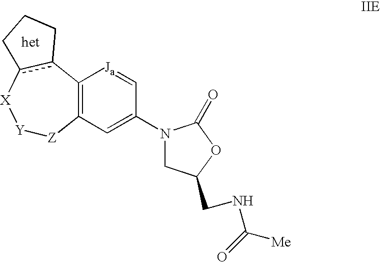

15. The compound of claim 9 as designated in formula IIF, wherein Xₐ is NR₅, N(C═O)R₅, N(C═O)OR₅, NSO₂R₅, NSO₂NR₅, O, S, SO, or SO₂.

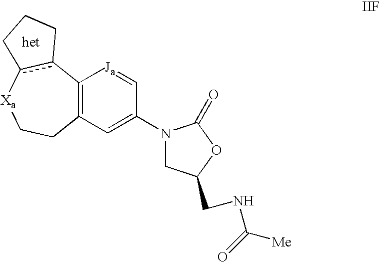

16. The compound of claim 9 as designated in formula IIG, wherein Yₐ is NR₅, N(C═O)R₅, N(C═O)OR₅, NSO₂R₅, NSO₂NR₅, O, S, SO, or SO₂.

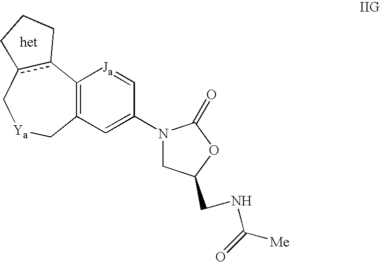

17. The compound of claim 9 as designated in formula IIH, wherein Zₐ is NR₅, N(C═O)R₅, N(C═O)OR₅, NSO₂R₅, NSO₂NR₅, O, S, SO, or SO₂.

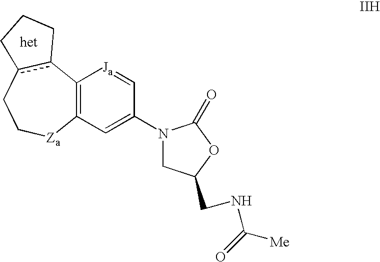

18. A compound of formula III 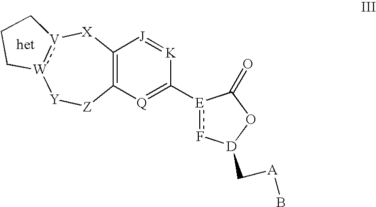
or a pharmaceutically acceptable salt thereof wherein:
A is O,
NH, or
S;
B is
C(═O)R₁,
C(═S)R_{,}
heterocylco,
heteroaryl,
C(═O)-heterocyclo, or
C(═O)-heteteroaryl;
D is N, E is C, F is CH, and “—” is a bond, or D is CH, E is N, F is CH₂, and “—” is absent; 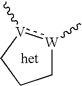
is 5-membered heterocyclo or heteroaryl, wherein “ [symbol] ” indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
V and W independently are CH or N when “—” is absent; or are C when “—” is a bond;
X, Y, Z independently are O=C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂,
provided that at least one of X, Y, or Z is NR₅,
N(C═O)R₅,
N(C═O)OR₅,
NSO₂R₅,
NSO₂NR₅,
O,
S,
SO, or
SO₂;
J, K, Q independently are CR₂ or N, with the proviso that when any one of J, K, or Q is N, then the other two are CR₂;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is as defined above.

19. The compound of claim 18, wherein 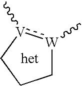
is as defined in claim 2.

20. The compound of claim 18 as designated in formula IIIA.

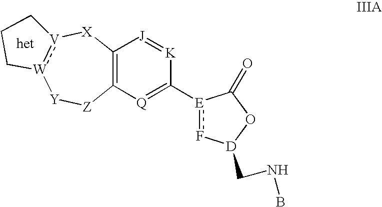

21. The compound of claim 19 as designated in formula IIIB.

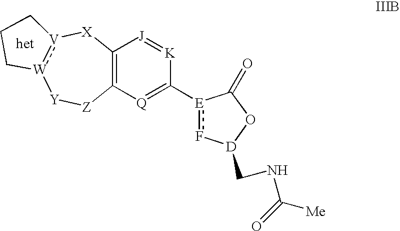

22. The compound of claim 19 as designated in formula IIIC.

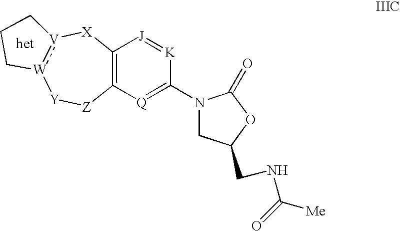

23. The compound of claim 19 as designated in formula IIID 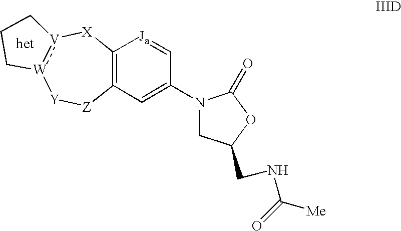
wherein Jₐ is N or CR₁₀, wherein R₁₀ is H or F. and wherein only one or two of X, Y, or Z is NR₅, N(C═O)R₅, N(C═O)OR₅, NSO₂R₅, NSO₂NR₅, O, S, SO, or SO₂.

24. The compound of claim 19 as designated in formula IIE, wherein only one or two of X, Y, or Z is NR₅, N(C═O)R₅, N(C═O)OR₅, NSO₂R₅, NSO₂NR₅, O, S, SO, or SO₂.

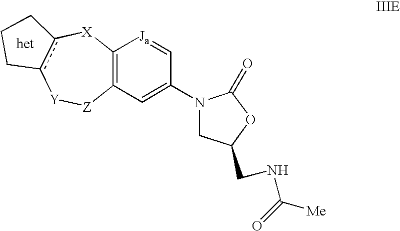

25. The compound of claim 19 as designated in formula IIIF, wherein Xₐ is NR₅, N(C═O)R₅, N(C═O)OR₅, NSO₂R₅, NSO₂NR₅, O, S, SO, or SO₂.

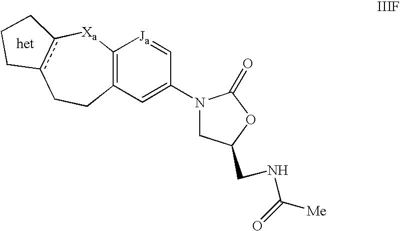

26. The compound of claim 19 as designated in formula IIIG, wherein Yₐ is NR₅, N(C═O)R₅, N(C═O)OR₅, NSO₂R₅, NSO₂NR₅, O, S, SO, or SO₂.

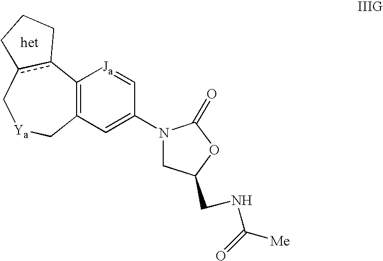

27. The compound of claim 19 as designated in formula IIIH, wherein Zₐ is NR₅, N(C═O)R₅, N(C═O)OR₅, NSO₂R₅, NSO₂NR₅, O, S, SO, or SO₂.

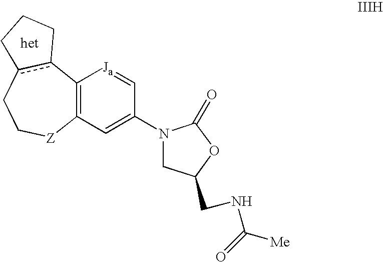

28. A compound of formula IV: 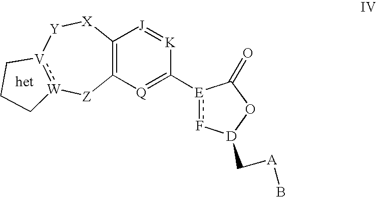
or a pharmaceutically acceptable salt thereof wherein:
A is O,
NH, or
S;
B is
C(═O)R₁,
C(═S)R₁,
heterocylco,
heteroaryl,
C(═O)-heterocyclo, or
C(═O)-heteteroaryl;
D is N, E is C, F is CH, and “—” is a bond, or D is CH, E is N, F is CH₂, and “—” is absent; 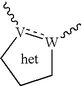
is 5-membered heterocyclo or heteroaryl, wherein “ [symbol] ” indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
V and W independently are CH or N when “—” is absent; or are C when “—” is a bond;
X, Y, Z independently are O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂,
provided that at least one of X, Y, or Z is NR₅,
N(C═O)R₅,
N(C═O)OR₅,
NSO₂R₅,
NSO₂NR₅,
O,
S,
SO, or
SO₂;
J, K, Q independently are CR₂ or N, with the proviso that when any one of J, K, or Q is N, then the other two are CR₂;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2,or3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is as defined above.

29. The compound of claim 28, wherein 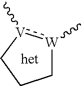
is as defined in claim 2.

30. The compound of claim 28 as designated in formula IVA.

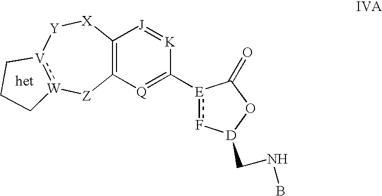

31. The compound of claim 28 as designated in formula IVB.

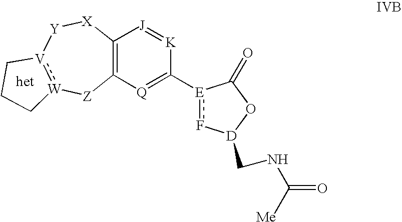

32. The compound of claim 28 as designated in formula IVC.

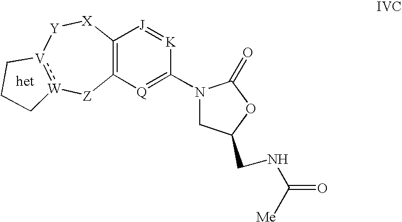

33. The compound of claim 28 as designated in formula IVD 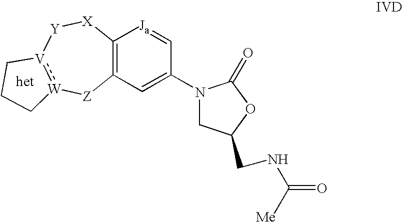
wherein Jₐ is N or CR₁₀, wherein R₁₀ is H or F. and wherein only one or two of X, Y, or Z is NR₅, N(C═O)R₅, N(C═O)OR₅, NSO₂R₅, NSO₂NR₅, O, S, SO, or SO₂.

34. The compound of claim 28 as designated in formula IVE, wherein only one or two of X, Y, or Z is NR₅, N(C═O)R₅, N(C═O)OR₅, NSO₂R₅, NSO₂NR₅, O, S, SO, or SO₂. 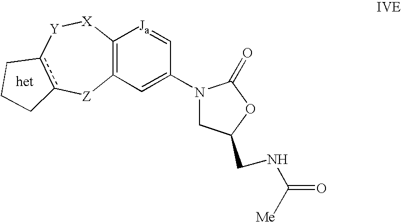
wherein R₈ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl, wherein n is 0, 1, 2, or 3; or taken together R₈ and R₉ are bonded to the same C and form C═O.

35. The compound of claim 28 as designated in formula IVF, wherein Xₐ is NR₅, N(C═O)R₅, N(C═O)OR₅, NSO₂R₅, NSO₂NR₅, O, S, SO, or SO₂.

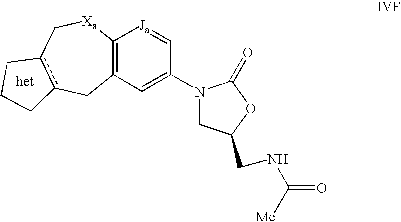

36. The compound of claim 28 as designated in formula IVG, wherein Yₐ is NR₅, N(C═O)R₅, N(C═O)OR₅, NSO₂R₅, NSO₂NR₅, O, S, SO, or SO₂.

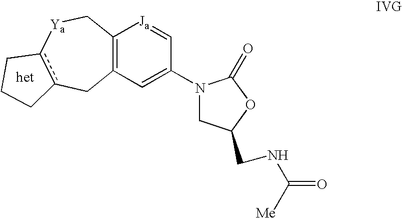

37. The compound of claim 28 as designated in formula IVH, wherein Zₐ is NR₅, N(C═O)R₅, N(C═O)OR₅, NSO₂R₅, NSO₂NR₅, O, S, SO, or SO₂.

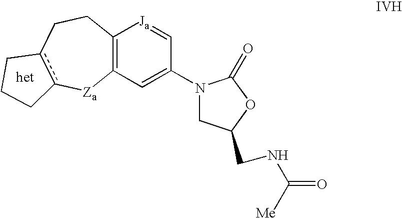

38. A compound of formula V: 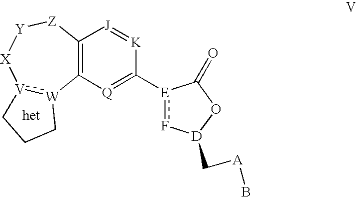
or a pharmaceutically acceptable salt thereof wherein:
A is O,
NH, or
S;
B is
C(═O)R₁,
C(═S)R₁,
heterocylco,
heteroaryl,
C(═O)-heterocyclo, or
C(═O)-heteteroaryl;
D is N, E is C, F is CH, and “—” is a bond, or D is CH, E is N, F is CH₂, and “—” is absent; 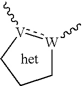
is 5-membered heterocyclo or heteroaryl, wherein “ [symbol] ” indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R, OR₅, OC(═O)R₁, NR₆R₇, NHR₅, N(C═O)R₅, NH(C═O)OR₅, NHSO₂R₅, NHSO₂NR₅;
V and W independently are CH or N when “—” is absent; or are C when “—” is a bond;
X, Y, Z independently are O═C,
CH₂,
CHR₃,
CHR₄,
CR₃R₄,
NR₅,
N(C═O)R₅,
N(C═O)OR₅,
NSO₂R₅,
NSO₂NR₅,
O,
S,
SO, or
SO₂,
provided that at least one of X, Y, or Z is NR₅,
N(C═O)R₅,
N(C═O)OR₅,
NSO₂R₅,
NSO₂NR₅,
O,
S,
SO, or
SO₂;
J, K, Q independently are CR₂ or N, with the proviso that when any one of J, K, or Q is N, then the other two are CR₂;
R₁ is H,
(C₁-C₈)alkyl,
(C₃-C₆)cycloalkyl,
O—C₁-C₄)alkyl,
O—C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is as defined above.

39. The compound of claim 38, wherein 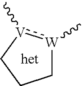
is as defined in claim 2.

40. The compound of claim 38 as designated in formula VA.

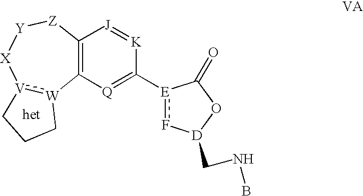

41. The compound of claim 38 as designated in formula VB.

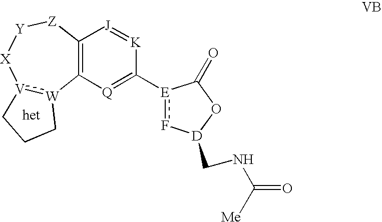

42. The compound of claim 38 as designated in formula VC.

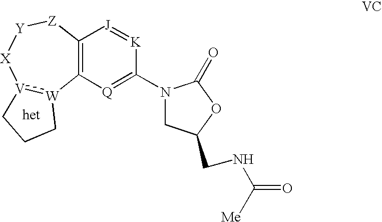

43. The compound of claim 38 as designated in formula VD 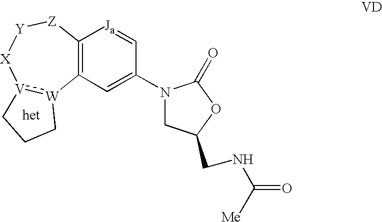
wherein Jₐ is N or CR₁₀, wherein R₁₀ is H or F. and wherein only one or two of X, Y, or Z is NR₅, N(C═O)R₅, N(C═O)OR₅, NSO₂R₅, NSO₂NR₅, O, S, SO, or SO₂.

44. The compound of claim 38 as designated in formula IIE, wherein only one or two of X, Y, or Z is NR₅, N(C═O)R₅, N(C═O)OR₅, NSO₂R₅, NSO₂NR₅, O, S, SO, or SO₂.

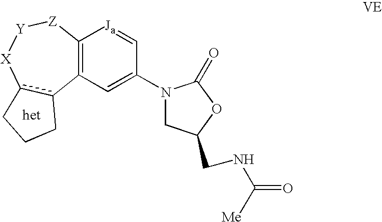

45. The compound of claim 38 as designated in formula VF, wherein Xₐ is NR₅, N(C═O)R₅, N(C═O)OR₅, NSO₂R₅, NSO₂NR₅, O, S, SO, or SO₂.

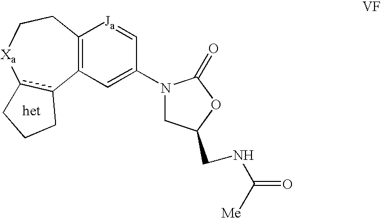

46. The compound of claim 38 as designated in formula VG, wherein Yₐ is NR₅, N(C═O)R₅, N(C═O)OR₅, NSO₂R₅, NSO₂NR₅, O, S, SO, or SO₂.

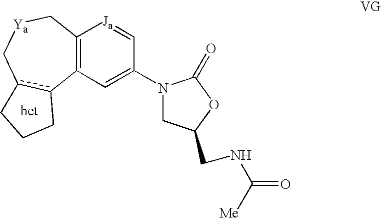

47. The compound of claim 38 as designated in formula VH, wherein Zₐ is NR₅, N(C═O)R₅, N(C═O)OR₅, NSO₂R₅, NSO₂NR₅, O, S, SO, or SO₂.

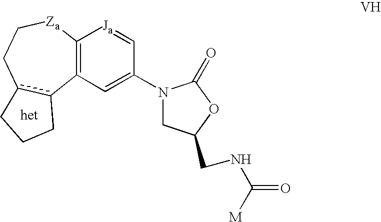

49. A compound which is:
(S)—N-[3-(4,5-Dihydro-2H-6-oxa-1,2-diaza-benzo[e]azulen-8-yl)-2-oxo-oxazolidin-5-ylmethyl]-acetamide;
(S)—N-[3-(2-Methyl-4,5-dihydro-2H-6-oxa-1,2-diaza-benzo[e]azulen-8-yl)-2-oxo-oxazolidin-5-ylmethyl]-acetamide;
(S)—N-[3-(1-Methyl-4,5-dihydro-1H-6-oxa-1,2-diaza-benzo[e]azulen-8-yl)-2-oxo-oxazolidin-5-ylmethyl]-acetamide;
(S)—N-[3-(2-Ethyl-4,5-dihydro-2H-6-oxa-1,2-diaza-benzo[e]azulen-8-yl)-2-oxo-oxazolidin-5-ylmethyl]-acetamide;
(S)—N-[3-(1-Ethyl-4,5-dihydro-1H-6-oxa-1,2-diaza-benzo[e]azulen-8-yl)-2-oxo-oxazolidin-5-ylmethyl]-acetamide;
(S)—N-[3-(2-Benzyl-4,5-dihydro-2H-6-oxa-1,2-diaza-benzo[e]azulen-8-yl)-2-oxo-oxazolidin-5-ylmethyl)-acetamide;
(S)—N-[3-(1-Benzyl-4,5-dihydro-1H-6-oxa-1,2-diaza-benzo[e]azulen-8-yl)-2-oxo-oxazolidin-5-ylmethyl]-acetamide;
(S)—N-[2-Oxo-3-(2-phenethyl-4,5-dihydro-2H-6-oxa-1,2-diaza-benzo[e]azulen-8-yl)-oxazolidin-5-ylmethyl]-acetamide;
(S)—N-[2-Oxo-3-(1)-phenethyl4,5-dihydro-1H-6-oxa-1,2-diaza-benzo[e]azulen-8-yl)-oxazolidin-5-ylmethyl]-acetamide;
(S)—N-[2-Oxo-3-(3-phenyl-4,5-dihydro-2H-6-oxa-1,2-diaza-benzo[e]azulen-8-yl)-oxazolidin-5-ylmethyl]-acetamide;
(S)—N-[3-(2,6-Dihydro4H-5-oxa-1,2-diaza-benzo[e]azulen-8-yl)-2-oxo-oxazolidin-5-ylmethyl]-acetamide;
(S)—N-[3-(5,6-Dihydro-2H4-oxa-1,2-diaza-benzo[e]azulen-8-yl)-2-oxo-oxazolidin-5-ylmethyl]-acetamide;
(S)—N-[2-Oxo-3-(2,4,5,6-tetrahydro-1,2,6-triaza-benzo[e]azulen-8-yl)-oxazolidin-5-ylmethyl]-acetamide;
(S)—N-[2-Oxo-3-(2,4,5,6-tetrahydro 1,2,5-triaza-benzo[e]azulen-8-yl)-oxazolidin-5-ylmethyl]-acetamide;
(S)—N-[2-Oxo-3-(2,4,5,6-tetrahydro-1,2,4-triaza-benzo[e]azulen-8-yl)-oxazolidin-5-ylmethyl]-acetamide;
(S)—N-[3-(4,5-Dihydro-2H-6-thia-1,2-diaza-benzo[e]azulen-8-yl)-2-oxo-oxazolidin-5-ylmethyl]-acetamide;
(S)—N-[3-(2,6-Dihydro-4H-5-thia-1,2-diaza-benzo[e]azulen-8-yl)-2-oxo-oxazolidin-5-ylmethyl]-acetamide;
(S)—N-[3-(5,6-Dihydro-2H-4-thia-1,2-diaza-benzo[e]azulen-8-yl)-2-oxo-oxazolidin-5-ylmethyl]-acetamide;
(S)—N-[3-(6,6-Dioxo-2,4,5,6-tetrahydro-616-thia-1,2-diaza-benzo[e]azulen-8-yl)-2-oxo-oxazolidin-5-ylmethyl]-acetamide;
(S)—N-[3-(5,5-Dioxo-2,4,5,6-tetrahydro-516-thia-1,2-diaza-benzo[e]azulen-8-yl)-2-oxo-oxazolidin-5-ylmethyl]-acetamide;
(S)—N-[3-(4,4-Dioxo-2,4,5,6-tetrahydro-416-thia-1,2-diaza-benzo[e]azulen-8-yl)-2-oxo-oxazolidin-5-ylmethyl]-acetamide;
(S)—N-[3-(4,5-Dihydro-1,6-dioxa-2-aza-benzo[e]azulen-8-yl)-2-oxo-oxazolidin-5-ylmethyl]-acetamide;
(S)—N-[3-(4H,6H-1,5-Dioxa-2-aza-benzo[e]azulen-8-yl)-2-oxo-oxazolidin-5-ylmethyl]-acetamide;
(S)—N-[3-(5,6-Dihydro-1,4-dioxa-2-aza-benzo[e]azulen-8-yl)-2-oxo-oxazolidin-5-ylmethyl]-acetamide;
(S)—N-[3-(5,6-Dihydro4H-1-oxa-2,6-diaza-benzo[e]azulen-8-yl)-2-oxo-oxazolidin-5-ylmethyl]-acetamide;
(S)—N-[3-(5,6-Dihydro-4H-1-oxa-2,5-diaza-benzo[e]azulen-8-yl)-2-oxo-oxazolidin-5-ylmethyl]-acetamide;
(S)—N-[3-(5,6-Dihydro-4H-1-oxa-2,4-diaza-benzo[e]azulen-8-yl)-2-oxo-oxazolidin-5-ylmethyl]-acetamide;
(S)—N-[3-(4,5-Dihydro-1-oxa-6-thia-2-aza-benzo[e]azulen-8-yl)-2-oxo-oxazolidin-5-ylmethyl]-acetamide;
(S)—N-[3-(4H,6H-1-Oxa-5-thia-2-aza-benzo[e]azulen-8-yl)-2-oxo-oxazolidin-5-ylmethyl]-acetamide;
(S)—N-[3-(5,6-Dihydro-1-oxa4thia-2-aza-benzo[e]azulen-8-yl)-2-oxo-oxazolidin-5-ylmethyl]-acetamide;
(S)—N-[3-(4,5-Dihydro-1-oxa-6-thia-2-aza-benzo[e]azulen-8-yl)-2-oxo-oxazolidin-5-ylmethyl]-acetamide;
(S)—N-[3-(5,5-Dioxo-5,6-dihydro4H-1-oxa-516-thia-2-aza-benzo[e]azulen-8-yl)-2-oxo-oxazolidin-5-ylmethyl]-acetamide; or
(S)—N-[3-(5,6-Dihydro-1-oxa4-thia-2-aza-benzo[e]azulen-8-yl)-2-oxo-oxazolidin-5-ylmethyl]-acetamide.

50. A pharmaceutical formulation comprising a compound of claim 1 admixed with a pharmaceutically acceptable diluent, carrier, or excipient.

51. A method of treating a bacterial infection in a mammal, comprising administering to a mammal in need thereof an effective amount of a compound of claim 1.
